(19) United States
(12) Patent Application Publication
(10) Pub. No.: US 2023/0172899 A1
(43) Pub. Date: Jun. 8, 2023

(54) CANNABINOID COMPOSITIONS

(71) Applicant: The Queen's Medical Center, Honolulu, HI (US)

(72) Inventors: Reinhold PENNER, Honolulu, HI (US); Ram P. NEUPANE, Aiea, HI (US)

(21) Appl. No.: 17/906,735

(22) PCT Filed: Mar. 19, 2021
(86) PCT No.: PCT/US2021/023291

§ 371 Date: Sep. 19, 2022

Related U.S. Application Data
(60) Provisional application No. 62/992,582, filed on Mar. 20, 2020.

(51) Int. Cl. A61K 31/352; A61K 31/05; A61K 36/185; A61K 31/192

(52) U.S. Cl. CPC A61K 31/352; A61K 31/05; A61K 36/185; A61K 31/192; A61K 2236/37

(57) ABSTRACT

Described herein are pharmaceutical compositions comprising cannabinoids. In some embodiments, such compositions are useful for the treatment of inflammatory, autoimmune diseases or disorders, cancer, or neurodegenerative diseases. Further provided herein are pharmaceutical compositions comprising combinations of cannabinoids possessing entourage effects.

### CROSS-REFERENCE

This patent application claims the benefit of U.S. Provisional Patent Application No. 62/992,582, filed on Mar. 20, 2020 which is incorporated herein by reference in its entirety.

### BACKGROUND

Chronic diseases affect millions of individuals across the globe. There exists a need for new medicines for the treatment of chronic diseases and conditions such as inflammation, cancer, neurodegeneration, autoimmune, and pain. A potential source of medicines for the prevention and treatment of these diseases are extracts and compositions comprising cannabinoids.

### SUMMARY OF THE INVENTION

In some embodiments, the invention provides compositions, methods and systems for the treatment or amelioration of a disease state in the patient comprising administering to the patient a combination of at least two cannabinoids. In some embodiments, provided herein are pharmaceutical compositions for the treatment or amelioration of a disease in a patient in need thereof, the pharmaceutical composition comprising: at least one acidic cannabinoid compound, at least one non-acidic cannabinoid compound and a pharmaceutical excipient. In some embodiments, the pharmaceutical compositions comprising an acidic cannabinoid compound and a non-acidic cannabinoid compound are extracts from a cannabis plant. In some embodiments, the non-acidic cannabinoid compound and acidic cannabinoid compound are in extracts from a cannabis plant. In some embodiments, the non-acidic cannabinoid compound are in extracts from a cannabis plant. In some embodiments, the acidic cannabinoid compound are in extracts from a cannabis plant. In some embodiments, the non-acidic cannabinoid compound are derived from extracts from a cannabis plant. In some embodiments, the acidic cannabinoid compound are derived from extracts from a cannabis plant. In some embodiments, the acidic cannabinoid compound and non-acidic cannabinoid compound are synthesized. In some embodiments, the acidic cannabinoid compound is extracted from a cannabis plant. In some embodiments, the extraction process is chosen from the group consisting of supercritical fluid extraction, solvent extraction, ultrasound-assisted extraction, or combinations thereof. In some embodiments, the solvent is an alcohol, hexane or combinations thereof. In some embodiments, the alcohol is ethanol or isopropanol. In yet other instances, the extraction process does not decarboxylate the acidic cannabinoid compounds in the plant extract. In still other embodiments, the extraction process yields an acidic cannabinoid compound of about 90% purity, about 95% purity or about 99% purity. In yet other instances, the non-acidic-cannabinoid compound is extracted from a cannabis plant. In still other instances, the extraction process is chosen from the group consisting of supercritical fluid extraction, solvent extraction, ultrasound-assisted extraction, microwave-assisted extracted or combinations thereof. In still other embodiments, the cannabis plant extract is processed to decarboxylate acidic-cannabinoid compounds in the cannabis plant extract. In other instances, the decarboxylation process includes a heating step. In still other instances, the heating step is maintained at above 100° C. for a predetermined time period. In yet other instances, the heating step is maintained at above 100° C. for a predetermined time period. In some embodiments, the extraction and decarboxylation process yields a non-acidic cannabinoid compound of about 90% purity, about 95% purity or about 99% purity. In some embodiments, the composition reduces an influx of extracellular Ca²⁺ in a cell as compared to the individual acidic cannabinoid compound and non-acidic cannabinoid compound. In yet other instances, the acidic cannabinoid compound is cannabidiolic acid (CBDA), cannabigerolic acid (CBGA), tetrahydrocannabinolic Acid (THCA), tetrahydrocannabivarinic acid (CBDVA), cannabigerovarinic acid (CBGVA), cannabicyclolic acid (CBLA), cannabinolic acid (CBNA), cannabichromenic acid (CBCA), tetrahydrocannabivarinic acid (THCVA), or combinations thereof. In still other instances, the composition comprises at least two acidic cannabinoid compounds. In some instances, the acidic cannabinoid compound is added in a purified form. In other instances, the non-acidic cannabinoid compound is CBD, CBG, THC, CBDV, CBGV, CBL, CBN, CBC, THCV, or combinations thereof. In still other instances, the composition comprises at least two non-acidic cannabinoid compounds. In yet other instances, the non-acidic cannabinoid compound is added in a purified form. In some instances, at least one acidic cannabinoid compound and at least one non-acidic cannabinoid compound is present at a molar ratio of between 1:1 and 1:200. In other instances, at least one non-acidic cannabinoid compound and at least one acidic cannabinoid compound is present at a molar ratio of between 1:1 and 1:200. In some embodiments, the composition treats or ameliorates an autoimmune disease, a neurodegenerative disease, an inflammatory condition, pain, cancer or combinations thereof. In yet other instances, at least one acidic cannabinoid compound is present in an extract of a cannabis plant, and at least one non-acidic cannabinoid compound is present in an extract of a cannabis plant. In other instances, the extract comprising at least one acidic cannabinoid compound and the extract comprising at least one non-acidic cannabinoid compound is combined at a ratio of between 1:1 and 1:200. In still other instances, the extract comprising at least one non-acidic cannabinoid compound and the extract comprising at least one acidic cannabinoid compound is combined at a ratio of between 1:1 and 1:200. In some instances, the acidic cannabinoid is CBGA. In yet other instances, the non-acidic cannabinoid is CBG, CBD, CBDV, THC or combinations thereof. In still other instances, the pharmaceutical excipient is chosen from the group consisting of buffer, diluent, disintegrant, glidant, lubricant, coating, carrier, controlled release agent, adjuvant, vehicle, binder, emulsifying agent, or combinations thereof.

Also provided herein are methods, systems and compositions for the treatment or amelioration of an autoimmune disease comprising: at least one acidic cannabinoid compound, at least one non-acidic cannabinoid compound and a pharmaceutical excipient. Also provided herein are compositions, methods and systems for treating or ameliorating cancer, the method comprising administering to a patient in need thereof a therapeutically effective amount of a pharmaceutical composition comprising at least one acidic cannabinoid compound, at least one non-acidic cannabinoid compound and a pharmaceutical excipient. In some instances, the acidic cannabinoid compound is CBDA, CBGA, THCA, CBDVA, CBGVA, CBLA, CBNA, CBCA, THCVA, or combinations thereof. In yet other instances, the composition comprises at least two acidic cannabinoid compounds. In still other instances, the acidic cannabinoid compound is added in a purified form. In some embodiments, the non-acidic cannabinoid compound is CBD, CBG, THC, CBL, CBCV or combinations thereof. In still other embodiments, the composition comprises at least two non-acidic cannabinoid compounds. In yet other embodiments, the non-acidic cannabinoid compound is added in a purified form. In still other instances, at least one acidic cannabinoid compound and at least one non-acidic cannabinoid compound is present at a molar ratio of between 1:1 and 1:200. In yet other instances, at least one non-acidic cannabinoid compound and at least one acidic cannabinoid compound is present at a molar ratio of between 1:1 and 1:200.

Provided herein are compositions, methods and systems for treating or ameliorating cancer, the method comprising administering to a patient in need thereof a therapeutically effective amount of a pharmaceutical composition comprising at least one acidic cannabinoid compound, at least one non-acidic cannabinoid compound and a pharmaceutical excipient. Also provided herein are compositions, methods and systems for the treatment or amelioration of an inflammatory condition comprising: at least one acidic cannabinoid compound, at least one non-acidic cannabinoid compound and a pharmaceutical excipient. In addition, provided herein are compositions, methods and systems for the treatment or amelioration of a neurodegenerative condition comprising: at least one acidic cannabinoid compound, at least one non-acidic cannabinoid compound and a pharmaceutical excipient. Provided herein are compositions, methods, and systems for the treatment or amelioration of pain comprising: at least one acidic cannabinoid compound, at least one non-acidic cannabinoid compound and a pharmaceutical excipient. In some instances, the acidic cannabinoid compound is CBDA, CBGA, THCA, CBDVA, CBGVA, CBLA, CBNA, CBCA, THCVA, or combinations thereof. In yet other instances, the composition comprises at least two acidic cannabinoid compounds. In still other instances, the acidic cannabinoid compound is added in a purified form. In some embodiments, the non-acidic cannabinoid compound is CBD, CBG, THC, CBL, CBCV or combinations thereof. In still other embodiments, the composition comprises at least two non-acidic cannabinoid compounds. In yet other embodiments, the non-acidic cannabinoid compound is added in a purified form. In still other instances, at least one acidic cannabinoid compound and at least one non-acidic cannabinoid compound is present at a molar ratio of between 1:1 and 1:200. In yet other instances, at least one non-acidic cannabinoid compound and at least one acidic cannabinoid compound is present at a molar ratio of between 1:1 and 1:200.

Also provided herein are methods and systems for producing a composition comprising at least one acidic cannabinoid compound and at least one non-acidic cannabinoid compounds, the methods and systems comprising:
a. extracting a first feedstock from a cannabis plant to provide a first feedstock comprising an acidic cannabinoid;
b. extracting a second feedstock from the cannabis plant and decarboxylating the extracted second feedstock to provide a second feedstock comprising a non-acidic cannabinoid;
c. combining at least a fraction of the first feedstock comprising an acidic cannabinoid with at least a fraction of the second feedstock comprising a non-acidic cannabinoid thereby producing a composition comprising at least one acidic cannabinoid and at least one non-acidic cannabinoid.
   In some instances, the acidic cannabinoid is CBGA. In yet other instances, the non-acidic cannabinoid is CBG, CBD, CBDV or combinations thereof. In still other instances, the first feedstock is extracted using supercritical fluid, solvent, ultrasound-assisted, or combinations thereof. In some instances, the first feedstock is extracted using supercritical carbon dioxide. In other instances, the second feedstock is extracted using supercritical fluid, solvent, ultrasound-assisted, microwave-assisted or combinations thereof, and heated to at least 100° C. for a predetermined time period. In yet other instances, the composition reduces an influx of extracellular Ca²⁺ in a cell as compared to the individual acidic cannabinoid and non-acidic cannabinoid extracts. In still other instances, the composition modulates an inflammatory response in the cell by modulating the uptake of cytosolic Ca²⁺. In some instances, the composition modulates an inflammatory response in the cell by modulating the extrusion of cytosolic Ca²⁺. In other instances, the composition modulates an inflammatory response in an immune cell. In yet other instances, the acidic cannabinoid comprises cannabigerolic acid (CBGA) and the non-acidic cannabinoid comprises cannabidiol (CBD), wherein the molar ratio of CBGA to CBD is from about 1:1 to about 1:200. In still other instances, the non-acidic cannabinoid comprises cannabidiol acid (CBD) and the acidic cannabinoid comprises cannabigerolic acid (CBGA), wherein the molar ratio of CBD to CBGA is from about 1:1 to about 1:200. In yet other instances, the acidic cannabinoid comprises cannabigerolic acid (CBGA) and the non-acidic cannabinoid comprises cannabigerol (CBG), wherein the molar ratio of CBGA to CBG is from about 1:1 to about 1:200. In still other instances, the non-acidic cannabinoid comprises cannabidivarin (CBDV) and the acidic cannabinoid comprises cannabigerolic acid (CBGA), wherein the molar ratio of CBDV to CBGA is from about 1:1 to about 1:200. In some instances, the acidic cannabinoid comprises cannabigerolic acid (CBGA) and the non-acidic cannabinoid comprises cannabidivarin (CBDV), wherein the molar ratio of CBGA to CBDV is from about 1:1 to about 1:200. In other instances, the composition is formulated for administration to a subject. In still other instances, the composition is packaged into a container selected from the group consisting of a tube, ajar, a vial, a bag, a tray, a drum, a bottle, a syringe, and a can. In some instances, the container contains information describing directions for use in a subject. In still other instances, the subject is a human.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a Store-Operated Calcium Entry (SOCE) Fura-2 bioassay in various cell types, in accordance with embodiments. Traces in black indicate full activation by appropriate agonist (Tg: Thapsigargin 1 μM) as indicated in panels. Traces labeled Gd³⁺ represent data obtained in the presence of inhibitor compound (Gd³⁺: Gadolinium Chloride 1 μM).

FIG. 2 illustrates Fura-2 bioassays of over-expressed TRP ion channels involved in pain, in accordance with embodiments. Traces in black indicate full activation by appropriate agonists as indicated in panels. Traces in gray represent data obtained in the presence of inhibitor compound. Arrows indicate time of agonist application. PS: pregnenolone sulfate; AITC: allyl isothiocyanate. FIG. 2A depicts ion channel TRPM3. FIG. 2B depicts ion channel TRPM8. FIG. 2C depicts ion channel TRPA1. FIG. 2D depicts ion channel TRPV1.

FIG. 3 illustrates agonist-induced Ca²⁺ oscillations in three intact Jurkat T lymphocytes, in accordance with embodiments.

FIGS. 4A-4F illustrate whole-cell patch clamp electrophysiology of various ion channels in tetracycline-induced overexpressing HEK293 cells, in accordance with embodiments.

FIG. 4A shows activation of TRPV1, in accordance with embodiments. Left panel is averaged current development before, during and after agonist application (n=3-5, S.E.M.). Right panel is representative current-voltage traces extracted at the time of maximal current activation.

FIG. 4B shows activation of TRPM3, in accordance with embodiments. Left panel is averaged current development before, during and after agonist application (n=3-5, S.E.M.). Right panel is representative current-voltage traces extracted at the time of maximal current activation.

FIG. 4C shows activation of TRPA1, in accordance with embodiments. Left panel is averaged current development before, during and after agonist application (n=3-5, S.E.M.). Right panel is representative current-voltage traces extracted at the time of maximal current activation.

FIG. 4D shows activation of Kv1.3, in accordance with embodiments. Left panel shows averaged current development by voltage activation (Kv1.3). Right panel is representative current-voltage traces extracted at the time of maximal current activation. Right panel is representative current-voltage traces extracted at the time of maximal current activation.

FIG. 4E shows activation of I_{CRAC}, in accordance with embodiments. Left panel shows averaged current development by internal perfusion with 50 μM inositol 1,4,5-trisphosphate (IP₃). Right panel is representative current-voltage traces extracted at the time of maximal current activation.

FIG. 4F shows activation of TRPM8, in accordance with embodiments. Left panel shows an example cell activated with menthol. Right panel is representative current-voltage traces extracted at the time of maximal current activation.

FIG. 5 illustrates cytokine release in human immune cells, in accordance with embodiments.

FIG. 6 illustrates HPLC-UV (210 nm) traces of the terpene-deficient (TerpDefExt) and terpene-rich (TerpRichExt) extracts of the Cannabis plant material (NIDA Chemovar S04) and mixtures of commercial standards of terpenes and cannabinoids. TerpMixA (terpene standards): linalool (2), β-myrcene (13), terpinolene (14), limonene (18), α-pinene (22). TerpMixB (terpene standards): terpineol (1), caryophyllene oxide (8), ocimene (12), γ-terpinene (15), β-pinene (19), 3-carene (21). TerpMixC (terpene standards): fenchol (no UV), camphene (16), α-phellandrene (17), α-humulene (27), β-caryophyllene (28). CB Std. (Cannabinoid standards): CBDVA (3), CBND (4), CBDV (5), CBDA (6), CBGA (7), CBG (9), CBD (10), THCV (11), CBN (20), Δ⁹-THC (23), Δ⁸-THC (24), THCA (25), CBC (26).

FIGS. 7A-7D illustrate the effect of cannabinoids on SOCE in Jurkat cells, in accordance with embodiments. Calcium signals are solicited in intact cells by applying 1 μM thapsigargin (Tg). Gadolinium (1 μM) was used as a positive control (pos ctl) of SOCE inhibition. All data are averages of three independent runs. FIG. 7A shows screening of seven THC derivatives, in accordance with embodiments. FIG. 7B shows screening of one high-THC extract, in accordance with embodiments. FIG. 7C shows screening of nine non-THC cannabinoids, in accordance with embodiments. FIG. 7D shows screening of one high-CBD extract, in accordance with embodiments.

FIGS. 8A-8D illustrates the effect of cannabinoids on SOCE in HEK293 cells, in accordance with embodiments. All data are averages of three independent runs. FIG. 8A shows screening of seven THC derivatives, in accordance with embodiments. FIG. 8B shows screening of one high-THC extract, in accordance with embodiments. FIG. 8C shows screening of non-THC cannabinoids, in accordance with embodiments. FIG. 8D shows screening of one high-CBD extract, in accordance with embodiments.

FIGS. 9A-9P illustrate dose-response behavior of cannabinoids on store-operated calcium entry (SOCE), in accordance with embodiments. All data are averages of three independent runs±SEM. FIG. 9A depicts dose-response behavior of CBGA, CBG, and a vehicle control. FIG. 9B depicts dose-response behavior of CBGVA, and CBGV. FIG. 9C depicts dose-response behavior of CBDA, CBD, CBDVA, and CBDV. FIG. 9D depicts dose-response behavior of CBCA, CBC, and CBCV. FIG. 9E depicts dose-response behavior of CBLA, and CBL. FIG. 9F depicts dose-response behavior of CBNA, CBN, CBND, and CBNM. FIG. 9G depicts dose-response behavior of THCA, delta9-THC, delta8-THC, and THCVA. FIG. 9H depicts dose-response behavior of CBGA, CBG, and a vehicle control. FIG. 9-I depicts dose-response behavior of CBGVA, and CBGV. FIG. 9J depicts dose-response behavior of CBDA, and CBD. FIG. 9K depicts dose-response behavior of CBCA, CBC, and CBCV. FIG. 9L depicts dose-response behavior of CBDVA, and CBDV. FIG. 9M depicts dose-response behavior of CBLA and CBL. FIG. 9N depicts dose-response behavior of CBNA, CBN, CBND, and CBNM. FIG. 9-0 depicts dose-response behavior of THCVA and THCV. FIG. 9P depicts dose-response behavior of THCA, delta8 THC, and delta9 THC.

FIGS. 10A-10Z illustrate combinatory effect of CBGA and other cannabinoids, in accordance with embodiments. FIGS. 10A-10T were obtained in Jurkat-NFAT cells and FIGS. 10U-10Y were obtained from THP-1 cells. All the data shown here are average of three independent runs and the values are mean±SEM. FIG. 10A depicts varying ratios of CBG to CBGA. FIG. 10B depicts varying ratios of CBGV to CBGA. FIG. 10C depicts varying ratios of THCVA to CBGA. FIG. 10D depicts varying ratios of THCV to CBGA. FIG. 10E depicts varying ratios of CBGVA to CBGA. FIG. 10F depicts varying ratios of THCA to CBGA. FIG. 10G depicts varying ratios of CBNA to CBGA. FIG. 10H depicts varying ratios of CBN to CBGA. FIG. 10-I depicts varying ratios of CBCA to CBGA. FIG. 10J depicts varying ratios of CBD to CBGA. FIG. 10K depicts varying ratios of CBND to CBGA. FIG. 10L depicts varying ratios of CBL to CBGA. FIG. 10M depicts varying ratios of CBDA to CBGA. FIG. 10N depicts varying ratios of CBDVA to CBGA. FIG. 10-0 depicts varying ratios of delta8 THC to CBGA. FIG. 10P depicts varying ratios of delta9 THC to CBGA. FIG. 10Q depicts varying ratios of CBDV to CBGA. FIG. 10R depicts varying ratios of CBLA to CBGA. FIG. 10S depicts varying ratios of CBC to CBGA. FIG. 10T depicts varying ratios of CBCV to CBGA. FIGS. 10U-10Y were obtained from THP-1 cells. FIG. 10U depicts varying ratios of CBDA to CBGA. FIG. 10V depicts varying ratios of CBGVA to CBGA. FIG. 10W depicts varying ratios of THCA to CBGA. FIG. 10X depicts varying ratios of THCVA to CBGA. FIG. 10Y depicts varying ratios of CBNA to CBGA. FIG. 10Z depicts % SOC inhibition for various ratios of cannabinoids and CBGA.

FIG. 11A-FIG. 11-SS show store-operated calcium entry (SOCE) dose response curves in human cells for various hemp extracts under heated or unheated conditions, in accordance with embodiments. FIGS. 11A-11-I depict store-operated calcium entry (SOCE) dose response curves in HEK293 cells for various hemp extracts under heated or unheated conditions.

FIG. 11A depicts dose response curves for hemp variety CW. FIG. 11B depicts dose response curves for hemp variety LIF. FIG. 11C depicts dose response curves for hemp variety WCBG. FIG. 11D depicts dose response curves for hemp variety ELEK. FIG. 11E depicts dose response curves for hemp variety SH. FIG. 11F depicts dose response curves for hemp variety SSC. FIG. 11G depicts dose response curves for hemp variety GS. FIG. 11H depicts dose response curves for hemp variety SS. FIG. 11-I depicts dose response curves for hemp variety HH.

FIGS. 11J-11R depict store-operated calcium entry (SOCE) dose response curves in Jurkat cells for various hemp extracts under heated or unheated conditions. FIG. 11J depicts dose response curves for hemp variety CW. FIG. 11K depicts dose response curves for hemp variety HH.

FIG. 11L depicts dose response curves for hemp variety SSC. FIG. 11M depicts dose response curves for hemp variety ELEK. FIG. 11N depicts dose response curves for hemp variety LIF. FIG. 11-O depicts dose response curves for hemp variety SS. FIG. 11P depicts dose response curves for hemp variety GS. FIG. 11Q depicts dose response curves for hemp variety SH. FIG. 11R depicts dose response curves for hemp variety WCBG.

FIGS. 11S-11AA depict store-operated calcium entry (SOCE) dose response curves in LUVA cells for various hemp extracts under heated or unheated conditions. FIG. 11S depicts dose response curves for hemp variety CW. FIG. 11T depicts dose response curves for hemp variety HH. FIG. 11U depicts dose response curves for hemp variety SSC. FIG. 11V depicts dose response curves for hemp variety ELEK. FIG. 11W depicts dose response curves for hemp variety LIF. FIG. 11X depicts dose response curves for hemp variety SS. FIG. 11Y depicts dose response curves for hemp variety GS. FIG. 11Z depicts dose response curves for hemp variety SH. FIG. 11AA depicts dose response curves for hemp variety WCBG.

FIGS. 11BB-11JJ depict store-operated calcium entry (SOCE) dose response curves in RBL2H3 cells for various hemp extracts under heated or unheated conditions. FIG. 11BB depicts dose response curves for hemp variety CW. FIG. 11CC depicts dose response curves for hemp variety HH. FIG. 11DD depicts dose response curves for hemp variety SSC. FIG. 11EE depicts dose response curves for hemp variety ELEK. FIG. 11FF depicts dose response curves for hemp variety LIF. FIG. 11GG depicts dose response curves for hemp variety SS. FIG. 11HH depicts dose response curves for hemp variety GS. FIG. 11-II depicts dose response curves for hemp variety SS. FIG. 11JJ depicts dose response curves for hemp variety WCBG.

FIGS. 11KK-11SS depict store-operated calcium entry (SOCE) dose response curves in U937 cells for various hemp extracts under heated or unheated conditions. FIG. 11KK depicts dose response curves for hemp variety CW. FIG. 11LL depicts dose response curves for hemp variety HH. FIG. 11MM depicts dose response curves for hemp variety SSC. FIG. 11NN depicts dose response curves for hemp variety ELEK. FIG. 11-OO depicts dose response curves for hemp variety LIF. FIG. 11PP depicts dose response curves for hemp variety SS. FIG. 11QQ depicts dose response curves for hemp variety GS. FIG. 11RR depicts dose response curves for hemp variety SH. FIG. 11SS depicts dose response curves for hemp variety WCBG.

FIG. 11TT-FIG. 11-XX show store-operated calcium entry (SOCE) dose response curves in human cells for various hemp extracts, in accordance with embodiments. FIG. 11TT depicts SOCE dose response curves with Jurkat cells. FIG. 11UU depicts SOCE dose response curves with Luva cells. FIG. 11VV depicts SOCE dose response curves with RBL2H3 cells.

FIG. 11-WW depicts SOCE dose response curves with U937 cells. FIG. 11-XX depicts SOCE dose response curves with HEK293 cells.

FIG. 12 illustrates the effect of CBGA in blocking Ca²⁺ Release-activated Ca²⁺ inward current (I_{CR}AC), in accordance with embodiments.

FIG. 13 illustrates the effect of CBGA on inward current and outward currents at −120 mV and +40 mV, respectively. CBGA blocks inward currents carried by Ca²⁺ Release-activated Ca²⁺ (CRAC) channels (gray symbols) in parallel with outward currents (black symbols) carried by TRPM7 (Transient receptor potential cation channel, subfamily M, member 7), in accordance with embodiments.

FIG. 14 illustrates activation of TRPM7 over-expressed in HEK293 cells by perfusing cell with intracellular solution containing 0 ATP and 0 Mg²⁺, resulting in fast and maximal activation of TRPM7 outward currents at +40 mV. CBGA dose-dependently inhibits TRPM7 currents, in accordance with embodiments.

FIG. 15 illustrates dose-response curves for the inhibition of TRPM7 currents (dark gray symbols) obtained in FIG. 14 and SOCE-mediated increases in intracellular Ca²⁺ (light gray symbols), in accordance with embodiments.

FIGS. 16A-16D illustrate the effect of whole-plant cannabis extracts on SOCE in Jurkat cells for samples S01-S04 extracted from the cannabis plant material, wherein the extracts were either tested as-is after extraction or heat treatment (h). FIG. 16A, S01; FIG. 16B, S02; FIG. 16C, S03; FIG. 16D, S04.

FIGS. 17A-17D illustrate the effect of whole-plant cannabis extracts on SOCE in Jurkat cells for samples S05-S08 extracted from cannabis plant material, wherein the extracts were either tested as-is after extraction or heat treatment (h). FIG. 17A, S05; FIG. 17B, S06; FIG. 17C, S07; FIG. 17D, S08.

FIGS. 18A-18C illustrate the effect of whole-plant cannabis extracts on SOCE in Jurkat cells for samples S09-S11 extracted from cannabis plant material, wherein the extracts were either tested as-is after extraction or heat treatment (h). FIG. 18A, S09; FIG. 18B, S10; FIG. 18C, S11.

FIGS. 19A-19B illustrate the effect of cannabis extracts on store-operated calcium entry (SOCE) in Jurkat cells for various samples extracted from cannabis plant material, and from hemp strains Otto-18, HAR (Harlequin), and BOAX. FIG. 19A shows SOCE data for extracts without heat treatment. FIG. 19B shows SOCE data for extracts with heat treatment.

FIG. 20 illustrates store-operated calcium entry (SOCE) signals in the presence of various cannabinoid extracts S01, S02, S05, and S07 at a concentration of 50 ug/mL which were either heated or unheated prior to testing. DMSO and gadolinium were used as negative and positive controls (ctls), respectively.

FIGS. 21A-21C illustrates store-operated calcium entry (SOCE) signals in the presence of various cannabinoid samples derived from Otto, HAR (Harlequin), and BOAX, which were either heated or unheated prior to testing. FIG. 21A, Otto; FIG. 21B, HAR (Harlequin); FIG. 21C, BOAX)

FIGS. 22A-22E illustrate store-operated calcium entry (SOCE) activity for hemp extracts with and without heat treatment and combined (1:1) unheated and heated hemp extracts in different cell types. FIG. 22A, Jurkat cells; FIG. 22B, RBL2H3 cells; FIG. 22C, U937 cells; FIG. 22D, Luva cells; FIG. 22E, HEK293 cells. SSC—Sour Space Candy; HH—Hawaiian Haze; SS—Special Sauce; SH—Suver Haze; WCBG—White CBG; ELEK—Elektra; CW—Cherry Wine; LIF—Lifter; GS—Grape Soda.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates to compositions comprising analgesic, anti-inflammatory, phytochemicals derived from the Cannabis plant and methods of treatment using the same. Cannabis sativa has two major classes of compounds: cannabinoid and terpenoid compounds. Terpenes represent one of the largest classes of natural products with greater than 55,000 known compounds and have a range of pharmacological properties that include anticancer, antimicrobial, antifungal, antiviral, antihyperglycemic, antiparasitic, anti-inflammatory, and analgesic effects. Similarly, cannabinoids have been reported to exhibit a wide range of biological effects, including some efficacy in the treatment of pain, chemotherapy-induced nausea and vomiting.

Cannabinoid drugs are presently used as analgesics, but experimental pain studies have produced mixed results and the analgesic properties of cannabinoids, particularly with respect to neuropathic pain remain controversial. The two main cannabinoids in Cannabis, the psychoactive Δ9-tetrahydrocannabinol (Δ9-THC) and the non-psychoactive cannabidiol (CBD), are both available in the United States as therapeutics. Marinol™ is a soft gelatin capsule containing Δ9-THC dissolved in sesame oil to treat nausea and vomiting associated with cancer chemotherapy in patients who have failed to respond adequately to conventional therapies. Epidiolex® is an oral solution comprising purified CBD for treating seizures associated with 2 rare forms of epilepsy—Dravet and Lennox-Gastaut Syndromes. Sativex® is a specific extract of Cannabis containing equal amounts of THC and CBD that was approved as a botanical drug in the United Kingdom in 2010 as a mouth spray to alleviate neuropathic pain, spasticity, overactive bladder, and other symptoms of multiple sclerosis.

The present disclosure describes high-throughput assays used to assess the efficacy and potency of various Cannabis phytochemicals—alone or in combination—in suppressing the pro-inflammatory activity of key immune cell types involved in inflammatory pain. The disclosure describes the characterization of relevant targets affected by various, non-hallucinatory, Cannabis phytochemicals and their analgesic properties in animal models of inflammatory nociceptive and neuropathic pain.

### Phytochemicals of the Cannabis Plant

Described herein are compositions present in the Cannabis plant (e.g., C. saliva). Such compounds may be categorized as cannabinoids. Exemplary cannabinoids, without limitation, are described in Table 1. In some embodiments, cannabinoids are extracted or otherwise obtained from plants such as Cannabis spp. (e.g., “plant based”). In some embodiments, cannabinoids are synthesized using chemical synthesis, recombinant biosynthesis, or a combination of both.

Cannabinoids in some instances comprise a diverse array of chemical functional groups or structural shapes which influence their biological activity. For example, acidic cannabinoids in some instances comprise at least one carboxylic acid group. Acidic cannabinoids include but are not limited to Cannabidivarinic Acid, Cannabigerovarinic acid, Cannabidiolic acid, Cannabigerolic acid, Tetrahydrocannabivarinic acid, Cannabinolic Acid, Tetrahydrocannabinolic Acid, Cannabichromenic Acid, or Cannabicyclolic Acid. In some embodiments, cannabinoids comprise one, two, three, or more than three chemical ring systems.

TABLE 1
describes exemplary cannabinoids.

| Cmpd | | | | |
|---|---|---|---|---|
| # | Abbrev. | Name | Structure | Chemical name |
| 1 | CBDVA | Cannabidivarinic Acid | 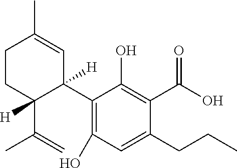 [SMILES: C=C(C)[C@@H]1CCC(C)=C[C@H]1c1c(O)cc(CCC)c(C(=O)O)c1O] | (1′R,2′R)-2,6-dihydroxy-5′- methyl-2′-(prop-1-en-2-yl)- 4-propyl-1′,2′,3′,4′- tetrahydro-[1,1′-biphenyl]- 3-carboxylic acid |
| 2 | CBND | cannabinodiol | 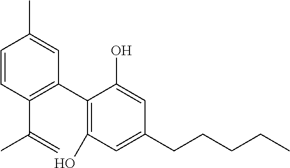 [SMILES: C=C(C)c1ccc(C)cc1-c1c(O)cc(CCCCC)cc1O] | 5′-methyl-4-pentyl-2′-(prop- 1-en-2-yl)-[1,1′-biphenyl]- 2,6-diol |
| 3 | CBGVA | Cannabigerovarinic acid | 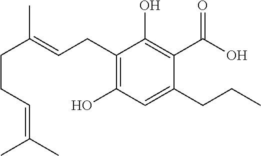 [SMILES: CCCc1cc(O)c(C/C=C(\C)CCC=C(C)C)c(O)c1C(=O)O] | (E)-3-(3,7-dimethylocta-2,6- dien-1-yl)-2,4-dihydroxy-6- propylbenzoic acid |
| 4 | CBDV | cannabidivarin | 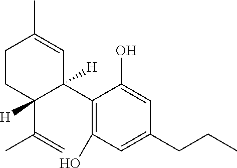 [SMILES: C=C(C)[C@@H]1CCC(C)=C[C@H]1c1c(O)cc(CCC)cc1O] | (1′R,2′R)-5′-methyl-2′- (prop-1-en-2-yl)-4-propyl- 1′,2′,3′,4′-tetrahydro-[1,1′- biphenyl]-2,6-diol |
| 5 | CBDA | Cannabidiolic acid | 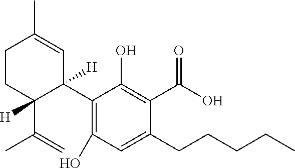 [SMILES: C=C(C)[C@@H]1CCC(C)=C[C@H]1c1c(O)cc(CCCCC)c(C(=O)O)c1O] | (1′R,2′R)-2,6-dihydroxy-5′- methyl-4-pentyl-2′-(prop-1- en-2-yl)-1′,2′,3′,4′- tetrahydro-[1,1′-biphenyl]- 3-carboxylic acid |
| 6 | CBGA | Cannabigerolic acid | 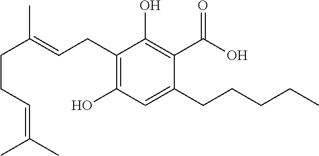 [SMILES: CCCCCc1cc(O)c(C/C=C(\C)CCC=C(C)C)c(O)c1C(=O)O] | (E)-3-(3,7-dimethylocta-2,6- dien-1-yl)-2,4-dihydroxy-6- pentylbenzoic acid |
| 7 | CBG | cannabigerol | 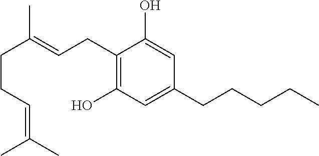 [SMILES: CCCCCc1cc(O)c(C/C=C(\C)CCC=C(C)C)c(O)c1] | (E)-2-(3,7-dimethylocta-2,6- dien-1-yl)-5-pentylbenzene- 1,3-diol |
| 8 | CBD | cannabidiol | 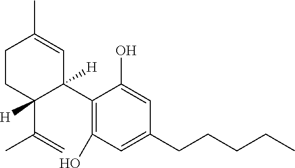 [SMILES: C=C(C)[C@@H]1CCC(C)=C[C@H]1c1c(O)cc(CCCCC)cc1O] | (1′R,2′R)-5′-methyl-4- pentyl-2′-(prop-1-en-2-yl)- 1′,2′,3′,4′-tetrahydro-[1,1′- biphenyl]-2,6-diol |
| 9 | THCV | Tetrahydrocannabivarin | 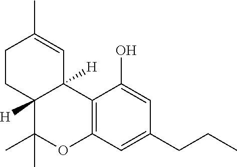 [SMILES: CCCc1cc(O)c2c(c1)OC(C)(C)[C@@H]1CCC(C)=C[C@@H]21] | (6aR,10aR)-6,6,9-trimethyl- 3-propyl-6a,7,8,10a- tetrahydro-6H- benzo[c]chromen-1-ol |
| 10 | CBCV | Cannabichromevarin | 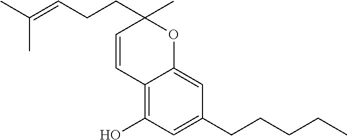 [SMILES: CCCCCc1cc(O)c2c(c1)OC(C)(CCC=C(C)C)C=C2] | 2-methyl-2-(4-methylpent- 3-en-1-yl)-7-pentyl-2H- chromen-5-ol |
| 11 | THCVA | Tetrahydrocannabivarinic acid | 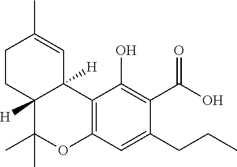 [SMILES: CCCc1cc2c(c(O)c1C(=O)O)[C@@H]1C=C(C)CC[C@H]1C(C)(C)O2] | (6aR,10aR)-1-hydroxy- 6,6,9-trimethyl-3-propyl- 6a,7,8,10a-tetrahydro-6H- benzo[c]chromene-2- carboxylic acid |
| 12 | CBN | cannabinol | 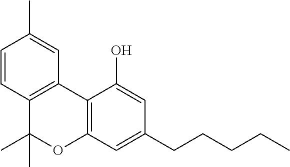 [SMILES: CCCCCc1cc(O)c2c(c1)OC(C)(C)c1ccc(C)cc1-2] | 6,6,9-trimethyl-3-pentyl- 6H-benzo[c]chromen-1-ol |
| 13 | CBNA | Cannabinolic Acid | 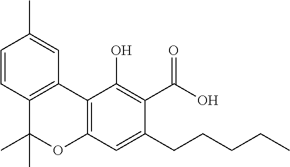 [SMILES: CCCCCc1cc2c(c(O)c1C(=O)O)-c1cc(C)ccc1C(C)(C)O2] | 1-hydroxy-6,6,9-trimethyl- 3-pentyl-6H- benzo[c]chromene-2- carboxylic acid |
| 14 | Δ9-THC | delta-9- tetrahydrocannabinol | 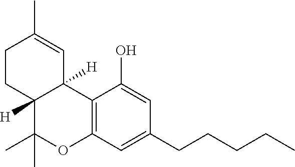 [SMILES: CCCCCc1cc(O)c2c(c1)OC(C)(C)[C@@H]1CCC(C)=C[C@@H]21] | (6aR,10aR)-6,6,9-trimethyl- 3-pentyl-6a,7,8,10a- tetrahydro-6H- benzo[c]chromen-1-ol |
| 15 | Δ8-THC | delta-8- tetrahydrocannabinol | 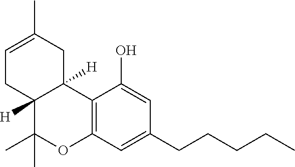 [SMILES: CCCCCc1cc(O)c2c(c1)OC(C)(C)[C@@H]1CC=C(C)C[C@@H]21] | (6aR,10aR)-6,6,9-trimethyl- 3-pentyl-6a,7,10,10a- tetrahydro-6H- benzo[c]chromen-1-ol |
| 16 | CBL | Cannabicyclol | 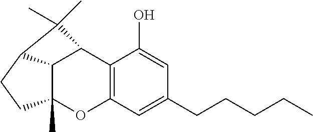 | (1aS,1a1R,3aR,8bR)-1,1,3a- trimethyl-6-pentyl- 1a,1a1,2,3,3a,8b-hexahydro- 1H-4- oxabenzo[f]cyclobuta[cd] inden-8-ol |
| 17 | CBC | cannabichromene | 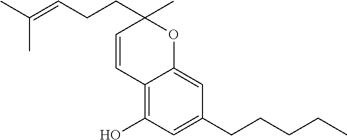 [SMILES: CCCCCc1cc(O)c2c(c1)OC(C)(CCC=C(C)C)C=C2] | 2-methyl-2-(4-methylpent- 3-en-1-yl)-7-pentyl-2H- chromen-5-ol |
| 18 | THCA | Tetrahydrocannabinolic Acid | 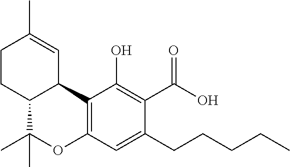 [SMILES: CCCCCc1cc2c(c(O)c1C(=O)O)[C@@H]1C=C(C)CC[C@H]1C(C)(C)O2] | (6aR,10aR)-1-hydroxy- 6,6,9-trimethyl-3-pentyl- 6a,7,8,10a-tetrahydro-6H- benzo[c]chromene-2- carboxylic acid |
| 19 | CBCA | Cannabichromenic Acid | 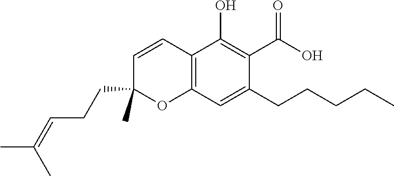 [SMILES: CCCCCc1cc2c(c(O)c1C(=O)O)C=C[C@@](C)(CCC=C(C)C)O2] | (R)-5-hydroxy-2-methyl-2- (4-methylpent-3-en-1-yl)-7- pentyl-2H-chromene-6- carboxylic acid |
| 20 | CBLA | Cannabicyclolic Acid | 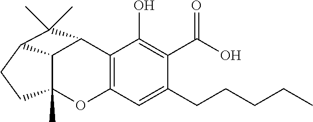 | (1aS,1a1R,3aR,8bR)-8- hydroxy-1,1,3a-trimethyl-6- pentyl-1a,1a1,2,3,3a,8b- hexahydro-1H-4- oxabenzo[f]cyclobuta[cd] indene-7-carboxylic acid |
| 21 | CBNM | Cannabinol methyl ether | 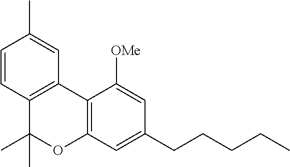 [SMILES: CCCCCc1cc(OC)c2c(c1)OC(C)(C)c1ccc(C)cc1-2] | 1-methoxy-6,6,9-trimethyl- 3-pentyl-6H- benzo[c]chromene |
| 22 | CBGV | Cannabigerovarin | 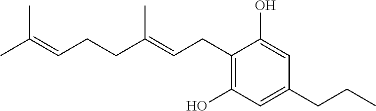 [SMILES: CCCc1cc(O)c(C/C=C(\C)CCC=C(C)C)c(O)c1] | (E)-2-(3,7-dimethylocta-2,6- dien-1-yl)-5-propylbenzene- 1,3-diol |
| 23 | 8β-OH- Δ9 THC | 8β-hydroxy-Δ9 tetrahydrocannabinol | 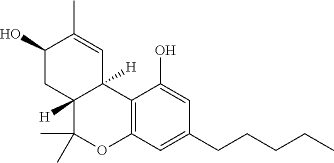 [SMILES: CCCCCc1cc(O)c2c(c1)OC(C)(C)[C@@H]1C[C@@H](O)C(C)=C[C@@H]21] | (6aR,8R,10aR)-6,6,9- trimethyl-3-pentyl- 6a,7,8,10a-tetrahydro-6H- benzo[c]chromene-1,8-diol |
| 24 | 11-OH- Δ9 THC | 11-hydroxy-Δ9 tetrahydrocannabinol | 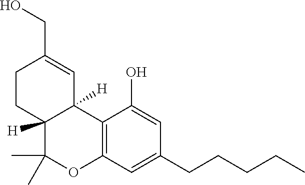 [SMILES: CCCCCc1cc(O)c2c(c1)OC(C)(C)[C@@H]1CCC(CO)=C[C@@H]21] | (6aR,10aR)-9- (hydroxymethyl)-6,6- dimethyl-3-pentyl- 6a,7,8,10a-tetrahydro-6H- benzo[c]chromen-1-ol |
| 25 | 11-COOH- Δ9-THC | 11-nor-9-carboxy-Δ9- tetrahydrocannabinol | 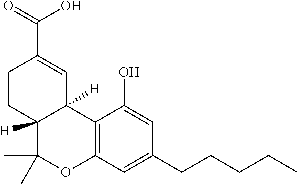 [SMILES: CCCCCc1cc(O)c2c(c1)OC(C)(C)[C@@H]1CCC(C(=O)O)=C[C@@H]21] | (6aR,10aR)-1-hydroxy-6,6- dimethyl-3-pentyl- 6a,7,8,10a-tetrahydro-6H- benzo[c]chromene-9- carboxylic acid |

### Sources of Cannabinoids

Extraction of Cannabinoids from Plant Source

Cannabinoids may be obtained as an extract from plant-based materials, such as Cannabis spp. or from organisms genetically modified to recombinantly synthesize them. The Cannabis spp. plant extract source may be plant material from regulated sources, for example, the National Institute on Drug Abuse (NIDA), or from hemp, (obtained from various vendors, including Berkshire CBD, Plain Jane, Earth Matters, Ventura Seed Company), which by definition comprises low to negligible levels of THC. Such extracts are in some instances used directly as pharmaceutical compositions. In some embodiments, extracts may be used as is or heated, as disclosed herein. In some embodiments, extracts are cold extracted. Methods for the extraction of cannabinoids from plant sources include but are not limited to supercritical fluid extraction, solvent extraction, ultrasound-assisted extraction, microwave-assisted extraction, or a combination thereof. In some embodiments, extraction of cannabinoids from plant sources comprises supercritical fluid extraction, solvent extraction, ultrasound-assisted extraction, microwave-assisted extraction, or a combination thereof. In some embodiments, a supercritical fluid comprises carbon dioxide, methane, ethane, propane, ethylene, or propylene. In some embodiments, extraction solvents comprise alcohols or non-polar solvents. In some embodiments, alcohols include methanol, ethanol, 2-propanol (isopropanol), n-butanol, tert-butanol, pentanol, or larger C₆-C₁₀ alcohols. In some embodiments, non-polar solvents comprise butane, pentane, hexane, heptane, cyclohexane, or larger C₅-C₁₂ alkanes. In some embodiments, the extraction product leads to less than 25%, 20%, 15%, 10%, 5%, 2%, 1%, or less than 0.5% decarboxylation of acidic cannabinoids. In some embodiments, the extraction product leads to 5-25%, 5-20%, 5-15%, 5-10%, 1-25%, 1-10%, 1-5%, or 0.5-1% decarboxylation of acidic cannabinoids. In some embodiments, the extraction product leads to at least 75%, 80%, 85%, 90%, 95%, 97%, 99%, or at least 99.5% decarboxylation of acidic cannabinoids. In some embodiments, the extraction product leads to 75-95%, 80-95%, 85-95%, 90-95%, 75-99%, 90-99%, 95-99%, or 99-100% decarboxylation of acidic cannabinoids. In some embodiments, extracts may be exposed to elevated temperatures, as disclosed herein.

Sources of cannabinoids include both Cannabis species, and strains thereof. In some embodiments, a cannabis plant is classified as Cannabis sativa or Cannabis indica, although in some classification references the genetic and phenotypic distinctions between Cannabis sativa and Cannabis indica are few, if any, and may better be referred to as strains, varieties or chemovars rather that bonafide species. See, e.g., Piomelli and Russo, “The Cannabis sativa versus Cannabis indica debate: An interview with Ethan Russo, MD” at https://www.ncbi.nlm.nih.gov/pmc/articles/PMC5576603/. Cannabis strains in some embodiments comprise a pure variety, or are generated by crossing two or more different varieties of Cannabis. In some embodiments, indica strains used herein include but are not limited to 3× Crazy, 8 Ball Kush, A-10, Abusive OG, Afghani, Afghani Bullrider, Afghan Kush, Afghan Skunk, Afgoo, Afgooey, Alien Dawg, Alien Kush, Ancient OG, Anesthesia, Aurora Indica, Banana Candy, Banana Kush, Barbara Bud, Berry Noir, Big Bud, Blackberry Hashplant, Blackberry Kush, Black Cherry Soda, Black Diamond, Black Label Kush, Blackwater, Blueberry, Blueberry Kush, Blueberry Waltz, Blue Cheese, Blue God, Blue Moonshine, Bubba Kush, Bubba OG, Buddha's Sister, Butterscotch, Candy Cane, Cactus, Cadillac Purple, Cheese Quake, Chem Scout, Cherry Kola, Chocolate Chunk, Church OG, Corleone Kush, Critical Mass, Danky Doodle, Dark Star, Darth Vader OG, Death Star, Death Star OG, Deep Purple, Denver Maple, Devil Fruit, Diablo, Diamond OG, Digweed, DJ Short Blueberry, Domino, Dynamite, Edelweiss, El Jeffe, Enigma, Endless Sky, Eran Almog, Freezeland, Frosty, Fucking Incredible, Funky Monkey, G13, Godberry, God Bud, Godfather OG, God's Gift, Gog & Magog, Granddaddy Purple, Grand Hindu, Grape Ape, Grape Crush, Grape Skunk, Gravity, Green Crack, G Stik “RED”, G Stik “Violeta”, Guido Kush, Gumbo, Haoma, Hashberry, Hash Plant, Hawaiian Delight, Herojuana, Hindu Kush, Hog, Hog's Breath, Hollands Hope, Ingrid, Jagermeister, Jesus OG, JT15, Jupiter Kush, Jupiter OG, K Train, Kandahar, King Kush, King Louis, Kosher Kush, Kryptonite, Kushadelic, Kush Berry, LA OG, Larry OG, Lavender Kush, Lemon OG, Lions Gate, Liquid Butter, Louis XIII, Madagascar, Mango, Maple Leaf Indica, Mars OG, Master Bubba, Master Jedi, Master Kush, Matsu, Mazar I Sharif, Mercury OG, Misty Kush, MK Ultra, Mossimo OG, Mr. Nice, Negra 44, Neptune Kush, Neptune OG, Nordle, Northern Lights, Nuggetry OG, Obama Kush, OG Eddy Lepp, Paris OG, Pennywise, Pineapple Thai, Pitbull, Platinum Bubba, Platinum Bubba Kush, Platinum Kush, Platinum OG, Platinum Purple Kush, Plushberry, Pluto Kush, Popcorn Kush, Pot of Gold, Power Kush, Presidential OG, Pure Kush, Pure OG, Purple Afghani, Purple Berry, Purple Candy, Purple Cream, Purple Dragon, Purple Goo, Purple Kush, Purple Mr. Nice, Purple Nepal, Purple OG Kush, Purple Passion, Purple Tonic, Purple Urkle, Purple Wreck, Pre-98 Bubba Kush, PVC OG, Raspberry Kush, Red Dwarf, Red Eye OG, Redwood Kush, Remedy, Rockstar, Rockstar Master Kush, Romulan, Seattle Blue, Sensi Star, Shark Shock, Silverback Gorilla, Skywalker, Snow Monster, Somango, South Central LA, Space Dawg, Strawberry Kush, Sunshine Daydream, Super Kush, Superman OG, Super Skunk, Tahoe OG, Taliban Poison, Tangerine Kush, True OG, UW, Vanilla Kush, Violator Kush, White Kush, White OG, Wonder Woman OG, Woody Kush, XXX 420, XXX OG, Yoda OG, Yumboldt, and Zombie OG. In some embodiments, sativa strains used herein include but are not limited to Aceh, Alaskan Thunder Fuck, Alice in Wonderland, Allen Wrench, Aloha, Amnesia, Amnesia Haze, Arabian Gold, Arjan's Strawberry Haze, Asian Fantasy, Bay 11, Berkely, Black Diesel, Blue Bayou, Blue Satellite, Brainstorm Haze, Candy Jack, Cannalope Haze, Cat Piss, Charlotte's Web, Chocolope, Cracker Jack, Crystal Coma, Django, Double Diesel, Dr. Grinspoon, Durban Poison, Dutch Dragon, Dutch Hawaiian, East Coast Sour Diesel, Fire Haze, Frostbite, Ghost Train Haze, Goldwing, Grapefruit, Green Candy, Green Crack, Green Crack Extreme, Green Goblin, Green Haze, G Stik “Amarillo”, G Stik “Rosado”, Hawaiian, Hawaiian Diesel, Hawaiian Sativa, Hawaiian Snow, Haze, Haze Wreck, Hempstar, Hydro Green Crack, Island Sweet Skunk, J-27, Jack Herer, Jack Skellington, Jack Wreck, Jamaican Pearl, Jean Guy, Johnny's Tonic, Kaboom, Kali Mist, Killing Fields, King's Bread, Lamb's Bread, Laughing Buddha, Lemon Haze, Lemon Jack, Lemon G, Lemon Sativa, Lemon Thai, Mako Haze, Malawi, Mango Dream, Maui, Maui Waui, Mexican Sativa, Moby Dick, Mother's Finest, Nevil's Wreck, OCD, OG, Outer Space, Panama Red, Pandora's Box, Poison Haze, Purple Haze, Q3, Qleaner, Rafael, Red Congolese, Red Haze, Rene, Root Beer Kush, Sage N Sour, Shaman, Shangri La OG, Shipwreck, Silver Haze, Skunk #1, Sour Chocolate, Sour Diesel, Sour Flower, Sour Haze, Sour Tangie, Strawberry, Sugar Plum, Super Cat Piss, Super Green Crack, Super Silver Haze, Super Snow Dog, Super Sour Diesel, Sweet Diesel, Tangie, Thai, Thai Haze, Timewreck, Triple Diesel, Twista, Ultimate Trainwreck, Venice OG, Very Berry Haze, Voodoo, Vortex, Waipi'o Hapa, Willie Nelson, Willy Wonka, and Y Griega. In some embodiments, indica-dominant hybrid strains used herein include but are not limited to 303 OG, 707 Headband, Afghanica, AK-48, Albert Walker, BC Sweet Tooth, Big Sky OG, Big Wreck, Bio-Jesus, Black Domina, Black Ice, Black Mamba, Blockhead, Blue Bastard, Blueberry×Hash Plant, Blueberry Diesel, Blue Diesel, Blue Ivy, Blue Lights, Blue Magoo, Blue Venom, Burmese Kush, Cali Gold, Chemdawg 4, Citrus Kush, Cold Creek Kush, Confidential Cheese, Cotton Candy Kush, Critical Bilbo, Critical Haze, Dorit, Dr. Who, Early Girl, Elderberry Kush, Erez, Flowerbomb Kush, God's Treat, Hells Angel OG, Ice Cream, Jasmin or Yasmin, Lifesaver, Little Devil, LVPK, Manitoba Poison, Master Skunk, Matanuska Thunder Fuck, Millennium, Or, Papaya, Pineapple Chunk, Pink Kush, Pink Pez, Rascal OG, Shiva Skunk, Skywalker OG, Snoop's Dream, Sour Apple, Sugar Shack, Sunset Sherbert, Supernova, The White, Tres Dawg, Venom OG, Warlock, and White Lightning. In some embodiments, sativa-dominant hybrid strains used herein include but are not limited to ACDC, Afwreck, Alaska, Avi-Dekel, Banana Diesel, Belladonna, Blue Champagne, Blue Diamond, Blue Dream, Blue Goo, Blue Haze, Candyland, Cinderella 99, Double Dream, Dream Star, Duke Nukem, Floppy Donged Gorilla, Fruit Spirit, Glass Slipper, Green Dream, Hashplant Haze, Hawaiian Fire, Hawaiian Skunk, Headbanger, Head Cheese, Hippie Crippler, Hong Kong, Huckleberry, Humboldt, J1, Jack Haze, Jack's Cleaner, Jack Widow, Jane Doe, Juliet, KI, Lemon Kush, Lemon Wreck, Lethal Purple, Loud Dream, Midnight, Mob Boss, NYC Diesel, OG Wreck, Orange Dream, Orange Haze, Pineapple Diesel, Power Plant, Pure Power Plant, Purple Ice, Sapphire Star, Silver Pearl, Silver Surfer, Smelliot, Snowcap, Sour Cream, Sour Lemon OG, Sour Power, Sour Tsunami, Stevie Wonder, Strawberry Cough, Strawberry Dream, Sumatra Kush, Superstar, Sweet Island Skunk, Swiss Bliss, Tahoe OG Kush, Tangerine Haze, Thai-Tanic, The Third Dimension, The Truth, Trainwreck, White Berry, White Fire OG, and White Shark. In some embodiments, hybrid strains used herein include but are not limited to $100 OG, 3 Kings, Ace of Spades, Afghan Big Bud, Afghan Diesel, Afghooey, Agent Orange, AK-47, Alien OG, Alohaberry, Alpha OG, Ambrosia, Animal Cookies, Apollo 11, Apollo 13, Appalachia, Armageddon, Atomic Northern Lights, A-Train, Avalon, Banana OG, BC Roadkill, Berry White, Big Buddha Cheese, Bio-Diesel, Black Dahlia, Black Jack, Black Tuna, Black Velvet, Black Widow, Blueberry Cheesecake, Blueberry Haze, Blueberry Headband, Blue Boy, Blue Dragon, Blue Hawaiian, Blue Mystic, Blue OG, Blue Rhino, Blue Train, Blue Widow, Boggle Gum, Bordello, Boysenberry, Brains Damage, Broke Diesel, Bruce Banner #3, Bubbleberry, Bubblegum, Bubblegun, C13 Haze, Cali Kush, Calm, Cannadential, Cannatonic, Caramelicious, Casey Jones, Cataract Kush, Champagne Kush, Cheese, Chem Crush, Chemdawg, Chernobyl, Cherry AK-47, Cherry Bomb, Cherry Kush, Cherry Limeade, Cherry Pie, Chiesel, Chocolate Kush, Cinex, Colorado Chem, Connie Chung, Cookies and Cream, Crimea Blue, Dairy Queen, Deadhead OG, Diesel Duff, Dieseltonic, Dogwalker OG, Donna OG, Dopium, Dreadlock, Dream Queen, Durban Cheese, Dutch Treat, Euphoria, Earth OG, Earthquake, Ed Rosenthal Super Bud, El-Na, El Nino, Ewok, Exodus Cheese, Fire OG, Flourish, Fruity Pebbles, Ghost OG, Girl Scout Cookies, Goldberry, Golden Goat, Gorilla Glue #4, Grand Hustle, Grapefruit Diesel, Grapefruit Haze, Grapefruit Kush, Grape God, Grape Kush, Green Door Kush, Green Hornet, Green Monster, Green Queen, Green Ribbon, G Stik “Orange”, G Stik “Verde”, Guava Chem, Guava Kush, Harlequin, Harmony, Hawaiian Punch, Hawaiian Thunder Fuck, Headband, Heavy Duty Fruity, Himalayan Gold, Hindu Skunk, Holistic, Holy Grail Kush, Ice, Jah Kush, Jack Flash, Jack Frost, Jack Kush, Jack the Ripper, Jilly Bean, Juicy Fruit, Julius Caesar, Kahuna, Key Lime Haze, Khola, Killer Queen, Kimbo Kush, King Kong, Kushage, Kushashima, Kushberry, LA Jack, Lambo OG, LAPD, LA Woman, Lavender, Lemon Diesel, Lemon Drop, Lemon Skunk, Lemon Walker OG, Liberty Haze, Lowryder, LSD, Lucky Charms, M-39, Mad Dog, Mango Kush, Maui Berry, Martian Mean Green, Master Yoda, Medicine Man, Mendocino Purps, Mother's Helper, Nebula, Neville's Haze, Northern Lights #5, Northern Skunk, Odyssey, OG Diesel Kush, OGiesel, OG Kush, OG Poison, OG Shark, Old Lyme Connecticut OG, Orange Bud, Orange Crush, Orange Diesel, Orange Kush, Orange Skunk, Orange Velvet, Organic Diesel, P-91, Permafrost, Pineapple, Pineapple Express, Pineapple Kush, Platinum Girl Scout Cookies, Purple AK-47, Purple Arrow, Purple Diesel, Purple Dream, Purple Hashplant, Purple Jack, Purple Princess, Purple Sage, Purple Sour Diesel, Purple Trainwreck, Purple Voodoo, Qrazy Train, Quebec Gold, Querkle, Rainbow, Recon, Red Cherry Berry, Redding OG, Richie Rich, Rocklock, Romping Goddess, Romulan Grapefruit, Royal Dwarf, Royal Kush, Russian Rocket Fuel, SAGE, Saturn OG, SFV OG, Sharksbreath, Shiatsu Kush, Shishkaberry, Shoreline, Sierra Mist, Skunky Diesel, Sky Dog, Snow White, Sour Cheese, Sour Dream, Sour Grape, Sour Grapes, Sour Jilly, Sour Kush, Sour OG, Space Bomb, Space Queen, Stardawg, Starry Night, Strawberry Banana, Strawberry Diesel, Super Blue Dream, Super Lemon Haze, Super Sour Widow, Sweet Kush, Sweet Tooth, Tangerine Dream, Thaidal Wave, THC Bomb, The Flav, The Void, Thin Mint Girl Scout Cookies, Tiger Woods, Tranquil, Trident, Trifecta, Tropicali, Tropical Tang, UK Cheese, Unwind, Verde Electric, Waldo, Walrus Kush, White Dawg, White Fire Alien OG, White Queen, White Rhino, White Russian, White Widow, White Zombie, Willy's Wonder, Wonder Haze, Wonder Woman, Wonka's Bubblicious, and XJ-13.

In some instances, the source of cannabinoids is hemp. In some instances, the hemp comprises CBG-dominant varieties such as StemCell, CBGenius, or WhiteCBG. In other instances, the hemp strain comprises CBD-dominant varieties that include but are not limited to Otto-18, HAR (harlequin), BOAX, Cherry Wine, Silver Haze, Lifter, Elektra, Sour Space Candy, Special Sauce, Hawaiian Haze, or Grape Soda. In some instances, the hemp strain may be chosen from known CBG-dominant and/or CBD-dominant varieties. See, e.g., https://www.kyagr.com/marketing/documents/HEMP_LH_Summary_of_Varieties_List_2019.p df.

In some embodiments, additional components are added to such extracts. In some embodiments, cannabinoid extracts are mixed together. In some embodiments, cannabinoid extracts from the same cannabinoid source are mixed together. In some embodiments, cannabinoid extracts from at least two different cannabinoid sources are mixed together. In some embodiments, the cannabinoid source is hemp. In some embodiments, the cannabinoid extract in the mixture is heated and cannabinoids decarboxylated. In yet other embodiments, the cannabinoid extract in the mixture is not decarboxylated. Cannabinoids and extracts thereof may be combined with additional components. In some embodiments, additional components are added to such extracts.

In some embodiments, extracts comprise increased amounts of desired cannabinoids (e.g., cannabidiolic acid), and decreased amounts of undesired cannabinoids, or other impurity. Amounts of impurities may be measured by any method known in the art. In some embodiments, the amount of impurities is measured using HPLC, GC, GC/MS, NMR or other analytical method. The commercial standards of the cannabinoids, terpenes, flavonoids and other phytochemicals of Cannabis spp are obtained from various chemical vendors, including Cayman Chemical Company, Sigma-Aldrich, NIDA, etc. In some embodiments, purity is measured against a standard sample of known purity. In some embodiments, extracts comprise at most 50%, 45%, 40%, 35%, 30%, 25%, 20%, 15%, 10%, or at most 5% (w/w) impurities. In some embodiments, extracts comprise about 50%, 45%, 40%, 35%, 30%, 25%, 20%, 15%, 10%, or about 5% (w/w) impurities. In some embodiments, extracts comprise 1-2%, 1-5%, 1-15%, 2-10%, 2-15%, 5-10%, 5-20%, 10-25%, or 5-25% (w/w) impurities.

Extracts may comprise one or more cannabinoids. In some embodiments, extracts comprise no more than 99%, 98%, 95%, 90%, 85%, 80%, 75%, 70%, 65%, 60%, 55%, or no more than 50% cannabinoids. In some embodiments, extracts comprise no more than 99%, 98%, 95%, 90%, 85%, 80%, 75%, 70%, 65%, 60%, 55%, or no more than 50% cannabidiolic acid (CBDA) or cannabigerolic acid (CBGA). In some embodiments, extracts comprise 50-99%, 50-98%, 50-95%, 50-90%, 50-85%, 20-95%, 30-90%, 50-80%, or 50-50% cannabidiolic acid (CBDA) or cannabigerolic acid (CBGA). In some embodiments, extracts comprise no more than 99%, 98%, 95%, 90%, 85%, 80%, 75%, 70%, 65%, 60%, 55%, or no more than 50% cannabidiolic acid (CBDA) and cannabigerolic acid (CBGA). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), or cannabigerol (CBG). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), and cannabigerol (CBG). In some embodiments, extracts comprise 50-99%, 50-98%, 50-95%, 50-90%, 50-85%, 20-95%, 30-90%, 50-80%, or 50-50% cannabidiolic acid (CBDA), cannabigerolic acid (CBGA), and cannabigerol (CBG). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), cannabigerol (CBG), or cannabidiol (CBD). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), cannabigerol (CBG), and cannabidiol (CBD). In some embodiments, extracts comprise 50-99%, 50-98%, 50-95%, 50-90%, 50-85%, 20-95%, 30-90%, 50-80%, or 50-50% cannabidiolic acid (CBDA), cannabigerolic acid (CBGA), cannabigerol (CBG), and cannabidiol (CBD).

Extracts may comprise one or more additional impurities. Such impurities include but are not limited to non-cannabinoid terpenes, flavonoids, lignans, or other cannabinoids. In some embodiments, terpenes comprise camphene, 3-carene, β-caryophyllene, caryophyllene oxide, fenchol, β-myrcene, α-humulene, limonene, linalool, ocimene, α-phellandrene, α-pinene, β-pinene, terpineol, γ-terpinene, or terpinolene. In some embodiments, flavonoids comprise apigenin, cannflavin A, cannflavin B, kaempferol, luteolin, orientin, quercetin, or vitexin. In some embodiments, lignans comprise cannabisin A, cannabisin B, cannabisin D, cannabisin F, N-trans-caffeoyltyramine, N-trans-coumaroyltyramine, or N-trans-feruloyltyramine. In some embodiments, cannabinoid impurities comprise at least one of cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahydrocannabinol (Δ8-THC), cannabicyclol (CBL), cannabichromene (CBC), tetrahydrocannabinolic acid (THCA), cannabichromenic acid (CBLA), cannabinol methyl ether (CBNM). In some embodiments, cannabinoid impurities comprise at least two of cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahydrocannabinol (Δ8-THC), cannabicyclol (CBL), cannabichromene (CBC), tetrahydrocannabinolic acid (THCA), cannabichromenic acid (CBLA), cannabinol methyl ether (CBNM). In some instances, compositions described herein comprise no more than 1, 2, 3, 4, 5, 6, 7, or 8 impurities.

Extracted cannabinoids may be purified to a known purity. In some embodiments, the purified extracted cannabinoids are cannabidiolic acid (CBDA) or cannabigerolic acid (CBGA). In some embodiments, the extracted cannabinoids are purified such that it comprises at least 50%, 55%, 60%, 65%, 70%, 75%, 80%, 85%, 90%, 91%, 92%, 93%, 94%, 95%, 96%, 97%, 98%, 99% or at least 99.5% (w/w) of the desired cannabinoid. In some embodiments, the extracted cannabinoids are purified such that it comprises no more than 50%, 45%, 40%, 35%, 30%, 25%, 20%, 15%, 10%, or about 5% (w/w) of other cannabinoids. In some embodiments, extracts comprise about 1-2%, 1-5%, 1-15%, 2-10%, 2-15%, 5-10%, 5-20%, 10-25%, or 5-25% (w/w) of other cannabinoids.

Chemical or Biological Synthesis

Compositions described herein may comprise cannabinoids created synthetically (e.g., “synthetic” cannabinoids). Such synthesis methods include chemical synthesis or biological synthesis (e.g., recombinant expression of biosynthetic pathways). In some embodiments, cannabinoids are generated using a combination of chemical and biosynthetic methods (e.g., semi-synthesis). Chemical methods of cannabinoid synthesis are described in Shultz et al. Org. Lett. 2018, 20, 2381-384, and references cited therein. In some embodiments, cannabinoids are recombinantly expressed in a host organism such as a eukaryote or prokaryote. In some embodiments, cannabinoids are recombinantly expressed in a host organism such as a eukaryote cell or prokaryote cell. In some embodiments the host organism is a non-cannabis plant, such as a tobacco plant or an insect cell. In some the host organism is a microorganism. In some the host organism is yeast. In some the host organism is E. coli. In some embodiments the host organism is not a human. Recombinant methods of cannabinoid synthesis are described in Carvalho et al. FEMS Yeast Res. 2017, 17(4), 1, and references cited therein.

### Temperature Lability

Compositions described herein may comprise temperature labile compounds, wherein exposure to heat or elevated temperature causes structural changes in the compounds. In some instances, control of temperature during processing of compositions (e.g., extraction or other process) influences the chemical composition of the resulting extract or product. Structural changes variously comprise isomerization of bonds, elimination reactions, substitution, ring formation, ring opening, or other chemical reactions. The rate of change and amount of temperature modified product for such compounds in some instances depends on both temperature and time the compound is exposed to a given temperature. In some instances, compositions or compounds are treated with heat to effect chemical changes in the compounds thereof. Such changes in some embodiments increase the amount of desired compounds and/or decrease the amount of undesired compounds.

In some embodiments, processes are conducted at a temperature of less than 120, 110, 100, 90, 80, 70, 60, 55, 50, 45, 40, 35, 30, 25, 20, 15, 10, or less than 5° C. In some embodiments, processes are conducted at a temperature of about 120, 110, 100, 90, 80, 70, 60, 55, 50, 45, 40, 35, 30, 25, 20, 15, 10, or less than 5° C. In some embodiments, processes are conducted at a temperature of about 90, 80, 70, 60, 55, 50, 45, 40, 35, 30, 25, 20, 15, 10, or less than 5° C. In some embodiments, processes are conducted at a temperature of 100-120, 75-120, 10-120, 10-110, 10-100, 10-90, 20-80, 30-70, 40-60, 20-60, 30-50, 25-50, 10-45, 10-50, 20-50, 20-45, or 5-50° C. In some embodiments, extracts comprising one or more of CBGA, CBGVA, THCA, THCVA, CBDA, CBDVA, CBCA, and/or CBCVA is heated. In some embodiments, heat treatment of such an extract results in enrichment of THC, THCV, CBD, CBDV, CBC, CBCV, CBG, and/or CBGV. For example, decarboxylation reactions in some embodiments convert acidic cannabinoids into other cannabinoids. In some instances, decarboxylation results in conversion of THCA to THC, CBDA to CBD, or CBGA to CBG. In some embodiments, a composition is exposed to an elevated temperature by heating for at least 1, 2, 5, 10, 12, 15, 20, 30, 45, or at least 60 seconds. In some embodiments, a composition is exposed to an elevated temperature (such as above 20° C.) by heating for at least 1, 2, 5, 10, 12, 15, 20, 30, 45, or at least 60 minutes. In some embodiments, a composition is exposed to an elevated temperature by heating for at least 1, 2, 5, 10, 12, 15, 20, 30, 45, or at least 60 hours. In some embodiments, a composition is exposed to an elevated temperature by heating for 1-5, 1-10, 2-5, 2-10, 8-15, 10-20, 10-15, 20-30, 20-45, or 30-60 seconds. In some embodiments, a composition is exposed to an elevated temperature by heating for 1-2, 1-5, 1-10, 2-5, 2-10, 8-15, 10-20, 10-15, 20-30, 20-45, or 30-60 minutes. In some embodiments, a composition is exposed to an elevated temperature by heating for 1-2, 1-5, 1-10, 2-5, 2-10, 8-15, 10-20, 10-15, 20-30, 20-45, or 30-60 hours. In some embodiments, a composition is heated for 25-45 minutes at 100-120° C. In some embodiments, a composition is heated for 25-60 minutes at 100-120° C. In some embodiments, a composition is heated for 5-60 minutes at 100-120° C. In some embodiments, a composition is heated for 10-60 minutes at 100-120° C. In some embodiments, a composition is heated for 25-60 minutes at 20-120° C. In some embodiments, a composition is heated for 25-45 minutes at 120-140° C. In some embodiments, a composition is heated for 25-45 minutes at 140-160° C. In some embodiments, a composition is heated for 45 minutes to 5 hours at 90-120° C. In some embodiments, a composition is heated for 6-12 minutes at 120-140° C. In some embodiments, a composition is heated for 3-9 minutes at 135-155° C.

### Synergistic Combinations

Described herein are compositions, such as pharmaceutical compositions comprising two or more chemical compounds. In some embodiments, a first chemical compound is a cannabinoid. In some embodiments, a first chemical compound and a second chemical compound are each cannabinoids. In some embodiments, the first cannabinoid is an acidic cannabinoid. Combinations of two or more cannabinoids in some embodiments produce additive, sub-additive, supra-additive, or entourage biological effects. In some embodiments an additive effect is measured by combination indices (CI) according to the method of isoboles. In some embodiments a supra-additive effect is measured by combination indices (CI) according to the method of isoboles.

A composition described herein may comprise at least a first cannabinoid and a second cannabinoid. In some embodiments, the first cannabinoid is an acidic cannabinoid. In some embodiments, the first cannabinoid is Cannabidivarinic Acid, Cannabigerovarinic acid, Cannabidiolic acid, Cannabigerolic acid, Tetrahydrocannabivarinic acid, Cannabinolic Acid, Tetrahydrocannabinolic Acid, Cannabichromenic Acid, or Cannabicyclolic Acid. In some embodiments, the first cannabinoid is an acidic cannabinoid. In some embodiments, the first cannabinoid is Cannabigerolic acid (CBGA). In some embodiments, the second cannabinoid is an acidic cannabinoid. In some embodiments, the second cannabinoid is a non-acidic cannabinoid. In some embodiments, the second cannabinoid is cannabidiolic acid (CBDA), cannabidivarin (CBDV), cannabigerol (CBG), cannabidiol (CBD), tetrahydrocannabinolic acid (THCA), cannabigerovarinic acid (CBGVA), or tetrahydrocannabivarinic acid (THCVA). In some embodiments, the second cannabinoid is cannabigerol (CBG), cannabidiol (CBD), cannabidivarin (CBDV), or tetrahydrocannabinol (THC).

Compositions comprising two or more cannabinoids may be present in a variety of ratios. In some embodiments, a first cannabinoid is cannabigerolic acid (CBGA). In some embodiments, a second cannabinoid is cannabigerol (CBG), cannabidiol (CBD), cannabidivarin (CBDV), or tetrahydrocannabinol (THC). Such ratios are described in terms of mole ratio or in terms of a weight ratio. In some embodiments, the mole ratio of a first cannabinoid to a second cannabinoid is about 200:1, 100:1, 50:1, 25:1, 20:1, 15:1, 10:1, 5:1, 2:1, or 1:1. In some embodiments, the mole ratio of a first cannabinoid to a second cannabinoid is at least 100:1, 50:1, 25:1, 20:1, 15:1, 10:1, 5:1, 2:1, or 1:1. In some embodiments, the mass ratio of a first cannabinoid to a second cannabinoid is about 200:1, 100:1, 50:1, 25:1, 20:1, 15:1, 10:1, 5:1, 2:1, or 1:1. In some embodiments, the mass ratio of a first cannabinoid to a second cannabinoid is at least 200:1, 100:1, 50:1, 25:1, 20:1, 15:1, 10:1, 5:1, 2:1, or 1:1. In some embodiments, the mass ratio of a first cannabinoid to a second cannabinoid is 200-1:1:, 100:1-1:1, 50:1-1:1, 25:1-1:1, 100:1-50:1, 50:1-10:1, 20:1-1:1, 10:1-1.5:1.

### Inhibitory Compounds

Compositions described herein may have one or more effects on cells. In some embodiments, the cells comprise immune cells. Immune cells in some instances comprise lymphocytes, monocytes, neutrophils, leukocytes, phagocytes, macrophages, microglia, mast cells, or other immune cells. Immune cells in some instances comprise lymphocytes, monocytes, macrophages, microglia, or mast cell. Lymphocytes include but are not limited to T-cells, B-cells, NK-cells, helper T-cells, cytotoxic T lymphocytes. In some embodiments, the effect comprises an inhibitory effect on one or more cellular processes in such cells. In some embodiments, the cellular process comprises activation of one or more immune cells. Without being bound by theory, compositions described herein modulate calcium influx in immune cells. In some embodiments, inhibition or modulation by the compositions described herein may result in a decrease in adverse side effects in a patient treated therein.

In some embodiments, modulation comprises inhibition of calcium channels. In some embodiments, inhibition of calcium influx comprises the mechanism of Store-Operated Calcium Entry. In some embodiments, the cellular process comprises secretion of cytokines or chemokines. In some embodiments, secretion of cytokines is inhibited in two or more immune cells. In some embodiments, the cytokines comprise those involved in inflammation. In some embodiments, the secretion of two or more cytokines is inhibited. In yet other embodiments, the inhibition of cytokine secretion manifests in a decrease in adverse effects. Exemplary cytokines include but are not limited to interleukin-1 (IL), including but not limited to IL-1, IL-2, IL-4, IL-5, IL-6, IL-8, ILL-10, IL-12, and IL-18, tumor necrosis factor alpha (TNF-α), interferon gamma (IFNγ), granulocyte-macrophage colony stimulating factor (GM-CSF), and combinations thereof.

An inhibitory effect may be measured by a percent inhibition relative to cells without treatment using the compositions described herein. In some embodiments, secretion of at least one cytokine (e.g., inflammatory cytokine) is reduced by about 5%, 10%, 20%, 30%, 40%, 50%, 75%, 80%, 90%, 100%. In some embodiments, secretion of at least one cytokine (e.g., inflammatory cytokine) is reduced by at least 5%, 10%, 20%, 30%, 50%, 60%, 75%, 80%, 90% or at least 100%. In some embodiments, secretion of at least one cytokine (e.g., inflammatory cytokine) is reduced by 5-25%, 20-100%, 30-100%, 15-75%, 90-100%, 50-100%, or 40-100%.

In some embodiments, secretion of at least one cytokine (e.g., inflammatory cytokine) is reduced by about 1.2, 1.5, 2, 2.5, 5, 10, 15, 20, 50, 100, 200, 300, 500, or about 1000 fold. In some embodiments, secretion of at least one cytokine (e.g., inflammatory cytokine) is reduced by at least 1.2, 1.5, 2, 2.5, 5, 10, 15, 20, 50, 100, 200, 300, 500, or at least 1000 fold. In some embodiments, secretion of at least one cytokine (e.g., inflammatory cytokine) is reduced by 1.2-1000, 2-1000, 5-1000, 10-1000, 50-1000, 100-1000, 200-1000, 5-500, 5-100, 50-500, 100-500 fold.

An inhibitory effect may be measured as a degree of inhibition, defined as the ratio of the rate in the absence of inhibitor vₒ vs. the rate in the presence of inhibitor vi. An inhibitory effect may be measured by half the concentration of drug needed to achieve inhibition of the target (IC₅₀). In some embodiments, the composition described herein inhibits release of a cytokine (e.g. inflammatory cytokine) with an IC₅₀ of about 100 μM, 50 μM, 25 μM, 10 PM, 5 μM, 1 μM, 500 nM, 250 nM, 200 nM, 150 nM, 100 nM, 50 nM, 25 nM, 10 nM, 5 nM, or about 1 nM. In some embodiments, the composition described herein inhibits release of a cytokine (e.g. inflammatory cytokine) with an IC₅₀ of no more than 50 μM, 25 μM, 10 μM, 5 μM, 1 μM, 500 nM, 250 nM, 200 nM, 150 nM, 100 nM, 50 nM, 25 nM, 10 nM, 5 nM, 1 nM or no more than 0.1 nM. In some embodiments, the composition described herein inhibits release of a cytokine (e.g. inflammatory cytokine) with an IC₅₀ of about 1-100 PM, 0.5-50 PM, 1-10 μM, 1-100 nM, 0.1-50 nM, 50-500 nM, 10-100 nM, 0.1-100 nM, 100-500 nM, or 0.1-10 nM.

### Pharmaceutical Composition/Formulation.

A pharmaceutical composition can be a combination of any pharmaceutical compounds described herein (e.g., cannabinoids or extracts of cannabis plant material) with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, rectal, aerosol, parenteral, ophthalmic, pulmonary, transdermal, vaginal, otic, nasal, and topical administration. In some instances, a pharmaceutical composition comprises a cannabinoid and at least one excipient.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, and/or lubricants such as talc or magnesium stearate and, stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parental injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved and/or dispersed in a polymer or an adhesive. Such patches can be constructed for continuous, pulsatile, or on demand delivery of pharmaceutical compounds. Transdermal delivery can be accomplished by means of iontophoretic patches and the like. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin.

For administration by inhalation, the active compounds can be in a form as an aerosol, a mist, or a powder. Pharmaceutical compositions are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting wax such as a mixture of fatty acid glycerides or cocoa butter can be used.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising the compounds described herein can be manufactured, for example, by mixing, dissolving, granulating, dragee-making, levigating, emulsifying, encapsulating, entrapping, or compression processes.

The pharmaceutical compositions can include at least one pharmaceutically acceptable carrier, diluent, or excipient and compounds described herein as free-base or pharmaceutically-acceptable salt form. The methods and pharmaceutical compositions described herein include the use of crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents, and any combination thereof.

A composition can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, granular masses, and the like.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Effective dosages may also be determined from the blood or plasma concentration of drug. In some embodiments, the effective dosage for the acidic cannabinoid may be about 0.1 ng/mL to about 1000 ng/mL. In other instances, the effective dosage for the acidic cannabinoid may be about 0.5 ng/mL to about 1000 ng/mL. In still other instances, the effective dosage for the acidic cannabinoid may be about 1 ng/mL to about 900 ng/mL. In yet other instances, the effective dosage for the acidic cannabinoid may be about 5 ng/mL to about 700 ng/mL. In some instances, the effective dosage for the acidic cannabinoid may be about 10 ng/mL to about 500 ng/mL. In other instances, the effective dosage for the acidic cannabinoid may be about 15 ng/mL to about 400 ng/mL. In yet other instances, the effective dosage for the acidic cannabinoid may be about 20 ng/mL to about 300 ng/mL. In still other instances, the effective dosage for the acidic cannabinoid may be about 25 ng/mL to about 200 ng/mL. In some embodiments, the effective dosage for the acidic cannabinoid may be about 50 ng/mL to about 100 ng/mL.

In other instances, the effective dosage for the acidic cannabinoid may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the acidic cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In some embodiments, the effective dosage for the non-acidic cannabinoid may be about 0.1 ng/mL to about 1000 ng/mL. In other instances, the effective dosage for the non-acidic cannabinoid may be about 0.5 ng/mL to about 1000 ng/mL. In still other instances, the effective dosage for the non-acidic cannabinoid may be about 1 ng/mL to about 900 ng/mL. In yet other instances, the effective dosage for the non-acidic cannabinoid may be about 5 ng/mL to about 700 ng/mL. In some instances, the effective dosage for the non-acidic cannabinoid may be about 10 ng/mL to about 500 ng/mL. In other instances, the effective dosage for the non-acidic cannabinoid may be about 15 ng/mL to about 400 ng/mL. In yet other instances, the effective dosage for the non-acidic cannabinoid may be about 20 ng/mL to about 300 ng/mL. In still other instances, the effective dosage for the non-acidic cannabinoid may be about 25 ng/mL to about 200 ng/mL. In some embodiments, the effective dosage for the non-acidic cannabinoid may be about 50 ng/mL to about 100 ng/mL.

In other instances, the effective dosage for the non-acidic cannabinoid may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the non-acidic cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In still other instances, the effective dosage for acidic cannabinoid for the treatment of an individual in need thereof, may be about 0.1 mg/kg to about 50 mg/kg body weight. In other instances, the effective dosage for acidic cannabinoid may be about 0.01 mg/kg to about 500 mg/kg. In still other instances, the effective dosage for acidic cannabinoid may be about 0.1 mg/kg to about 500 mg/kg. In yet other instances, the effective dosage for acidic cannabinoid may be about 0.5 mg/kg to about 250 mg/kg. In some instances, the effective dosage for acidic cannabinoid may be about 0.5 mg/kg to about 100 mg/kg. In other instances, the effective dosage for acidic cannabinoid may be about 1 mg/kg to about 50 mg/kg. In yet other instances, the effective dosage for acidic cannabinoid may be about 2.5 mg/kg to about 50 mg/kg. In still other instances, the effective dosage for acidic cannabinoid may be about 5 mg/kg to about 40 mg/kg. In some embodiments, the effective dosage for acidic cannabinoid may be about 1 mg/kg to about 25 mg/kg.

In other instances, the effective dosage for acidic cannabinoid may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for acidic cannabinoid may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

In some embodiments, the effective dosage for a second cannabinoid in combination with acidic cannabinoid for the treatment of an individual in need thereof may be about 0.01 mg/kg to about 500 mg/kg. In other instances, the effective dosage for a second cannabinoid. In still other instances, the effective dosage for a second cannabinoid may be about 0.1 mg/kg to about 500 mg/kg. In yet other instances, the effective dosage for a second cannabinoid may be about 0.5 mg/kg to about 250 mg/kg. In some instances, the effective dosage for a second cannabinoid may be about 0.5 mg/kg to about 100 mg/kg. In other instances, the effective dosage for a second cannabinoid may be about 1 mg/kg to about 50 mg/kg. In yet other instances, the effective dosage for a second cannabinoid may be about 2.5 mg/kg to about 50 mg/kg. In still other instances, the effective dosage for a second cannabinoid may be about 5 mg/kg to about 40 mg/kg. In some embodiments, the effective dosage for a second cannabinoid may be about 1 mg/kg to about 25 mg/kg.

In other instances, the effective dosage for a second cannabinoid in combination with acidic cannabinoid for the treatment of an individual in need thereof may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for a second cannabinoid in combination with acidic cannabinoid may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Methods of Administration and Treatment Methods.

Pharmaceutical compositions containing compounds described herein (e.g., cannabinoids of Table 1) can be administered for prophylactic and/or therapeutic treatments. In some embodiments, compositions described herein are used to treat inflammatory diseases. In some embodiments, compositions described herein are used to treat pain (acute or chronic). In some embodiments, the inflammatory pain relates to pain from skin, joints or GI tract disease or disorders. In therapeutic applications, the compositions can be administered to a subject already suffering from a disease or condition, in an amount sufficient to cure or at least partially arrest the symptoms of the disease or condition, or to cure, heal, improve, or ameliorate the condition. Compounds can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

### Treatment of Pain

Compositions described herein may be used for the treatment of pain. In some embodiments, pain is described by duration, such as acute or chronic pain. In some instances, acute pain is relatively short term, caused by a specific stimulus such as surgery, dental work, burns/lacerations, childbirth/labor, or broken bones. In some embodiments, chronic pain is defined as pain lasting at least a week, two weeks, a month, two months, three months, six months, nine months, a year, two years, or more than 5 years. In some embodiments, chronic pain is defined as pain lasting at least six months. In some embodiments chronic pain is manifested or caused by headaches, arthritis, cancer, nerve pain, back pain, or fibromyalgia. In some embodiments, chronic or acute pain is nociceptive, neurogenic, or psychogenic pain. In some embodiments, pain is described based on the underlying cause of the pain (e.g., disease, disorder, or trauma). In some embodiments, pain is nociceptive, neurogenic, or psychogenic pain. In some embodiments, pain includes but is not limited to chronic pain, acute, nociceptive, breakthrough, soft tissue, visceral, somatic, phantom, cancer, inflammatory, or neuropathic pain. In some embodiments, pain is described relative to the area afflicted, such as head, skin, organs, muscles, tendons, spine, bone, or other part of the body.

Compositions described herein may be used to treat nociceptive pain. In some embodiments nociceptive pain includes but is not limited to radicular pain, somatic pain, or visceral pain. In some embodiments, radicular pain is caused by a radiculopathy, such as cervical, thoracic, or lumbar radiculopathy. In some embodiments, somatic pain is manifested by muscle pain, bone pain, skin pain, or headaches. In some embodiments, somatic pain is superficial (e.g., skin, mucus, and mucus membranes). In some embodiments, somatic pain is deep (tendons, joints, bones, muscles). In some embodiments, visceral pain is caused by inflammation. In some embodiments, somatogenic pain is muscular or skeletal (e.g., osteoarthritis, lumbosacral back pain, posttraumatic, myofascial), visceral (e.g., pancreatitis, ulcer, irritable bowel), ischemic (i.e., arteriosclerosis obliterans), or related to the progression of cancer (e.g., malignant or non-malignant).

Compositions described herein may be used to treat neurogenic pain. In some embodiments, neurogenic pain comprises neuropathic pain, central pain, or deafferentation pain. In some embodiments, neuropathic pain is caused by nerve damage or disease. In some embodiments, neuropathic pain comprises pain related to carpal tunnel syndrome, diabetic neuropathy, thalamic stroke and/or spinal cord injury. In some embodiments, central pain is caused by lesions of the central nervous system (e.g., thalamic pain). In some embodiments, deafferentation pain is caused by loss or interruption of sensory nerve fiber transmissions. In some embodiments, neurogenic pain is caused by posttraumatic and postoperative neuralgia. In some embodiments, neurogenic pain is caused by neuropathies (such as toxicity, or diabetes), causalgia, nerve entrapment, facial neuralgia, perineal neuralgia, postamputation, thalamic, or reflex sympathetic dystrophy.

Compositions described herein may be used to treat psychogenic pain. In some embodiments psychogenic pain results from psychological causes, such as mental, emotional, or behavioral factors. In some instances, psychogenic pain is manifested by headache, back pain, or stomach pain. In some instances, psychogenic pain is diagnosed by eliminating all other causes of pain.

Compositions described herein may be used to treat pain caused by specific disease, condition, disorder, or origin of pain. In some embodiments, compositions described herein are used to treat cancer pain (including metastatic or non-metastatic cancer), inflammatory disease pain, neuropathic pain, postoperative pain, iatrogenic pain (e.g., pain following invasive procedures or high dose radiation therapy, e.g., involving scar tissue formation resulting in a debilitating compromise of freedom of motion and substantial pain), complex regional pain syndromes, failed-back pain (e.g., acute or chronic back pain), soft tissue pain, joints and bone pain, central pain, injury (e.g., debilitating injuries, e.g., paraplegia, quadriplegia, etc., as well as non-debilitating injury (e.g., to back, neck, spine, joints, legs, arms, hands, feet, etc.)), arthritic pain (e.g., rheumatoid arthritis, osteoarthritis, arthritic symptoms of unknown etiology, etc.), hereditary disease (e.g., sickle cell anemia), infectious disease and resulting syndromes (e.g., Lyme disease, AIDS, etc.), headaches (e.g., migraine), causalgia, hyperesthesia, sympathetic dystrophy, phantom limb syndrome, denervation, and the like. In some embodiments, compositions described herein are used to treat pain associated with specific areas of the body, such as the musculoskeletal system, visceral organs, head, bones, tendons, skin, nervous system, or other area of the body.

### Treatment of Inflammatory Diseases

Compositions described herein may be used for the treatment or prevention of inflammatory diseases. In some embodiments, inflammatory diseases comprise diseases involving chronic inflammation. In some embodiments, such diseases include asthma, chronic peptic ulcer, tuberculosis, rheumatoid arthritis, periodontitis, ulcerative colitis, Crohn's disease, sinusitis, and active hepatitis. In some embodiments, an inflammatory disease comprises autoimmune disease. In some embodiments, such diseases include fibrosis, including Chronic Kidney Disease (CKD), renal fibrosis and other fibrotic diseases. In some embodiments, an inflammatory disease includes but is not limited to Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic Kidney Disease (CKD); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosis; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgG4-related sclerosing disease; Immune thrombocytopenic purpura (ITP); Inclusion body myositis (IBM); Interstitial cystitis (IC); Interstitial fibrosis (IF); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Renal (kidney) fibrosis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Tubulointerstitial fibrosis; Transverse myelitis; Type 1 diabetes; Ulcerative colitis (UC); Undifferentiated connective tissue disease (UCTD); Uveitis; Vasculitis; Vitiligo; and Vogt-Koyanagi-Harada Disease.

### Treatment of Neurodegenerative Diseases

Compositions described herein may be used for the treatment or prevention of neurodegenerative diseases. In some embodiments; neurodegenerative diseases comprise Alpers' Disease; Batten Disease; Batten Disease; Cerebro-Oculo-Facio-Skeletal Syndrome (COFS); Corticobasal Degeneration; Gerstmann-Straussler-Scheinker Disease; Kuru; Leigh's Disease; Monomelic Amyotrophy; Multiple System Atrophy; Multiple System Atrophy with Orthostatic Hypotension (Shy-Drager Syndrome); Neurodegeneration with Brain Iron Accumulation; Opsoclonus Myoclonus; Prion; Progressive Multifocal Leukoencephalopathy; Striatonigral Degeneration; Transmissible Spongiform Encephalopathies (Prion Diseases); Batten Disease; Alpers-Huttenlocher syndrome; alpha-methylacyl-CoA racemase deficiency; Andermann syndrome; Arts syndrome; ataxia neuropathy spectrum; autosomal dominant cerebellar ataxia; deafness; and narcolepsy; autosomal recessive spastic ataxia of Charlevoix-Saguenay; beta-propeller protein-associated neurodegeneration; CLN1 disease; CLN10 disease; CLN2 disease; CLN3 disease; CLN4 disease; CLN6 disease; CLN7 disease; CLN8 disease; congenital insensitivity to pain with anhidrosis; familial encephalopathy with neuroserpin inclusion bodies; fatty acid hydroxylase-associated neurodegeneration; GM2-gangliosidosis; AB variant; hereditary sensory and autonomic neuropathy type IE; hereditary sensory and autonomic neuropathy type II; hereditary sensory and autonomic neuropathy type V; infantile neuroaxonal dystrophy; infantile-onset ascending hereditary spastic paralysis; juvenile primary lateral sclerosis; Marinesco-Sjögren syndrome; mitochondrial membrane protein-associated neurodegeneration; multiple system atrophy; neuromyelitis optica; pantothenate kinase-associated neurodegeneration; polycystic lipomembranous osteodysplasia with sclerosing leukoencephalopathy; progressive external ophthalmoplegia; riboflavin transporter deficiency neuronopathy; Sandhoff disease; and spastic paraplegia type 49.

### Treatment of Proliferative Diseases

Compositions described herein may be used for the treatment or prevention of proliferative diseases. In some embodiments, the proliferative disease or condition comprises cancer. In some embodiments, the proliferative disease or condition comprises fibrosis. In some instances, proliferative disease or condition comprises lung/pulmonary, kidney, liver, heart, mediastinal, retroperitoneal cavity, bone marrow, or skin fibrosis, or scleroderma or systemic sclerosis. In some cases, the cancer is a solid tumor. Exemplary solid tumors include, but are not limited to, bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, or prostate cancer. In some cases, the solid tumor is a metastatic cancer. In some cases, the solid tumor is a relapsed or refractory cancer. In some embodiments, the cancer is bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, or prostate cancer. In some cases, the solid tumor is a metastatic cancer. In some cases, the solid tumor is a relapsed or refractory cancer. In some embodiments, the cancer is metastatic cancer. In some instances, the metastatic cancer comprises a metastatic bladder cancer, metastatic bone cancer, metastatic brain cancer, metastatic breast cancer, metastatic colorectal cancer, metastatic esophageal cancer, metastatic eye cancer, metastatic head and neck cancer, metastatic kidney cancer, metastatic lung cancer, metastatic melanoma, metastatic ovarian cancer, metastatic pancreatic cancer, or metastatic prostate cancer. In some embodiments, the cancer is a relapsed or refractory cancer. In some instances, the relapsed or refractory cancer comprises a relapsed or refractory bladder cancer, relapsed or refractory bone cancer, relapsed or refractory brain cancer, relapsed or refractory breast cancer, relapsed or refractory colorectal cancer, relapsed or refractory esophageal cancer, relapsed or refractory eye cancer, relapsed or refractory head and neck cancer, relapsed or refractory kidney cancer, relapsed or refractory lung cancer, relapsed or refractory melanoma, relapsed or refractory ovarian cancer, relapsed or refractory pancreatic cancer, or relapsed or refractory prostate cancer. In some embodiments, the cancer is a treatment-naïve cancer. In such cases, the treatment-naïve cancer is a cancer that has not been treated by a therapy. In some cases, the treatment-naive cancer is a solid tumor, such as bladder cancer, a bone cancer, a brain cancer, a breast cancer, a colorectal cancer, an esophageal cancer, an eye cancer, a head and neck cancer, a kidney cancer, a lung cancer, a melanoma, an ovarian cancer, a pancreatic cancer, or a prostate cancer. In some embodiments, the cancer is a hematologic malignancy. In some instances, the hematologic malignancy comprises a leukemia, a lymphoma, or a myeloma. In some cases, the hematologic malignancy is a T-cell malignancy. In other cases, the hematological malignancy is a B-cell malignancy. Exemplary hematologic malignancies include, but are not limited to, chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

### Administration

Multiple therapeutic agents can be administered in any order or simultaneously. In some embodiments, a therapeutic agent comprises a composition described herein (e.g., comprising a cannabinoid of Table 1). If simultaneously, the multiple therapeutic agents can be provided in a single, unified form, or in multiple forms, for example, as multiple separate pills. The compounds can be packed together or separately, in a single package or in a plurality of packages. One or all of the therapeutic agents can be given in multiple doses. If not simultaneous, the timing between the multiple doses may vary to as much as about a month.

Compounds and compositions can be packaged as a kit. In some embodiments, a kit includes written instructions on the use of the compounds and compositions.

Compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to lessen a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A compound can be administered as soon as is practicable after the onset of a disease or condition is detected or suspected, and for a length of time necessary for the treatment of the disease, such as, for example, from about 1 month to about 3 months. The length of treatment can vary for each subject.

### Dosage

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative. In some embodiments the pharmaceutical formulation unit dosage form is packaged into a container selected from the group consisting of a tube, ajar, a vial, a bag, a tray, a drum, a bottle, a syringe, a vape cartridge, and a can.

A compound described herein (e.g., an acidic and/or non-acidic cannabinoid) is in some embodiments present in a composition in a range of from about 1 mg to about 2500 mg; 1 mg to about 2000 mg; from about 5 mg to about 1000 mg, from about 5 mg to about 1200 mg, from about 10 mg to about 1000 mg, from about 25 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein (e.g., an acidic and/or non-acidic cannabinoid) in some embodiments is present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg, about 2100 mg, about 2200 mg, about 2300 mg, about 2400 mg or about 2500.

Effective dosages may also be determined from the blood or plasma concentration of drug. In some embodiments, the effective dosage for the acidic cannabinoid may be about 0.1 ng/mL to about 1000 ng/mL. In other instances, the effective dosage for the acidic cannabinoid may be about 0.5 ng/mL to about 1000 ng/mL. In still other instances, the effective dosage for the acidic cannabinoid may be about 1 ng/mL to about 900 ng/mL. In yet other instances, the effective dosage for the acidic cannabinoid may be about 5 ng/mL to about 700 ng/mL. In some instances, the effective dosage for the acidic cannabinoid may be about 10 ng/mL to about 500 ng/mL. In other instances, the effective dosage for the acidic cannabinoid may be about 15 ng/mL to about 400 ng/mL. In yet other instances, the effective dosage for the acidic cannabinoid may be about 20 ng/mL to about 300 ng/mL. In still other instances, the effective dosage for the acidic cannabinoid may be about 25 ng/mL to about 200 ng/mL. In some embodiments, the effective dosage for the acidic cannabinoid may be about 50 ng/mL to about 100 ng/mL.

In other instances, the effective dosage for the acidic cannabinoid may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the acidic cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In some embodiments, the effective dosage for the non-acidic cannabinoid may be about 0.1 ng/mL to about 1000 ng/mL. In other instances, the effective dosage for the non-acidic cannabinoid may be about 0.5 ng/mL to about 1000 ng/mL. In still other instances, the effective dosage for the non-acidic cannabinoid may be about 1 ng/mL to about 900 ng/mL. In yet other instances, the effective dosage for the non-acidic cannabinoid may be about 5 ng/mL to about 700 ng/mL. In some instances, the effective dosage for the non-acidic cannabinoid may be about 10 ng/mL to about 500 ng/mL. In other instances, the effective dosage for the non-acidic cannabinoid may be about 15 ng/mL to about 400 ng/mL. In yet other instances, the effective dosage for the non-acidic cannabinoid may be about 20 ng/mL to about 300 ng/mL. In still other instances, the effective dosage for the non-acidic cannabinoid may be about 25 ng/mL to about 200 ng/mL. In some embodiments, the effective dosage for the non-acidic cannabinoid may be about 50 ng/mL to about 100 ng/mL.

In other instances, the effective dosage for the non-acidic cannabinoid may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the non-acidic cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

### EXAMPLES

### Example 1

### High-Throughput 96-Well Microfluorimetric Bioassays

Example 1 describes the mechanism and the identification of proteins underlying store-operated calcium entry (SOCE).

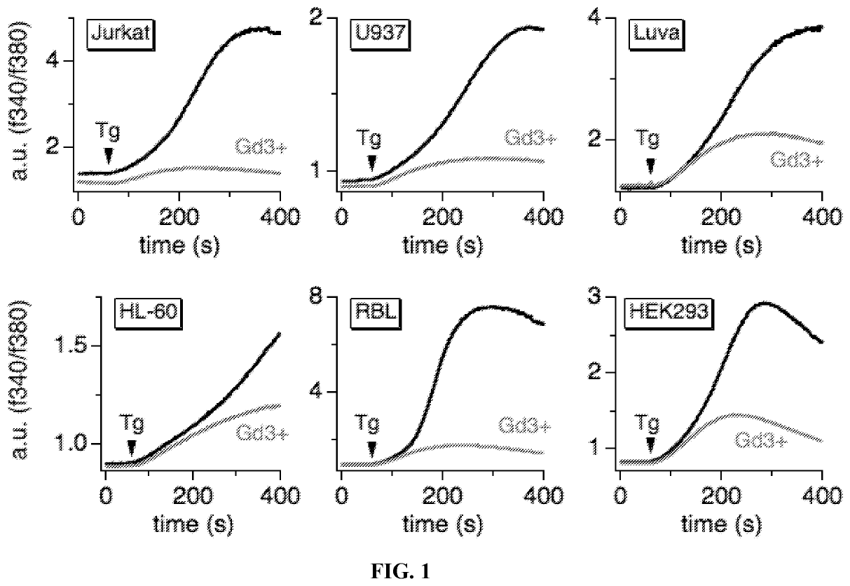

FIG. 1 shows examples of thapsigargin (Tg)-induced Ca²⁺ entry (SOCE) in a variety of immune cells (Jurkat T cells, U937 monocytes, Luva human mast cells, RBL-2H3 rat mast cells, and HL-60 neutrophils) and non-immune cells (HEK-293). A Fura-2 calcium flux assay was used to determine cytoplasmic Ca²⁺ levels. Briefly, cells were preloaded with Fura-2 AM, and fluorescence intensity in cells intensity was measured over time as a ratio of detected 510 nm fluorescent light emission intensity when excited by UV light at 340 nm and 380 nm wavelengths (f340/f380 ratio). Gadolinium (Gd³⁺) is known to block SOCE at a concentration of 1 μM and was used at 1 μM as a positive control in these experiments (gray line). As shown in FIG. 1, Tg treatment induced SOCE in all tested cell types as determined by f340/f380 ratio (black line), and Gd³⁺ inhibited SOCE in Tg-treated cells (gray line).

### Example 2

### Intact Population and Single-Cell Fluorescent Ca²⁺ Measurements with Fura-2-AM

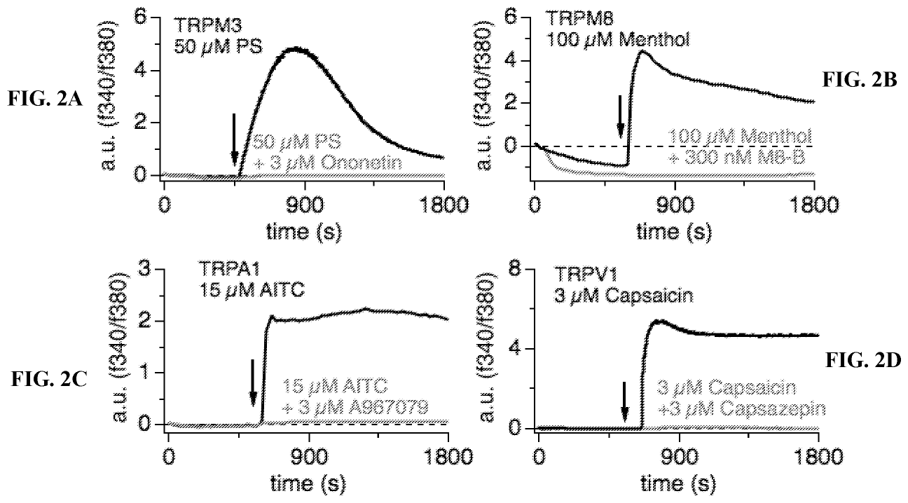

This example shows Fura-2 Ca²⁺ assays in 96- and 384-well high-throughput bioassay (HTS). HTS bioassays were developed to screen against four ion channels (TRPA1, TRPV1, TRPM3 and TRPM8) involved in pain sensation pathways (FIG. 2). These ion channels were overexpressed in tetracycline-inducible HEK293 cells, preloaded with Fura-2 AM as described in Example 1, and chemically stimulated to activate the overexpressed channels. HEK293 cells overexpressing TRPM3 were stimulated with 50 μM pregnenolone sulfate (PS) to activate TRPM3-mediated calcium mobilization (FIG. 2A, arrow; black line). HEK293 cells overexpressing TRPM3 channels were treated with 50 μM PS and 3 μM ononetin in control experiments to inhibit TRPM3-mediated calcium mobilization (FIG. 2A, arrow; gray line). HEK293 cells overexpressing TRPM8 were stimulated with 100 μM menthol to induce TRPM8-mediated calcium mobilization (FIG. 2B, arrow; black line). HEK293 cells overexpressing TRPM8 channels were treated with 100 μM menthol and 300 nM N-(2-aminoethyl)-N-[[3-methyoxy-4-(phenylmethoxy) phenyl]methyl]-2-thiophenecarboxamide, mono hydrochloride (M8-B) in control experiments to inhibit TRPM8-mediated calcium mobilization (FIG. 2B, arrow; gray line). HEK293 cells overexpressing TRPA1 were stimulated with 15 μM allyl isothiocyanate (AITC) to induce TRPA1-mediated calcium mobilization (FIG. 2C, arrow; black line). HEK293 cells overexpressing TRPA1 channels were treated with 15 μM AITC and 3 μM Δ967079 in control experiments to inhibit TRPA1-mediated calcium mobilization (FIG. 2C, arrow; gray line). HEK293 cells overexpressing TRPV1 were stimulated with 3 μM capsaicin to induce TRPV1-mediated calcium mobilization (FIG. 2D, arrow; black line). HEK293 cells overexpressing TRPV1 channels were treated with 3 μM capsaicin and 3 μM capsazepin in control experiments to inhibit TRPV1-mediated calcium mobilization (FIG. 2D, arrow; gray line).

FIG. 3 shows experimental data assessing agonist-induced Ca²⁺ oscillations, which can be a prerequisite for driving inflammatory cytokine release, in three individual human T lymphocytes (Jurkat cell line) using high-magnification Fura-2 fluorescence microscopy and digital image acquisition of single cells. Cytoplasmic calcium concentration oscillations were evoked by applying agonist phytohemagglutinin (PHA; 20 μg/ml), as shown in FIG. 3.

### Example 3

### Whole-Cell Patch Clamp Electrophysiology

This example shows experimental interrogation of calcium release mechanism in HEK293 immune cells using whole-cell patch clamping.

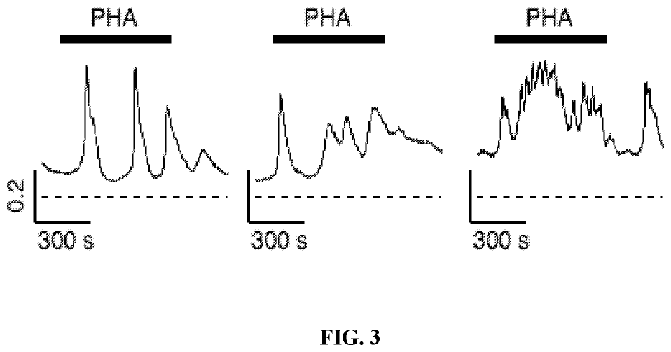

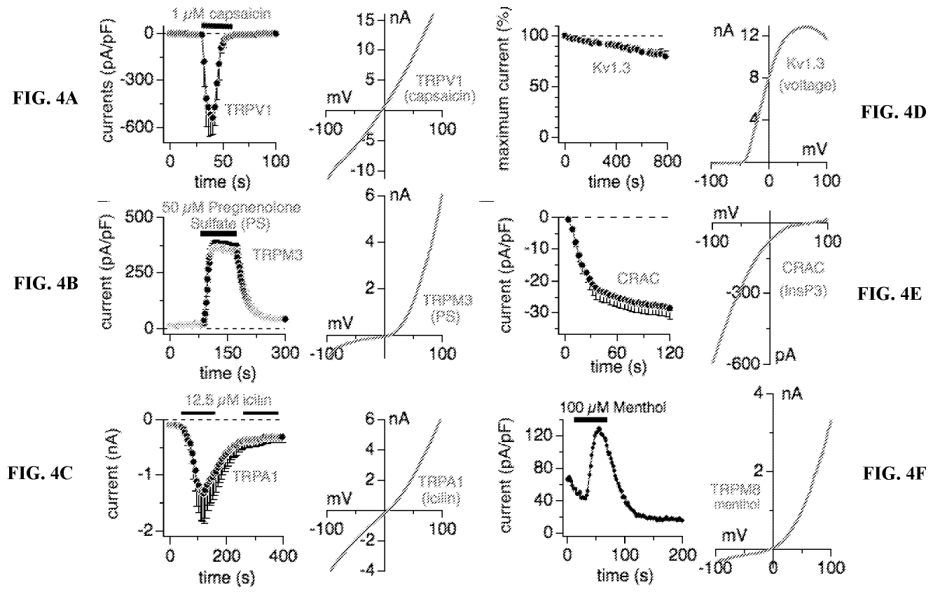

Whole-cell patch clamping was used to activate TRPV1 (FIG. 4A), TRPM3 (FIG. 4B), TRPA1 (FIG. 4C), Kv1.3 (FIG. 4D), I_{CRAC} (FIG. 4E) and TRPM8 (FIG. 4F) ion channels overexpressed in HEK293 cells. FIGS. 4A-4C and 4F: Left panels in each figure are averaged current development before, during and after agonist application (n=3-5, S.E.M.). Right panels are representative current-voltage traces extracted at the time of maximal current activation. Agonists used for assessing ion channel activity for TRPV1, TRPM3, TRPA1, Kv1.3, I_{CRAC}, and TRPM8 were 1 μM capsaicin, 50 μM pregnenolone sulfate (PS), 12.5 μM icilin, membrane depolarization, 50 μM inositol 1,4,5-trisphosphate (IP₃), and 100 μM menthol, respectively.

Voltage-gated potassium channel Kv1.3 was activated using a threshold voltage applied by the patch clamp pipette. I_{CRAC} channel activity was assessed using stimulation of inositol trisphosphate (IP₃) receptors with inositol trisphosphate. The left panels of FIGS. 4D and 4E show averaged current development of Kv1.3 currents by voltage activation (FIG. 4D) and CRAC currents (I_{CR}AC) by internal perfusion with 50 μM inositol 1,4,5-trisphosphate (IP₃) (FIG. 4E). Right panels are representative current-voltage traces extracted at the time of maximal current activation.

### Example 4

### Cytokine Release Assay

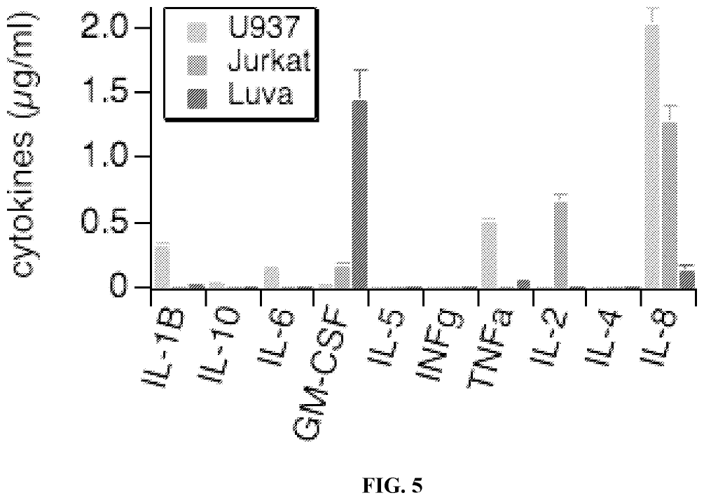

High-throughput multi-analyte bead-based immunoassay (Luminex® technology) combines a flow cytometer, fluorescently dyed microspheres (beads), lasers and digital signal processing to efficiently enable the detection and quantification of up to 100 targets within a single sample. Cytokine release in the immune cells of interest are shown in FIG. 5. The cytokine human 10-plex kit was used to simultaneously analyze a panel of the 10 most common pro-inflammatory cytokines (GM-CSF, IFNγ, IL-1β, IL-2, IL-4, IL-5, IL-6, IL-8, IL-10, TNF-α). In the assay, U937 cells, Jurkat cells, or Luva cells were seeded at a density of 2 million cells/ml and stimulated for 24 h. Supernatants were analyzed for cytokine content. Jurkat cells were stimulated with 50 ng/ml phorbol 12-myristate 13-acetate (PMA) and 1 μM ionomycin, U937 with 10 ng/ml PMA and 20 μg/ml lipopolysaccharide (LPS), Luva by 6 μM ionomycin. Results show that the respective agonists induce cytokine production in U937, Jurkat, and Luva cell lines.

### Example 5

### Analytical Chemistry: Extraction and Characterization of Phytochemicals

Plant material (Chemovar S04 obtained from NIDA) was subjected to a two-step extraction protocol using supercritical carbon dioxide to obtain terpene-rich (P≤1500 psi, T≤45° C.) and terpene-deficient (1500<P≤3500 psi, 45° C.<T≤60° C.) extracts. HPLC-DAD-MS analyses (HPLC-DAD is HPLC diode array detector analysis) of the extracts and commercial standards were used to identify known components (FIG. 6). FIG. 6 shows HPLC-UV (210 nm) traces of the terpene-deficient (TerpDefExt) and terpene-rich (TerpRichExt) extracts of the Cannabis plant material (NIDA Chemovar S04) and mixtures of commercial standards of terpenes and cannabinoids. Terpenes, flavonoids and lignans standards were obtained for use in these experiments. TerpMixA (terpene standards) comprised linalool (peak 2), β-myrcene (peak 13), terpinolene (peak 14), limonene (peak 18), α-pinene (peak 22). TerpMixB (terpene standards) comprised terpineol (peak 1), caryophyllene oxide (peak 8), ocimene (peak 12), 7-terpinene (peak 15), β-pinene (peak 19), A-carene (peak 21). TerpMixC (terpene standards): fenchol (no UV), camphene (peak 16), α-phellandrene (peak 17), α-humulene (peak 27), p-caryophyllene (peak 28). CB Std. (cannabinoid standards) comprised CBDVA (peak 3), CBND (peak 4), CBDV (peak 5), CBDA (peak 6), CBGA (peak 7), CBG (peak 9), CBD (peak 10), THCV (peak 11), CBN (peak 20), Δ⁹-THC (peak 23), Δ⁸-THC (peak 24), THCA (peak 25), CBC (peak 26). HPLC-DAD analysis showed that terpene-deficient extracts comprised CBDA, CBD and/or THCV, Δ⁹-THC, and THCA and/or CBC. Terpene-rich extracts were found to comprise β-myrcene (13), α-humulene (27), and β-caryophyllene (28).

FIG. 6. HPLC-UV (210 nm) traces of the terpene-deficient (TerpDefExt) and terpene-rich (TerpRichExt) extracts of the Cannabis plant material (NIDA Chemovar S04) and mixtures of commercial standards of terpenes and cannabinoids. TerpMixA (terpene standards): linalool (2), β-myrcene (13), terpinolene (14), limonene (18), α-pinene (22). TerpMixB (terpene standards): terpineol (1), caryophyllene oxide (8), ocimene (12), γ-terpinene (15), β-pinene (19), A-carene (21). TerpMixC (terpene standards): fenchol (no UV), camphene (16), α-phellandrene (17), α-humulene (27), 0-caryophyllene (28). CB Std. (Cannabinoid standards): CBDVA (3), CBND (4), CBDV (5), CBDA (6), CBGA (7), CBG (9), CBD (10), THCV (11), CBN (20), Δ⁹-THC (23), Δ⁸-THC (24), THCA (25), CBC (26).

Cannabis Phytochemicals and Whole-Cannabis Plant Materials

Whole-plant cannabis extracts and whole-plant dried cannabis samples/specimens (chemovars) with various THC/CBD ratios was obtained, as well individual cannabinoids. Terpenes, flavonoids and lignans standards were obtained (Tables 2 and 3). Samples S01-S011) were tested in human cells for SOCE inhibition (Table 4).

Table 2 shows percentages of various cannabinoids in NIDA raw plant material samples.

| | NIDA Raw Plant Material | | | | | | | | | | |
|---|---|---|---|---|---|---|---|---|---|---|---|
| weight % in | | | | | | | | | | | |
| extract | S01 | S02 | S03 | S04 | S05 | S06 | S07 | S08 | S09 | S10 | S11 |
| CBDVA | 0.0 | 0.0 | 0.5 | 0.2 | 0.3 | 0.5 | 0.2 | 0.1 | 0.1 | 0.0 | 0.2 |
| CBND | 0.0 | 0.0 | 0.0 | — | — | 0.0 | — | — | — | — | — |
| CBGVA | — | — | — | — | — | — | — | — | — | — | — |
| CBDV | 0.2 | 0.1 | 0.1 | 0.1 | 0.1 | 0.1 | 0.2 | 0.1 | 0.1 | 0.3 | 0.1 |
| CBDA | 0.1 | 0.1 | 24.0 | 25.2 | 37.5 | 41.9 | 28.1 | 22.5 | 18.5 | 0.1 | 20.5 |
| CBGA | 1.1 | 1.3 | 0.5 | 1.4 | 2.0 | 1.3 | 2.0 | 1.0 | 1.0 | 0.3 | 0.9 |
| CBG | 0.3 | 0.3 | 0.1 | 0.3 | 0.3 | 0.2 | 0.3 | 0.3 | 0.3 | 0.2 | 0.2 |
| CBD | 0.1 | 0.1 | 3.8 | 2.8 | 2.9 | 3.1 | 1.9 | 1.9 | 2.0 | 0.1 | 2.2 |
| THCV | 0.1 | 0.1 | 0.0 | 0.1 | 0.1 | 0.1 | 0.1 | 0.1 | 0.1 | 0.1 | 0.1 |
| CBCV | — | — | — | — | — | — | — | — | — | — | — |
| THCVA | 0.2 | 0.3 | 0.0 | 0.2 | 0.3 | 0.0 | 0.3 | 0.1 | 0.1 | 0.1 | 0.1 |
| CBN | 0.8 | 0.7 | 0.1 | 0.3 | 0.2 | 0.0 | 0.3 | 0.2 | 0.2 | 0.5 | 0.1 |
| CBNA | 1.6 | 1.6 | 0.1 | 0.7 | 0.6 | 0.1 | 0.9 | 0.7 | 0.6 | 1.2 | 0.3 |
| Δ9-THC | 3.5 | 3.7 | 0.3 | 2.3 | 2.2 | 0.2 | 2.6 | 1.5 | 1.6 | 1.0 | 0.8 |
| Δ8-THC | — | — | 0.0 | 0.0 | 0.0 | 0.0 | — | — | 0.0 | 0.0 | 0.0 |
| CBL | 0.2 | 0.3 | — | — | — | — | 0.3 | 0.2 | — | — | — |
| CBC | 0.2 | 0.1 | 0.2 | 0.2 | 0.2 | 0.1 | 0.2 | 0.1 | 0.1 | 0.1 | 0.1 |
| THCA | 30.9 | 35.8 | 1.0 | 12.3 | 19.3 | 1.2 | 24.4 | 11.5 | 9.4 | 8.3 | 4.8 |
| CBCA | 0.8 | 0.7 | 0.9 | 1.1 | 1.8 | 1.5 | 1.6 | 1.0 | 1.0 | 0.7 | 0.9 |
| CBLA | 0.1 | 0.1 | 0.1 | 0.1 | 0.1 | 0.1 | 0.1 | 0.1 | 0.1 | 0.1 | 0.1 |
| CBNM | — | — | — | — | — | — | — | — | — | — | — |

Table 3 shows percentages of various cannabinoids in extracts derived from NIDA samples and hemp.

| | | | | Hemp | | | | | | | | | | | |
|---|---|---|---|---|---|---|---|---|---|---|---|---|---|---|---|
| weight % | NIDA Extracts | | | | | | White | Cherry | Suver | | | Sour Space | Special | Hawaiian | Grape |
| in extract | CBD | THC1 | THC2 | Otto | Har | Boax | CBG | Wine | Haze | Lifter | Elektra | Candy | Sauce | Haze | Soda |
| CBDVA | 0.0 | — | — | 0.2 | 0.1 | 0.0 | 0.0 | 0.6 | 0.8 | 0.7 | 0.7 | 0.9 | 0.8 | 0.9 | 0.6 |
| CBND | 0.0 | — | 0.2 | 0.0 | — | 0.0 | — | — | 0.0 | 0.0 | 0.0 | — | 0.0 | 0.0 | 0.0 |
| CBGVA | — | — | — | — | — | — | — | — | — | — | — | — | — | — | — |
| CBDV | 0.9 | 0.3 | 0.3 | 0.4 | 0.1 | 0.0 | — | — | 0.3 | 0.1 | 0.1 | 0.1 | 0.3 | 0.2 | 0.0 |
| CBDA | 0.2 | 0.1 | 0.1 | 3.5 | 30.0 | 5.0 | 0.2 | 60.5 | 61.1 | 57.9 | 61.6 | 64.3 | 59.3 | 65.7 | 44.2 |
| CBGA | — | — | 0.4 | 0.3 | 1.0 | 0.0 | 60.1 | 0.9 | 1.3 | 1.6 | 0.9 | 1.1 | 2.8 | 1.3 | 0.9 |
| CBG | 1.5 | 0.5 | 0.3 | 0.5 | 0.2 | 0.0 | 2.3 | 0.2 | 0.2 | 0.3 | 0.3 | 0.3 | 0.3 | 0.2 | 0.2 |
| CBD | 50.0 | 1.6 | 1.4 | 6.0 | 5.0 | 0.7 | 0.1 | 9.9 | 4.4 | 5.8 | 5.4 | 9.1 | 3.4 | 3.7 | 4.3 |
| THCV | 0.1 | 0.8 | 0.5 | 0.8 | 0.0 | — | 0.2 | 0.0 | 0.0 | — | 0.0 | 0.0 | 0.0 | — | — |
| CBCV | 0.0 | 0.0 | 0.0 | 0.0 | — | — | — | — | — | — | 0.0 | 0.0 | — | — | — |
| THCVA | 0.0 | — | — | 0.2 | — | — | 0.1 | 0.1 | 0.0 | 0.1 | 0.0 | 0.0 | 0.1 | 0.1 | 0.0 |
| CBN | 0.2 | 1.3 | 2.4 | 0.3 | 0.0 | 0.1 | 0.0 | 0.0 | 0.0 | 0.0 | 0.0 | 0.0 | 0.0 | 0.0 | 0.0 |
| CBNA | — | 0.1 | 0.1 | 0.1 | 0.0 | — | 0.0 | 0.0 | 0.0 | — | 0.0 | 0.0 | 0.0 | 0.0 | 0.0 |
| Δ9-THC | 2.0 | 29.6 | 19.3 | 7.5 | 0.4 | 0.0 | 0.2 | 1.1 | 0.5 | 0.7 | 0.6 | 1.0 | 0.5 | 0.4 | 0.4 |
| Δ8-THC | 0.0 | 0.0 | 0.0 | — | 0.0 | 0.0 | 0.0 | 0.0 | 0.0 | 0.0 | 0.0 | 0.0 | 0.0 | 0.0 | 0.0 |
| CBL | 0.1 | 0.3 | 0.3 | 0.0 | 0.0 | — | 0.2 | — | — | 0.4 | — | — | 0.1 | — | — |
| CBC | 2.5 | 2.3 | 2.0 | 0.6 | 0.5 | 0.1 | 0.3 | 0.7 | 0.4 | 0.4 | 0.4 | 0.5 | 0.3 | 0.3 | 0.2 |
| THCA | 0.0 | 0.2 | 0.1 | 2.0 | 1.0 | 0.2 | 0.6 | 1.6 | 1.8 | 1.8 | 1.7 | 1.7 | 2.1 | 2.0 | 1.6 |
| CBCA | — | 0.3 | 0.2 | 0.3 | 2.8 | 0.7 | 1.4 | 2.5 | 3.5 | 2.5 | 2.7 | 2.2 | 3.2 | 2.9 | 2.0 |
| CBLA | — | 0.1 | 0.1 | 0.1 | 0.0 | 0.0 | — | 0.0 | 0.0 | 0.0 | 0.0 | — | 0.0 | 0.0 | 0.0 |
| CBNM | — | 0.1 | — | — | — | — | — | 0.1 | 0.1 | — | 0.1 | — | 0.0 | 0.1 | 0.0 |

Table 4 shows IC₅₀ values for various cannabinoids in NIDA raw plant material samples, extracts, and hemp in an SOCE inhibition experiment with various human cell lines.

| | IC50 (ug/mL) | Jurkat | U937 | Luva | RBL-2H3 | HEK293 |
|---|---|---|---|---|---|---|
| NIDA | S01 | 1.7 | 9.2 | 16 | 8.3 | 14 |
| Raw | S02 | 1.6 | 10 | 18 | 7.4 | 12 |
| Plant | S03 | 2.1 | 7.8 | 8.8 | 9.4 | 16 |
| Material | S04 | 1.9 | 9.2 | 9.2 | 7 | 10 |
| | S05 | 1.1 | 2.4 | 3.1 | 5.2 | 6 |
| | S06 | 1.5 | 5.8 | 7.4 | 5.9 | 8.8 |
| | S07 | 1.1 | 2 | 3.2 | 5.4 | 4.7 |
| | S08 | 1.9 | 9.1 | 10 | 8.2 | 10 |
| | S09 | 1.7 | 5.4 | 11 | 8.9 | 9.4 |
| | S10 | 5.9 | 22 | >50 | 22 | 18 |
| | S11 | 2.1 | 6.6 | 11 | 9.2 | 10 |
| NIDA | CBD | 5.1 | 24 | 12 | >50 | 33 |
| Extracts | THC1 | 9.8 | 37 | >50 | >50 | >50 |
| | THC2 | 9 | 35 | 42 | 44 | 39 |
| Hemp | Otto | 12 | 31 | 4.2 | >50 | >50 |
| (μg/ml) | Har | 4.2 | 6.6 | 1.2 | 10 | 19 |
| | Boax | 22 | 37 | >50 | >50 | >50 |

TABLE 5
illustrates the activity of various fractions in an SOCE
experiment with human cells.

| IC₅₀ | | | | | |
|---|---|---|---|---|---|
| ug/mL | Jurkat | U937 | Luva | RBL-2H3 | HEK-293 |
| CBD | 7.42 | >50 | >50 | 2.53 | >50 |
| CBDA | 4.07 | 5.83 | 5.17 | 2.4 | 16.13 |
| CBG | 2.36 | 33.52 | 33.63 | 3.29 | >50 |
| CBGA | 0.53 | 2.23 | 2.21 | 0.27 | 5.72 |
| Δ9-THC | >50 | >50 | >50 | 0.97 | >50 |
| THCA | 2.23 | 8.1 | 10.39 | 0.57 | 17.53 |
| CBDV | 6 | 17.49 | >50 | 0.17 | 30.18 |
| CBDVA | 11.29 | 10.64 | 8.45 | 11.32 | 23.52 |
| CBGV | 5.27 | 14.51 | 40.7 | 6.46 | 30.85 |
| CBGVA | 1.15 | 2.86 | 2.6 | 2.53 | 4.15 |
| CBL | >50 | >50 | >50 | >50 | >50 |
| CBLA | 9.29 | 28.32 | 12.37 | 20.85 | >50 |
| CBNA | 0.99 | 19.18 | 7.25 | >50 | 22.49 |
| CBCA | 1.13 | >50 | 29.13 | >50 | >50 |
| THCVA | 0.633 | 20.16 | 9.43 | 20.21 | 21.34 |
| CBCV | 10.6 | >50 | >50 | 36.06 | 49.11 |
| CBND | 6.7 | 24 | >50 | 19 | 32.8 |
| THCV | 10.73 | >50 | >50 | >50 | >50 |
| CBN | 9.09 | 45.3 | >50 | 33.4 | 42.6 |
| Δ8-THC | 14.18 | >50 | >50 | 39.7 | >50 |
| CBC | 13.06 | 40.8 | >50 | 26.8 | 31.6 |
| CBNM | >50 | >50 | >50 | >50 | >50 |

### Example 6

### Individual and Combinatorial Cannabinoid Inhibition of SOCE in HTS Bioassays

HTS Bioassay of Cannabinoids on SOCE.

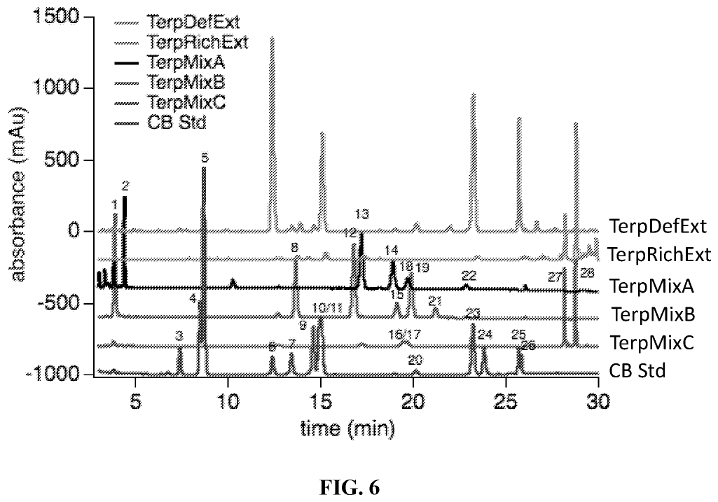

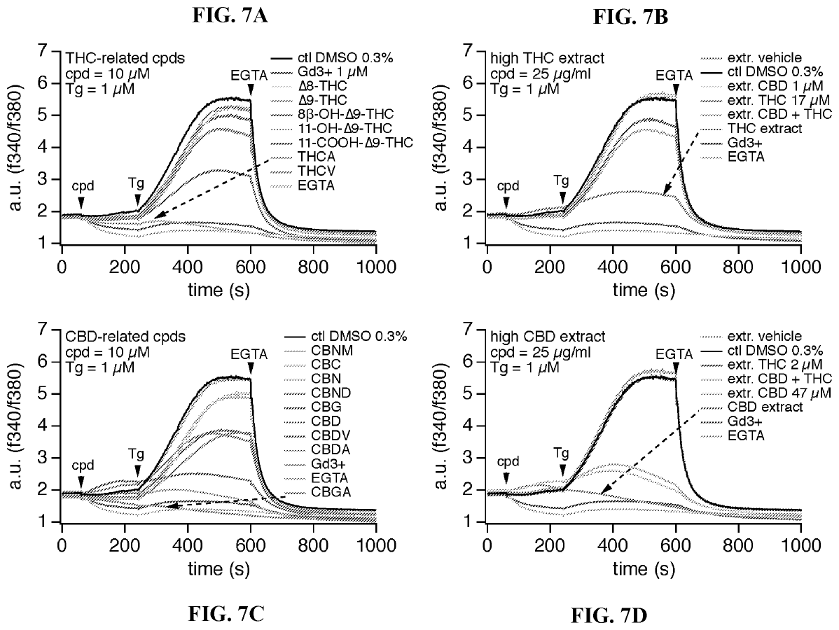

SOCE is the main Ca²⁺ entry mechanism and upstream signaling pathway in immune cell activation. Cannabis extracts and cannabinoids were screened against SOCE in Jurkat T cells (FIGS. 7A-D). SOCE was experimentally solicited in intact Jurkat cells by applying 1 μM thapsigargin (Tg). Prior to this, the cells were pre-loaded with the Ca²⁺ sensitive dye Fura-2-AM (2 μM) for 1 hour. After washing the excess dye, cells were seeded in the assay plate (96-well plate) in physiological Ringer's solution containing 1 mM Ca²⁺. Fura-2 fluorescence emitted at 500 nm was then measured at excitation wavelengths of 340 and 380 nm. Emitted fluorescence intensities were processed by ratio analysis to obtain free intracellular Ca²⁺ concentrations. After obtaining baseline levels for 60 s, cannabis extracts or individual components were added to the individual wells of the assay plate and the resulting changes in [Ca²⁺ ]ᵢ were continuously monitored. Applying these compounds first allowed a determination of whether or not these compounds had any calcium activation effects. At 240 s, thapsigargin was then applied to trigger the activation of SOCE. At the end of each assay, the calcium chelator, EGTA, was applied to confirm that the recorded signals are indeed a result of Ca²⁺ influx and to test for possible inhibition of the PMCA. The inhibitory effect of each cannabis compound or extract is then calculated by measuring the amplitude of SOCE in the presence of these compounds or extracts compared to the amplitude in untreated cells. All extracts were screened at 25 μg/ml, whereas the pure compounds were tested at 10 μM.

FIG. 7. Effect of cannabinoids on SOCE in Jurkat cells. Calcium signals are solicited in intact cells by applying 1 μM thapsigargin (Tg). Gadolinium (1 μM) is used here as a positive control (pos ctl) of SOCE inhibition. Seven THC derivatives (7A), one high-THC extract (7B), nine CBD derivatives (7C) and one high-CBD extract (7D) were screened. The compounds and extracts were tested at 10 μM and 25 μg/ml, respectively. All data are averages of 3 independent runs.

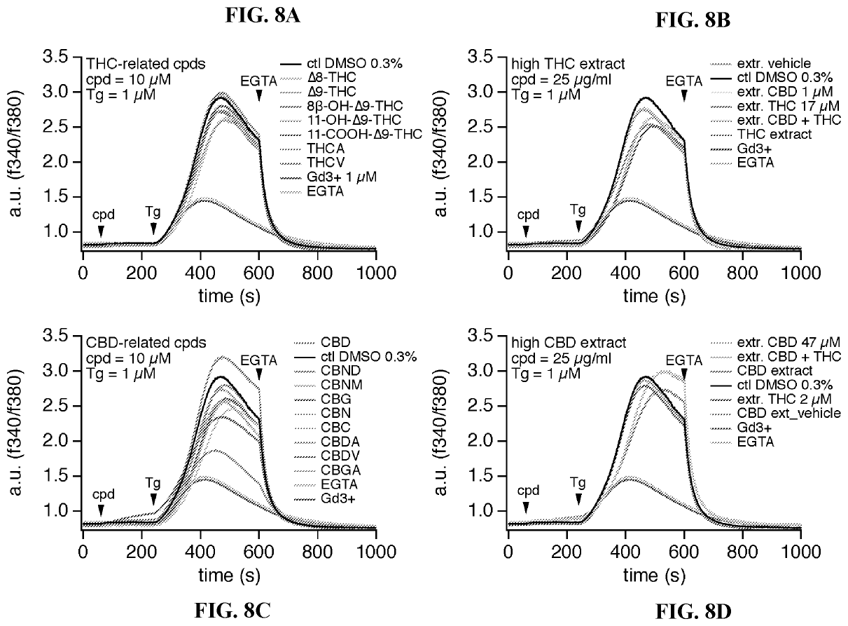

While CBD and THC show low to no effects at 10 μM, other minor cannabinoids were able to fully block the Ca²⁺ signals, with potency similar to that of gadolinium (a known SOCE inhibitor, used here as a positive control) (FIG. 7A, C). High-THC extract inhibited SOCE but its effect was not due to its THC or CBD contents (FIG. 7B). The high-CBD extract, on the other hand, showed full block of SOCE, an inhibition that is mostly, but not fully, due to its high CBD content of 47 μM (FIG. 7D). A screening of a non-immune cell line, HEK293 shown in FIGS. 8A-D, demonstrates that the inhibitory effects of the tested cannabinoids and extracts may be selective (e.g., 11-nor-COOH-Δ9-THC, THCV, CBDV, CBDA) or more potent (e.g., CBGA) in immune cells. Without being bound by theory, these effects may be reflective of the difference in the expression profile of various Ca²⁺ players involved in immune cells vs. the ones involved in fibroblast HEK293 cells.

FIG. 8. Effect of cannabinoids on SOCE in HEK293 cells. Calcium signals were solicited in intact cells by applying 1 μM thapsigargin (Tg). Gadolinium (1 μM) was used as a positive control (pos ctl) of SOCE inhibition. Seven THC derivatives were screened (8A), a high-THC extract (8B), nine CBD derivatives (8C) and a high-CBD extract (8D). The compounds and extracts were tested at 10 μM and 25 μg/ml, respectively. All data are averages of 3 independent runs.

### Example 7

### Concentration Response Effect of Cannabinoids

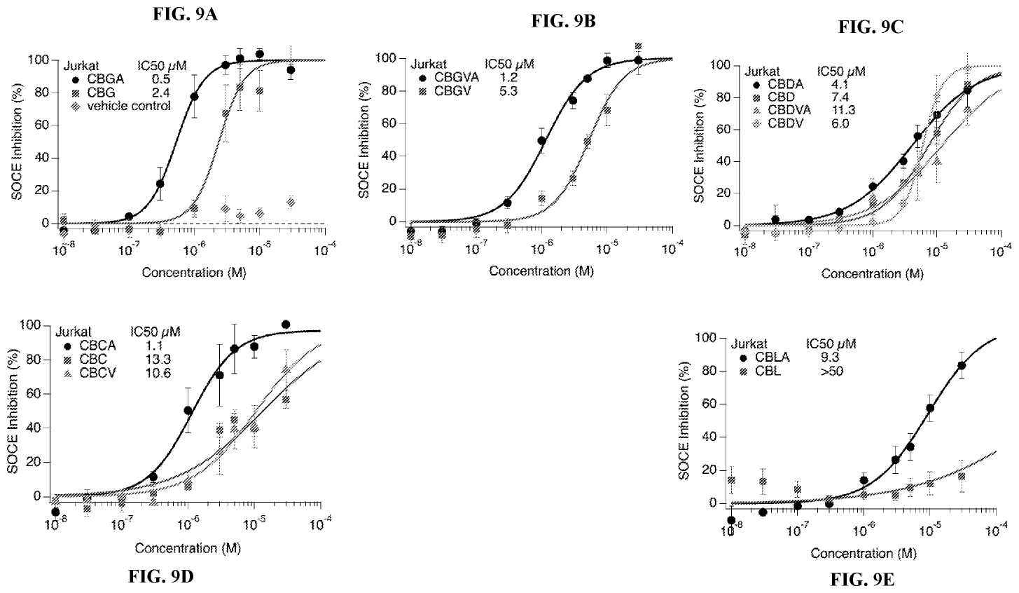

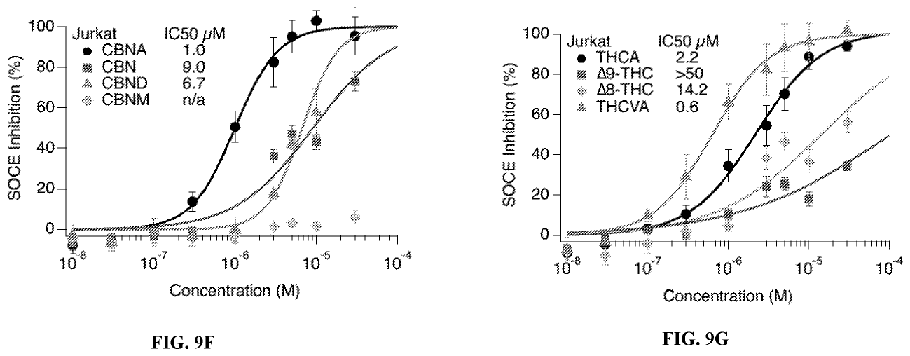

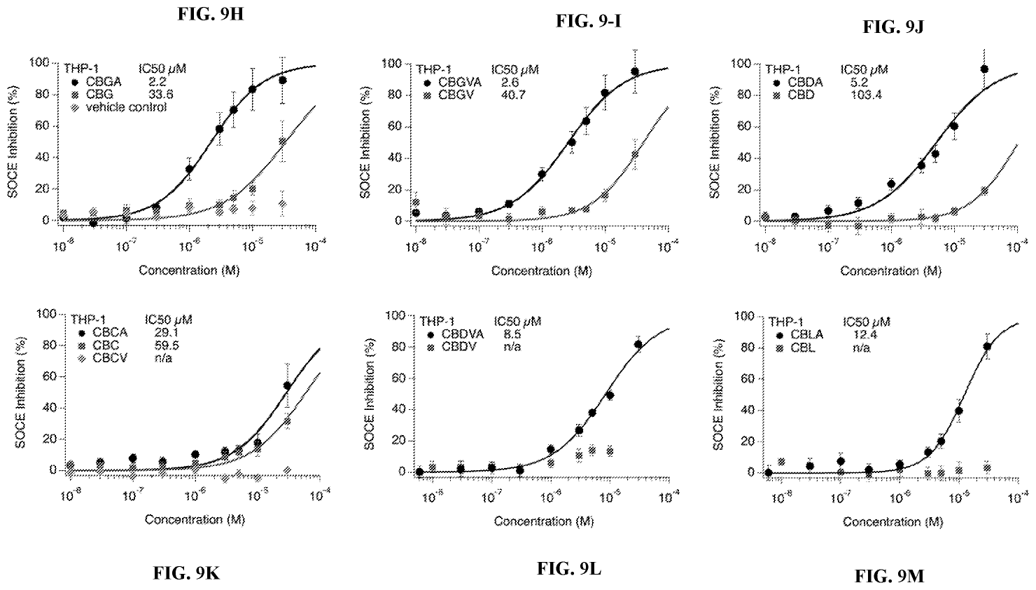

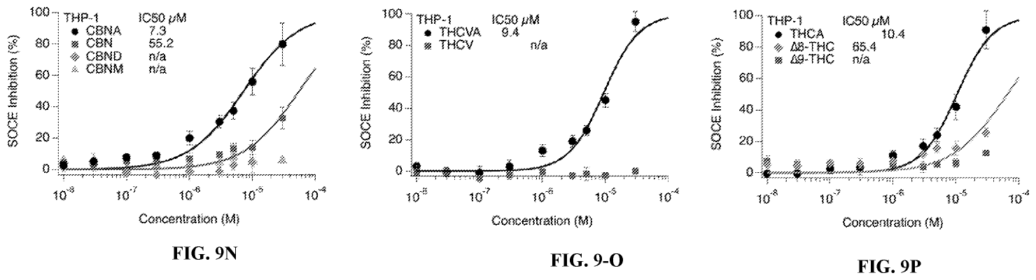

The minor cannabinoids that showed an inhibition rate of 40% or higher, particularly in Jurkat cells (FIGS. 7A-D), were then screened for their dose-response behavior along CBD and THC compounds. The dose response behavior was carried out in Jurkat cells. FIGS. 9A-P shows cannabinoids like CBD, CBG and THC have a higher IC₅₀ than their acidic variations.

FIGS. 9A-P. Dose-response behavior of cannabinoids on SOCE. Hit cannabinoids from the prescreening in FIGS. 9A-P were tested at 9 different doses from 0 to 30 μM. The effects on SOCE amplitude were plotted in a dose-dependent manner to determine the IC₅₀ for each tested compound.

### Example 8

### Combinatory Effects of Cannabinoids

This example shows analysis of combinatory effect of cannabinoids on store-operated calcium entry (SOCE) in human cells.

Part of the complexities of cannabis use, as well as the large variability in outcomes, are linked to a combinatory effect, which is also known as entourage effect or ensemble effect. The existence of various chemovars of cannabis as well as the multiple modes of use (e.g., edibles, vaporization, etc.) will affect the amounts and compositions of what is administered to the patients and, hence, the outcomes and degrees of efficacy for a given indication. In some cases, combinatory effect is beneficial. In some cases, combinatory effect is undesired. A prominent example of an undesired combinatory effect is in the use of THC for the treatment of glaucoma by lowering intraocular pressure. Without being bound by theory, the presence of CBD in a treatment comprising THC may antagonize THC effects, in some cases.

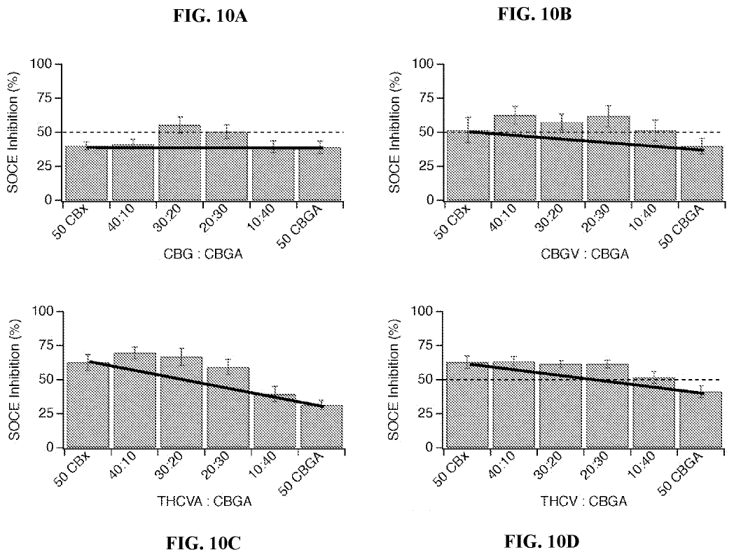

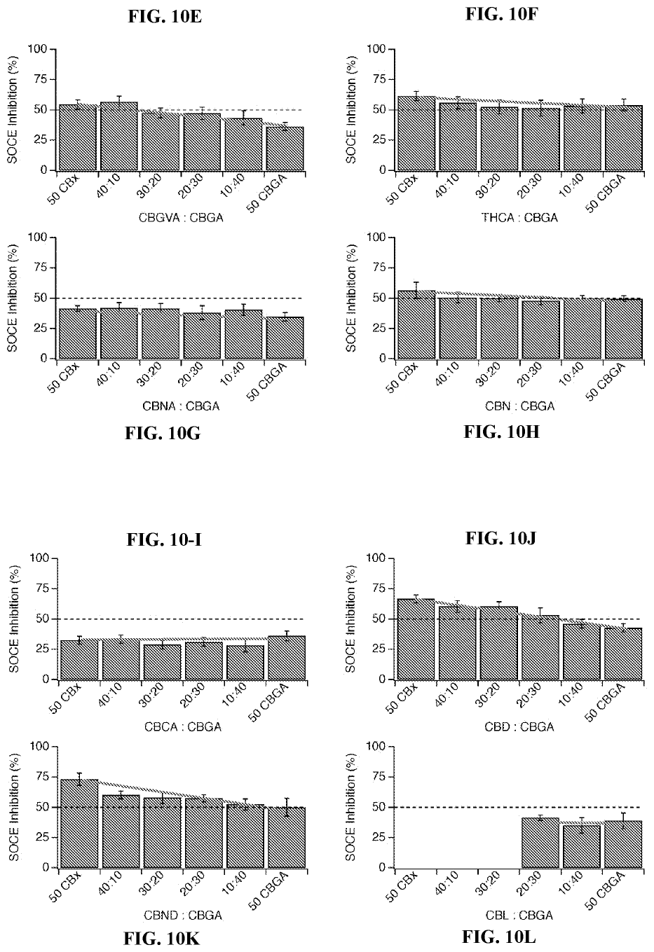

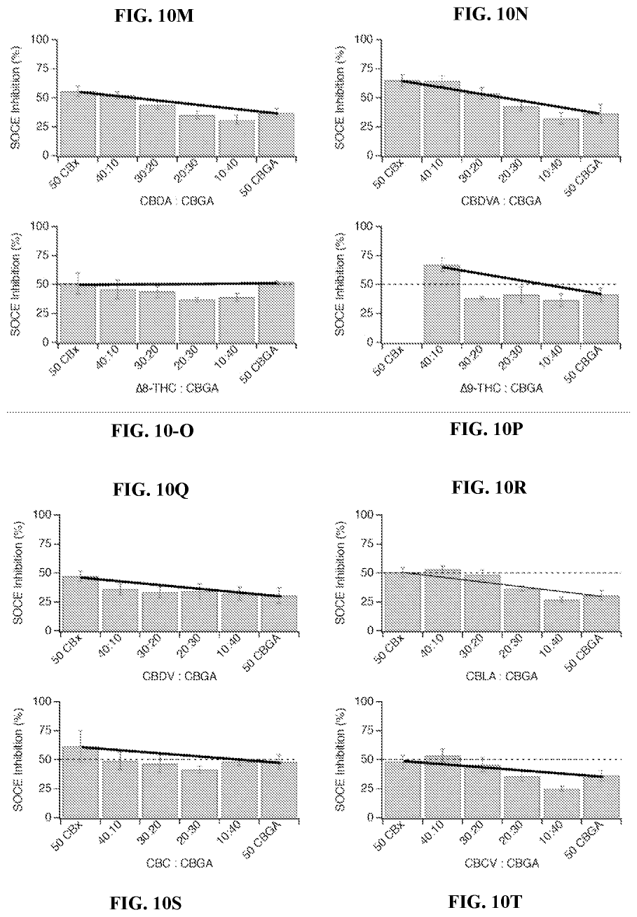

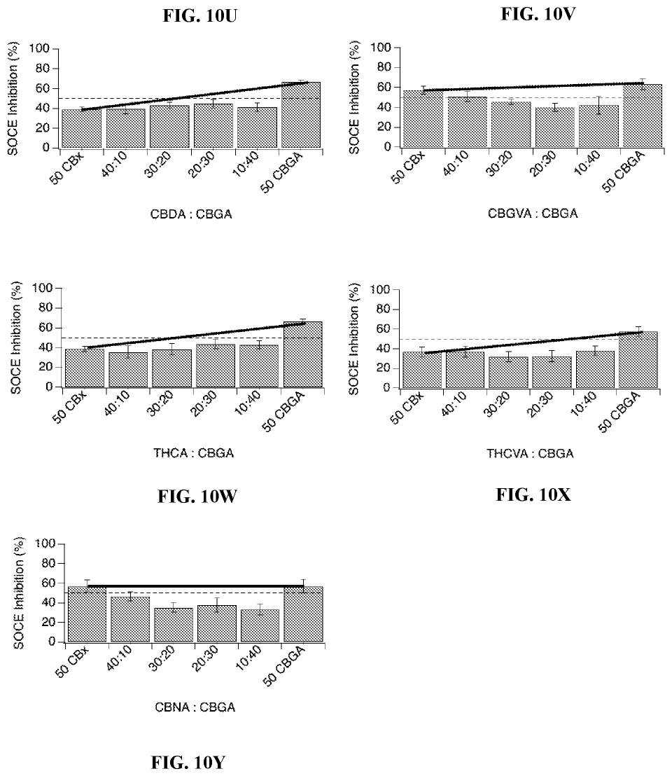

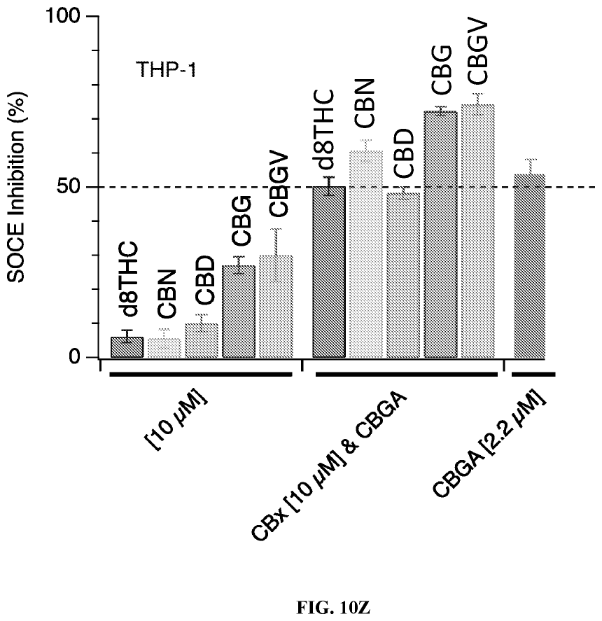

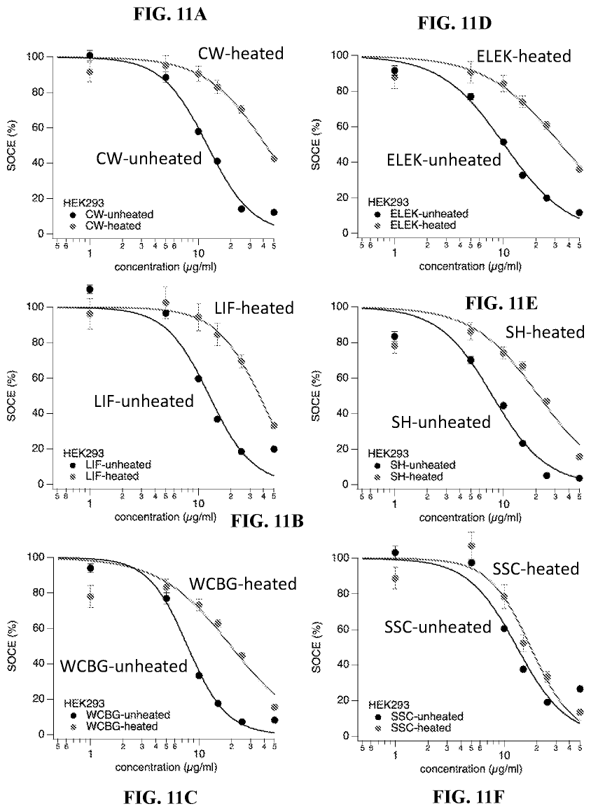

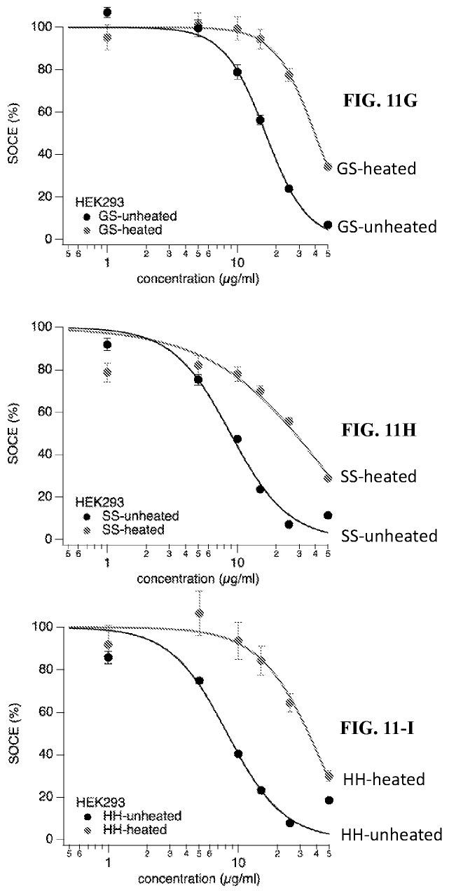

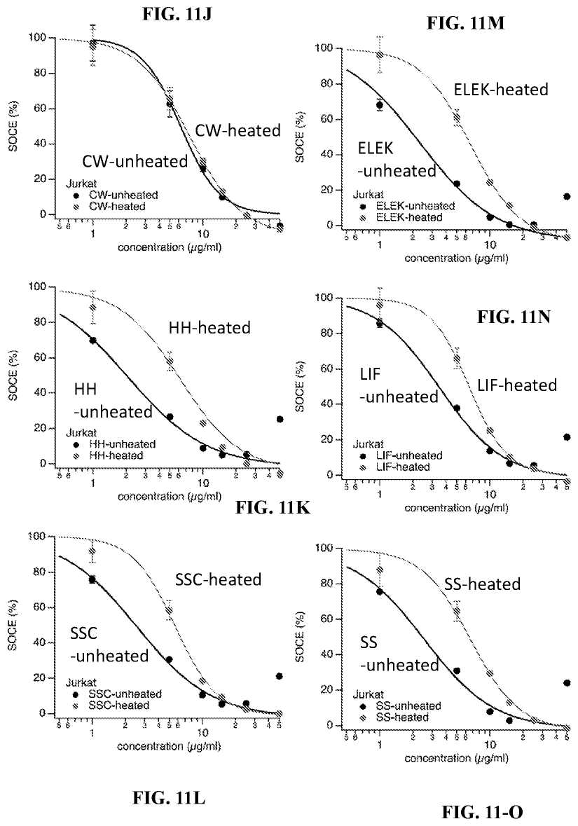

Combinatory effect studies using the most potent hit (e.g., candidate) from initial screening against Ca²⁺ signaling, namely CBGA (for example, as shown in Examples 6 and 7), in combination with other cannabinoids was conducted (FIGS. 10A-10BB). Bar graphs shown in FIGS. 10A-10Z and 10BB display SOCE amplitudes calculated from area under the curve (AUC) of the SOCE signals and normalized to SOCE in the presence of gadolinium. Combinatory effects were assessed using an isobolographic analysis approach, where CBGA was paired with another cannabinoid using concentrations of CBGA corresponding to 50%, 40%, 30%, 20%, 10%, and 0% inhibition (as derived from the IC₅₀ curves of FIGS. 9A-9P) and combined with concentrations of the paired cannabinoid, so that each combination would be expected to inhibit 50% of the Ca²⁺ signal (e.g., the CBGA concentration causing 50% inhibition was tested alone without the other cannabinoid, the CBGA concentration expected to block 40% of the signal was combined with the concentration of the other cannabinoid expected to inhibit 10%, the CBGA concentration expected to block 30% of the signal was combined with the concentration of the other cannabinoid expected to inhibit 20%, and so on). Simple additivity of cannabinoid pairs would be expected to inhibit the Ca²⁺ signal by 50%. All the data in these experiments represent an average of 3 independent runs and the values are graphed as mean values±SEM.

FIGS. 10A-10Y illustrate the inhibition of SOCE by paired cannabinoids comprising CBGA (e.g., via combinatory effect). FIGS. 10A-10T show inhibition of SOCE by individual or paired cannabinoids in Jurkat cells. FIGS. 10U-10Y show inhibition of SOCE by individual or paired cannabinoids in THP-1 cells. Straight dotted black lines drawn across bars of each graph illustrate the expected block at 50% if the compounds acted in a simple additive manner. Simple additive behavior was observed in blends of CBGA and each of: CBGVA, THCA, CBCA, CBD, CBNA, CBN, CBND, and CBL (see, e.g., FIGS. 10E-10L). In some cases (e.g., as shown in FIGS. 10A-10D), combinations of CBGA and another cannabinoid compound (e.g., CBG, CBGV, THCVA, or THCV) showed stronger than predicted inhibitions (e.g., supra-additive combinatory effect) of store-operated calcium entry. In some cases (e.g., as shown in FIGS. 10M-10T) show cannabinoids (e.g., selected from CBDA, CBDVA, CBDV, CBLA, Δ8-THC, and CBCV) that inhibit less than predicted when paired with CBGA in SOCE inhibition experiments performed in Jurkat cells (e.g., sub-additive combinatory effects). Combinations of CBGA and another cannabinoid (e.g., selected from CBDA, CBGVA, THCA, THCVA, and CBNA) tested in THP-1 cells showed sub-additive combinatory effects (see, FIGS. 10U-10Y).

Additional isobolographic analysis of combinatory effects of cannabinoids having SOCE IC₅₀ values greater than 30 μM were performed in THP-1 cells. In these experiments, candidate cannabinoid extracts (selected from Δ8-THC, CBN, CBD, CBG, and CBGV) were administered at 10 μM alone or in combination with 2.2 μM CBGA, which is a concentration near the SOCE IC₅₀ value of CBGA (see FIG. 10Z). The combination of 2.2 μM CBGA and 10 μM of any of CBN, CBG, and CBGV resulted in SOCE inhibition values greater than 50%, indicating that CBN, CBG, and CBGV are simple additive agonists, e.g., when paired with CBGA. Experimental results showed that Δ8-THC and CBD did not increase SOCE inhibition past 50% when paired with 2.2 μM CBGA, indicating that Δ8-THC and CBD are sub-additive (e.g., inhibitory), for example, when paired with 2.2 μM CBGA.

A partial agonist or antagonistic (e.g., sub-additive) effect by a first cannabinoid (e.g., CBDA) may be important in reducing or otherwise modulating side effects that may result from treatment with a second cannabinoid (e.g., CBGA), for example, which may be co-administered with the first cannabinoid. In addition, the ability of other cannabinoids to modulate the physiological or therapeutic activity of, for example, CBGA, may be useful in optimizing an appropriate level or degree of therapeutic response when needed.

Effects observed in these experiments may suggest chemical structure-activity relationships and some selectivity over non-immune cells. In addition, combined administration of minor and major cannabinoids support potential combinatory effects. While high-THC and high-CBD cannabis extracts were active against SOCE in immune cells in these experiments, their activity was fully or partially carried out by components that are not THC or CBD.

FIGS. 10A-B. Combinatory effect of CBGA and other cannabinoids. (10A) Representative SOCE signals in the presence of various cannabinoids, individually (black traces) or in combination with CBGA (red traces). CBGA was used at 2 μM and all other cannabinoids were tested at 3 μM. SOCE was solicited in intact cells by applying 1 μM thapsigargin (Tg). (10B) The bar graph displays SOCE amplitudes calculated from SOCE signals in (A) and normalized to SOCE in the presence of gadolinium. All the data shown here are average of 3 independent runs and the values are mean±sem.

### Example 9

### Effects on the TRPM7 Pathway

This example shows effects of treatment with cannabinoids, such as CBGA, on cell physiology, for example, as it pertains to the TRPM7 pathway. Receptor agonists stimulate receptors (R) and G proteins (G), resulting in activation of phospholipase C (PLC), which produces the second messenger inositol 1,4,5-trisphosphate (IP₃) and causes the release of Ca²⁺ from the endoplasmic reticulum (ER) through IP receptors (IP₃R). This store depletion of Ca²⁺ is sensed by STIM molecules in the (ER), which then couple to and open calcium release-activated calcium (CRAC) channels in the plasma membrane (PM). The ensuing store-operated calcium entry (SOCE) causes a long-lasting increase in intracellular calcium concentration that can cause production and release of inflammatory cytokines as well as cell proliferation (e.g. cancer and fibrosis). TRPM7 is a dual-function protein with both ion channel and kinase activities. It is found both in the plasma and ER membranes and participates in calcium signaling and SOCE in several ways. The ion channel function enables Ca²⁺ and Mg²⁺ influx and helps filling the ER store with Ca²⁺. The kinase function can phosphorylate targets that enhance GPCR signaling to promote Ca²⁺ release and store depletion as well STIM signaling to enable and promote SOCE. Therefore, blocking the kinase activity by, for example, treatment with CBGA would suppress STIM coupling to CRAC channels and indirectly reduce SOCE, in many cases.

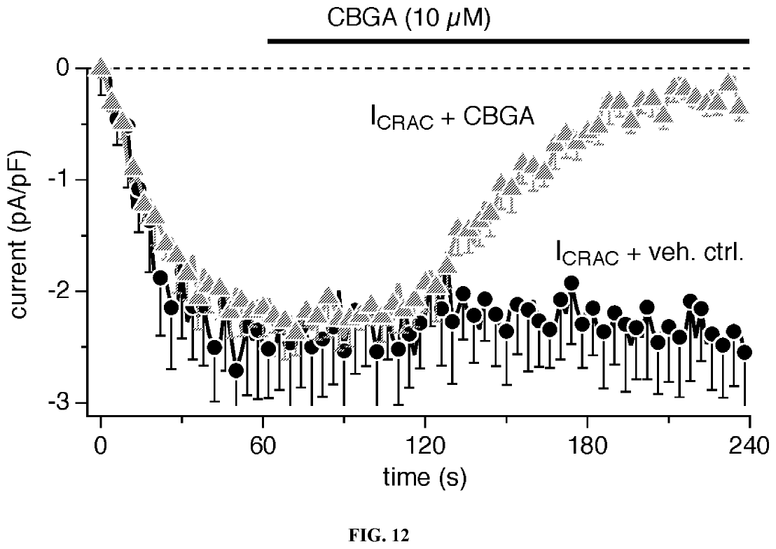

The ability of CBGA to inhibit store-operated calcium entry (SOCE) within cells was experimentally interrogated (see FIG. 12). Jurkat cells were perfused with 50 μM IP₃ to induce store depletion and activation of CRAC channels (the resulting Ca²⁺ inward current is known as I_{CRAC}). I_{CRAC} is a long-lasting inward current at −120 mV membrane potential (black trace) that can be blocked by 10 μM CBGA (gray trace). FIG. 12 shows treatment with 10 μM CBGA completely blocks the inward calcium current (gray trace), while treatment with a vehicle control (veh. ctrl.) does not affect IP₃-induced I_{CRAC} (black trace).

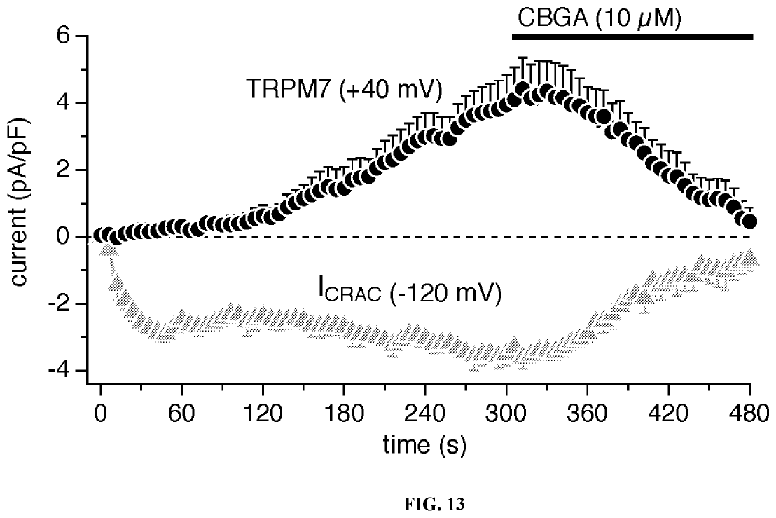

FIG. 13 shows an experiment using the same protocol and cell type (Jurkat) as in FIG. 12 over a longer period of time. Inward CRAC currents were induced at −120 mV (gray trace) and outward currents at +40 mV (black trace, where CRAC currents reverse and are essentially absent). At the +40 mV voltage potential, TRPM7 channels produce monovalent outward currents. Perfusion of 50 μM IP3 activates I_{CRAC} at −120 mV as above and the removal of intracellular ATP slowly activates TRPM7 current at +40 mV. Application of 10 μM CBGA blocks both outward TRPM7 and inward CRAC currents (see FIG. 13, black and gray traces, respectively). Without being bound by theory, CBGA may also block TRPM7's kinase activity, similar to other TRPM7 blockers such as NS8593.

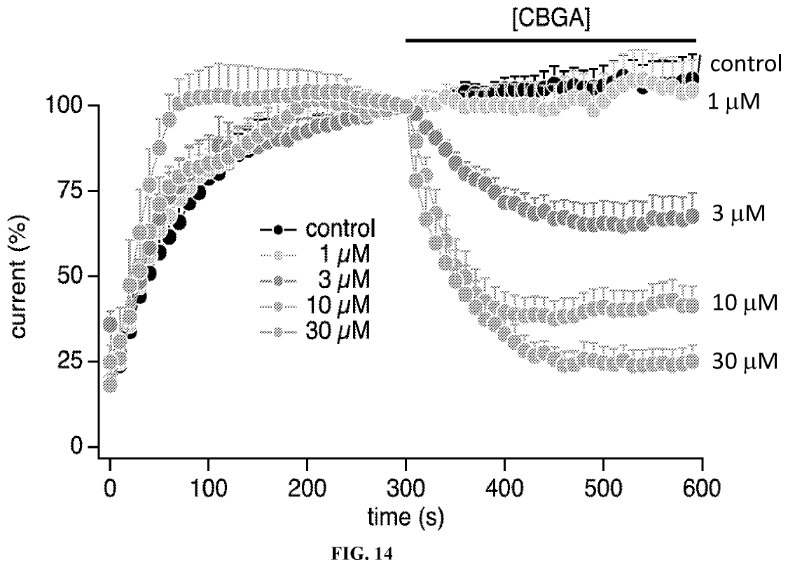

Activation of TRPM7 over-expressed in HEK293 cells by perfusing the cells with intracellular solution containing 0 ATP and 0 Mg²⁺, resulting in fast and maximal activation of TRPM7 outward currents at +40 mV is shown in FIG. 14. Application of 0 μM (control), 1 μM, 3 μM, 10 μM, or 30 μM CBGA causes dose-dependent block of TRPM7 current.

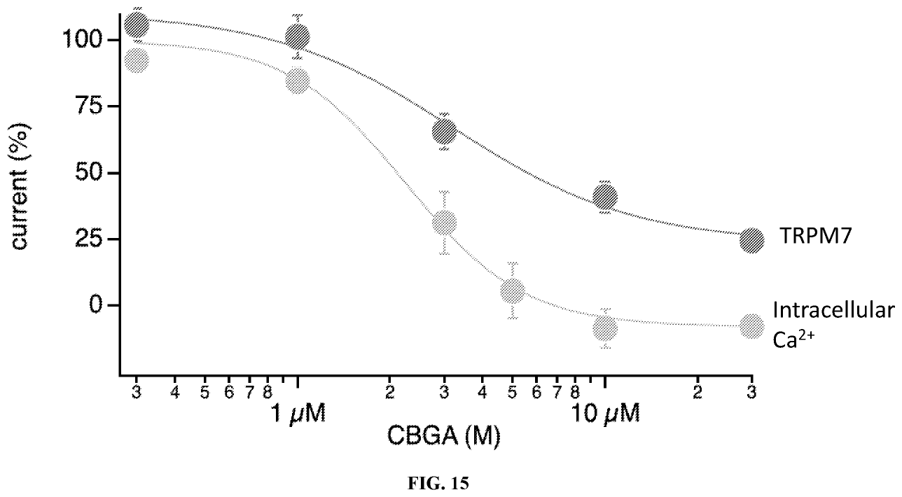

Data from dose-response curves for the inhibition of TRPM7 currents (dark gray symbols) obtained in experiments shown in FIG. 14 and SOCE-mediated increases in intracellular Ca²⁺ (light gray symbols) are shown in FIG. 15. Results from these experiments show that CBGA blocks both mechanisms with similar low micromolar IC₅₀ values of about 3 μM and 2 μM, respectively.

### Example 10

### Effects of Heat Treatment Cannabinoid Extracts

This example shows effects of temperature on store-operated calcium entry (SOCE) in various cell types.

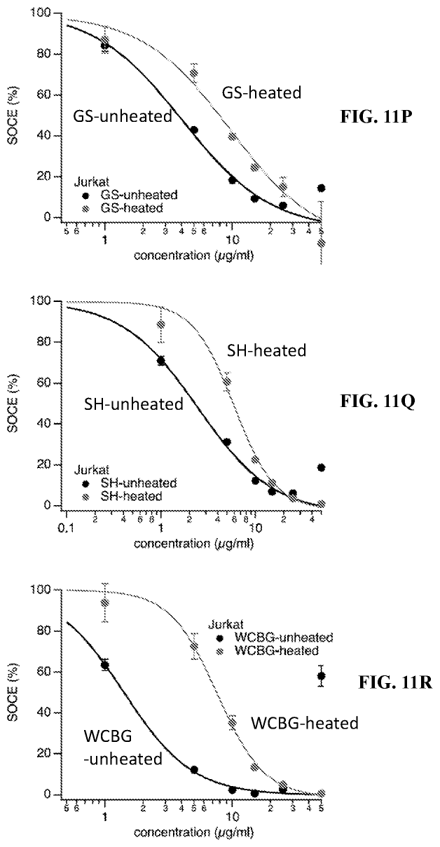

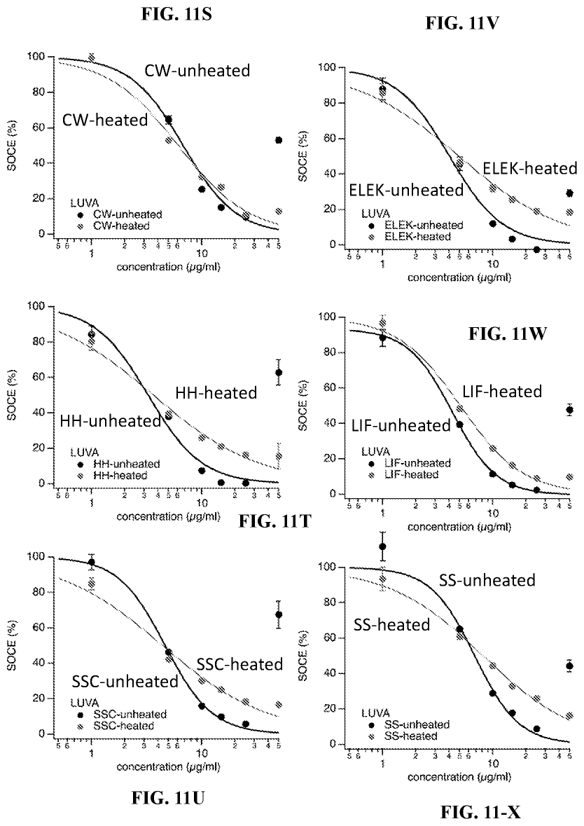

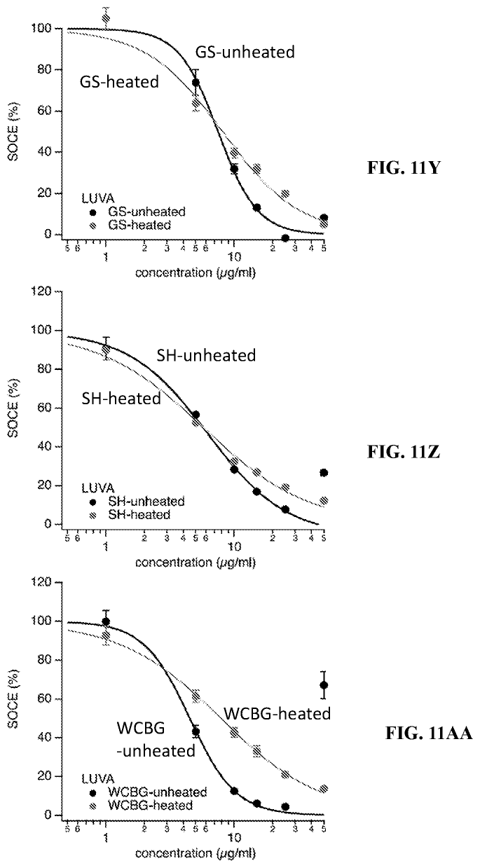

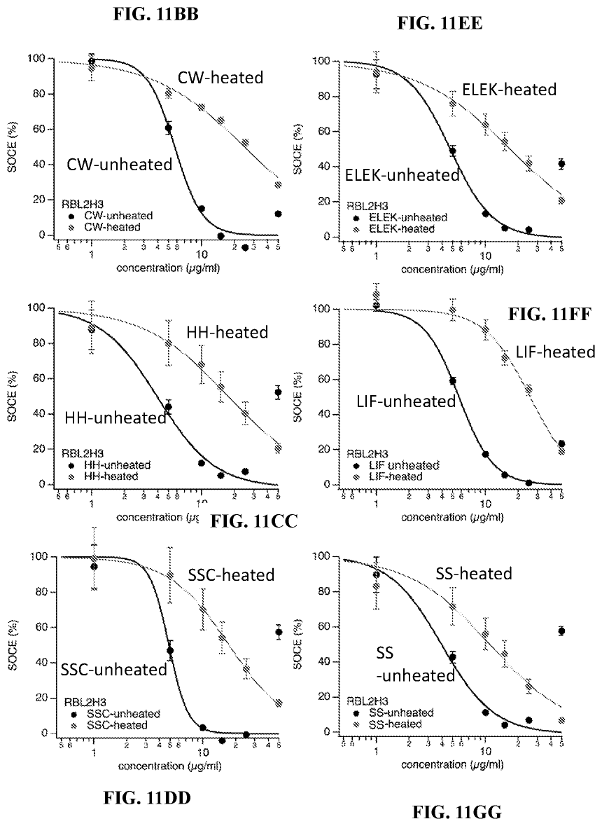

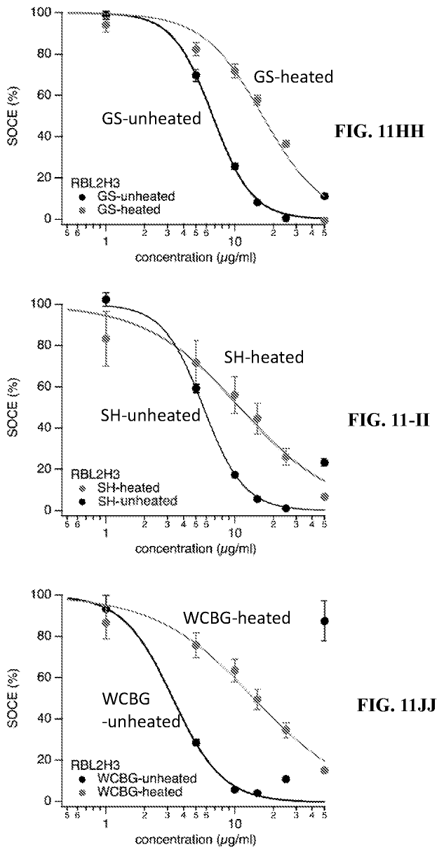

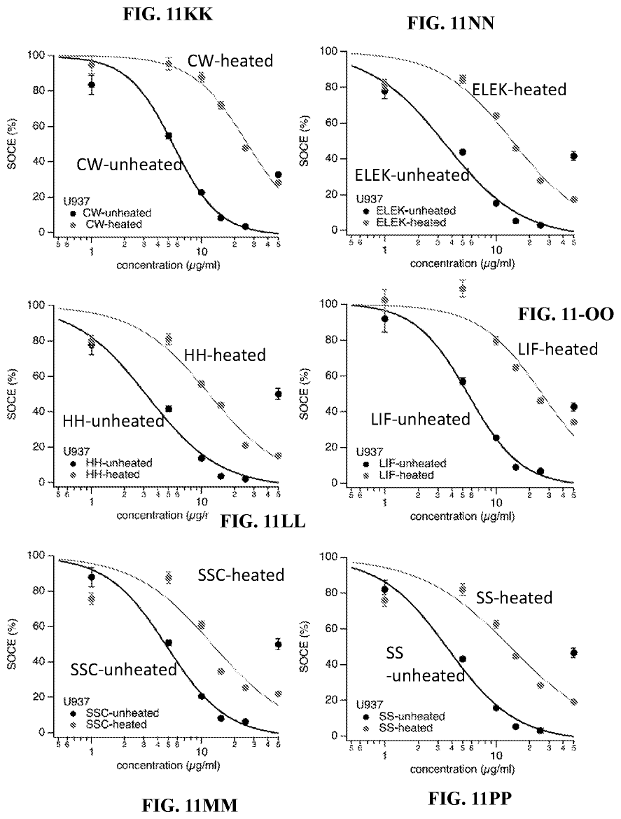

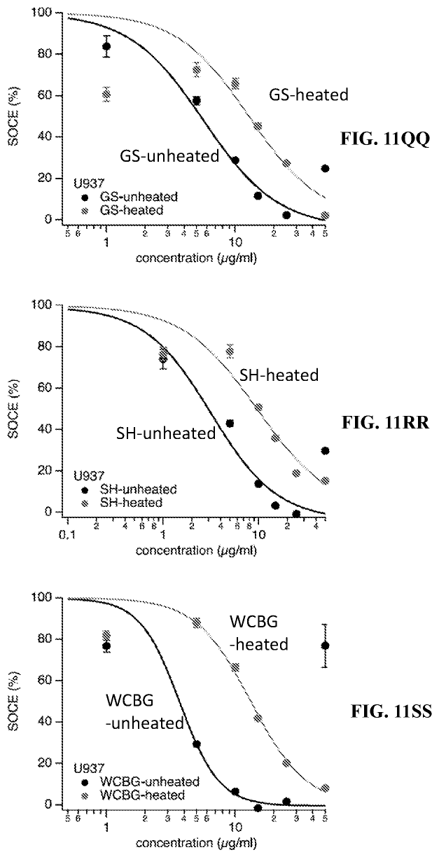

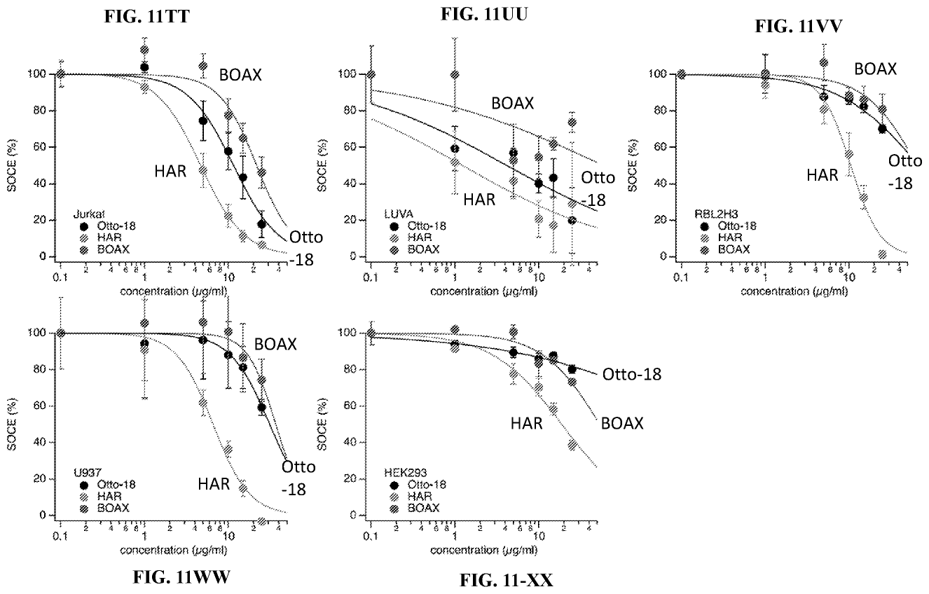

FIGS. 11A-11SS show dose-response curves for hemp varieties on store-operated calcium entry (SOCE) induced by thapsigargin in HEK-293 cells (FIGS. 11A-11-I), Jurkat cells (FIGS. 11J-11R), LUVA cells (FIGS. 11S-11AA), RBL-2H3 cells (FIGS. 11BB-11JJ), U937 (FIGS. 11KK-11SS). Briefly, SOCE inhibition experiments were performed as described in Example 6, using each of the hemp extracts listed below and in Tables 6 and 7 at six concentrations each in each of the five cell types listed above to obtain dose response curves, as shown in FIGS. 11A-11SS. The effects on SOCE amplitude were plotted in a dose-dependent manner to determine the IC₅₀ for each tested extract. IC₅₀ values are compiled in Table 6 and Table 7 for unheated and heated experiments, respectively. All data are averages of three independent runs±SEM. Hemp extracts were either extracted at room temperature (“unheated”, black data points) or exposed, after extraction, to an increased temperature of 115° C. for 60 min (“heated”, gray data points) and were tested at 6 different doses from 1 to 50 μg/ml. Hemp extracts used in FIGS. 11A-11SS are: Sour Space Candy (SSC), Hawaiian Haze (HH), Special Sauce (SS), Suver Haze (SH), White CBG (WCBG), Elektra (ELEK), Cherry Wine (CW), Lifter (LIF), Grape Soda (GS). Results from these experiments indicate that IC₅₀ of hemp extracts can be modulated by adjusting the temperature to which cannabinoids are exposed during extraction or thereafter. For example, these results indicate that increasing temperature can increase the IC₅₀ value of cannabinoids for inhibiting store-operated calcium entry in human cells. Furthermore, the data show that the tested extracts had the most potent effect on SOCE in Jurkat cells, when extracts were unheated (Table 6). When heated, the tested extracts were most potent in Jurkat and Luva cells (Table 7).

FIGS. 11TT-11-XX show dose-response curves for hemp varieties on store-operated calcium entry (SOCE) induced by thapsigargin in HEK-293, Jurkat, U937, LUVA, and RBL-2H3 cells. Hemp extracts (cold extracted and unheated) were tested at 6 different doses from 1 to 50 μg/ml. The effects on SOCE amplitude were plotted in a dose-dependent manner to determine the IC₅₀ for each tested compound (see Table 6 for determined IC₅₀ values). All data are averages of three independent runs±SEM. Tested hemp varieties were: Otto-18, Harlequin (HAR), and BOAX. Results showed that Harlequin had the lowest IC₅₀ values, indicating that Harlequin was the most potent variety of the three tested varieties.

TABLE 6
describes IC₅₀ values (μg/mL) for heat treated and non-heated samples.

| IC₅₀ (μg/ml) | S01 | S02 | S03 | S04 | S05 | S06 | S07 | S08 | S09 |
|---|---|---|---|---|---|---|---|---|---|
| Batch #1 | 1.74 | 1.63 | 2.07 | 1.89 | 1.10 | 1.54 | 1.09 | 1.94 | 1.73 |
| Batch #2 | 4.63 | 4.59 | 4.81 | 4.33 | 1.42 | 1.66 | 1.32 | 4.67 | 10.41 |
| Batch #2 heated | 17.38 | 15.34 | 8.80 | 9.20 | 7.51 | 5.47 | 6.33 | 10.71 | 22.49 |
| | | | | CBD | | | | | |
| | IC₅₀ (μg/ml) | S10 | S11 | Ext | 118-4 | 118-6 | Otto18 | Har | Boax |
| | Batch #1 | 5.90 | 2.07 | 5.13 | 9.76 | 8.99 | 11.55 | 4.19 | 21.84 |
| | Batch #2 | 3.62 | 8.08 | | | | 9.36 | 2.07 | 14.21 |
| | Batch #2 heated | 48.65 | 14363.00 | | | | 11.53 | 6.34 | 10.12 |

TABLE 7
describes IC₅₀ values (μg/mL) for select hemp samples before
((−) heat treatment) and after ((+) heat treatment) heat treatment.
As seen, the non-heated treated hemp samples are generally more
potent than heat treated samples, with IC₅₀ levels increasing
substantially with heat-treatment.

| | | | | | |
|---|---|---|---|---|---|
| IC₅₀ (ug/mL) | Jurkat | U937 | Luva | RBL-2H3 | HEK293 |

(−) HEAT TREATMENT

| | | | | | |
|---|---|---|---|---|---|
| Sour Space Candy | 2.54 | 4.86 | 4.65 | 4.88 | 13.15 |
| Hawaiian Haze | 2 | 3.22 | 3.26 | 4 | 8.23 |
| Special Sauce | 2.52 | 3.72 | 6.85 | 3.96 | 8.86 |
| Suver Haze | 2.44 | 3.18 | 6.19 | 4.5 | 7.95 |
| White CBG | 1.4 | 3.7 | 4.5 | 3.31 | 7.86 |
| Elektra | 2.3 | 3.52 | 4.05 | 4.76 | 10.18 |
| Cherry Wine | 6.27 | 5.57 | 6.79 | 5.72 | 12.17 |
| Lifter | 3.48 | 5.63 | 4.28 | 5.72 | 12.66 |
| Grape Soda | 4 | 5.67 | 7.49 | 6.74 | 16.51 |

(+) HEAT TREATMENT

| | | | | | |
|---|---|---|---|---|---|
| Sour Space Candy | 5.64 | 12.45 | 4.41 | 4.88 | 17.56 |
| Hawaiian Haze | 6.23 | 11.71 | 4.41 | 17.58 | 44.85 |
| Special Sauce | 6.66 | 13.3 | 8.18 | 14.49 | 47.03 |
| Suver Haze | 5.95 | 9.62 | 5.74 | 10.96 | 21.19 |
| White CBG | 7.61 | 13.2 | 7.85 | 14.16 | 19.76 |
| Elektra | 6.69 | 14.02 | 4.93 | 16.79 | 33.81 |
| Cherry Wine | 7.61 | 26.23 | 6.12 | 24.67 | 42.24 |
| Lifter | 6.47 | 25.85 | 5.06 | 25.73 | 41.61 |
| Grape Soda | 9.45 | 12.77 | 8.12 | 16.51 | 39.52 |

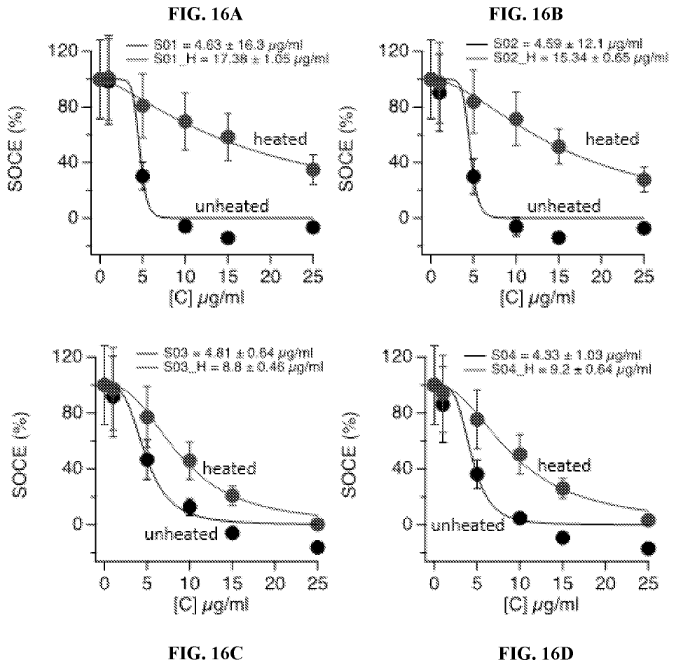

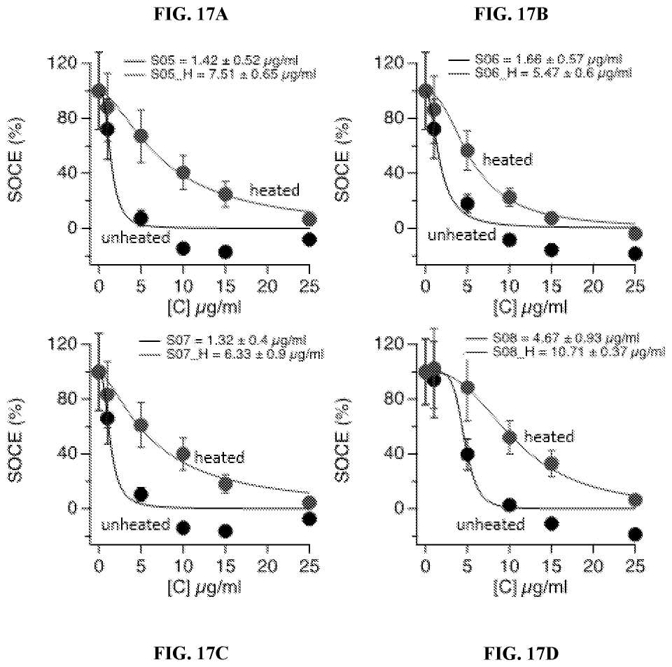

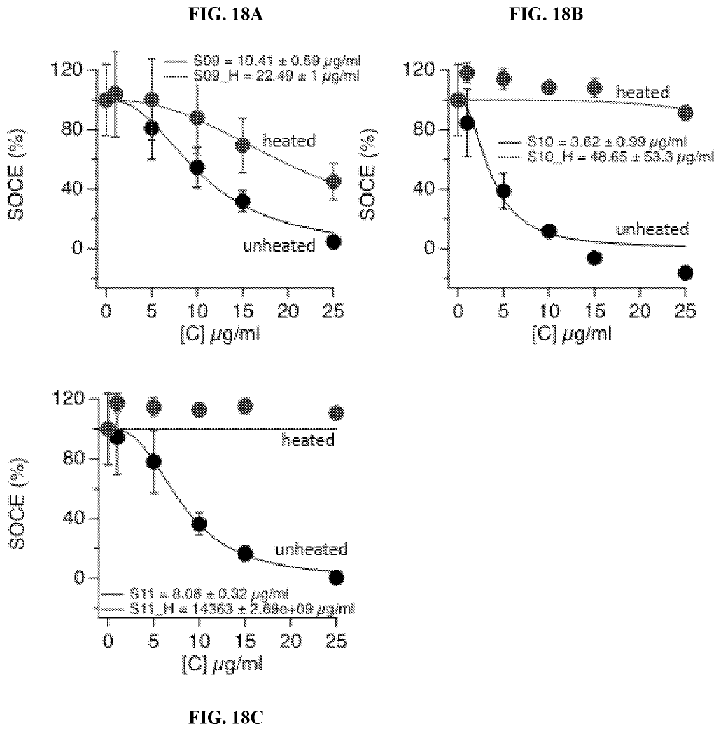

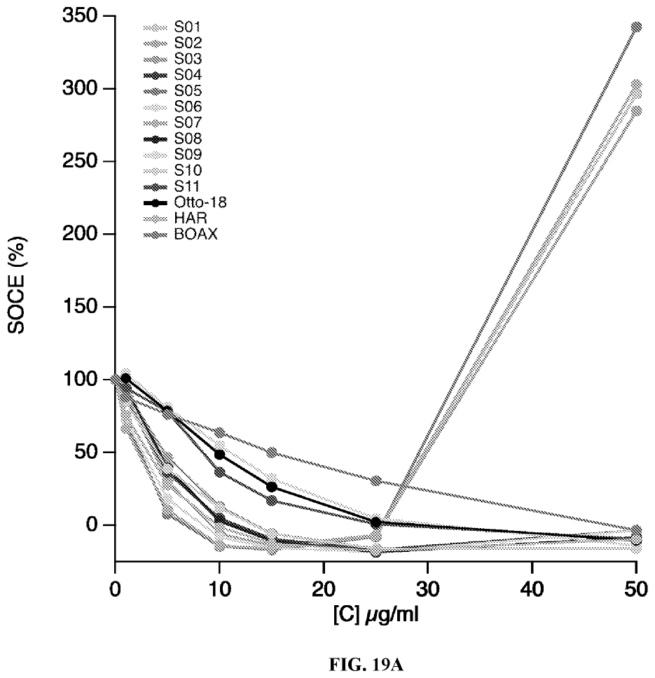

FIGS. 16A-19B illustrate activity for extracts S01-S11 and unfractionated extracts of strains Otto-18, HAR, and BOAX with and without heat treatment. Extracts S01, S02, S05, and S07 had increased Ca²⁺ channel blocking after heat treatment.

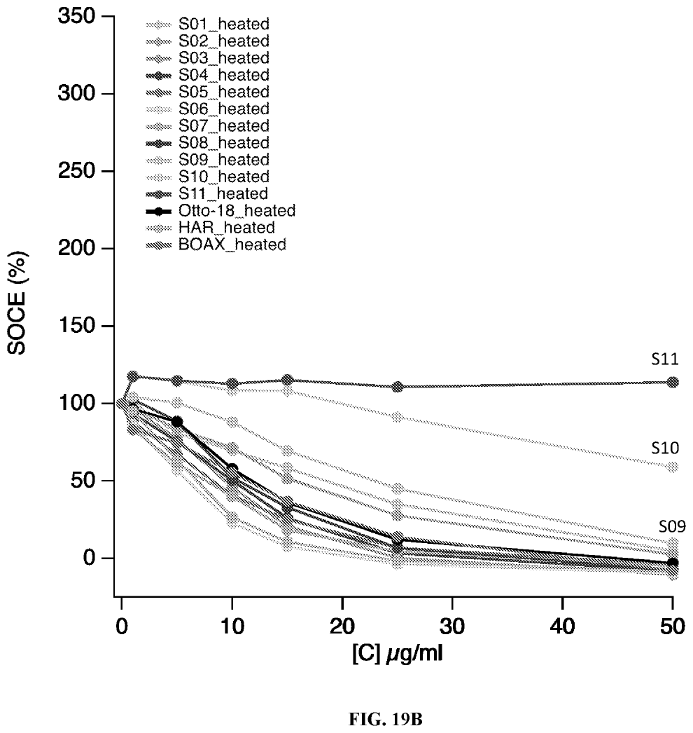

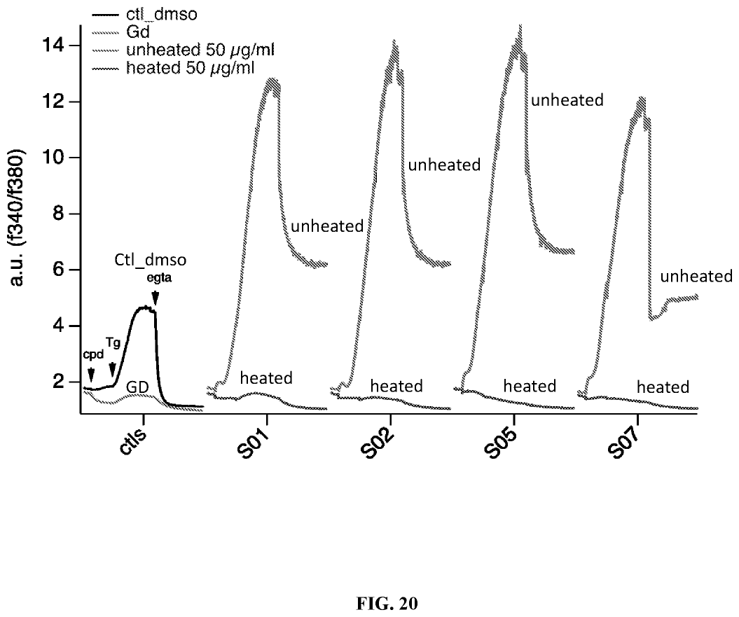

FIG. 20 illustrates SOCE activity for extracts S01, S02, S05, and S07 with and without heat treatment. DMSO was used as a negative control, and gadolinium was used as a positive control. Unheated extracts resulted in blocking of calcium channels, similar to the positive control gadolinium (FIGS. 19A and 19B). At the highest dosage measured of 50 μg/ml, some heated extracts (S01, S02, S05, and S07) resulted in blocking of calcium channels, although potency was decreased at lower dosages (FIG. 20).

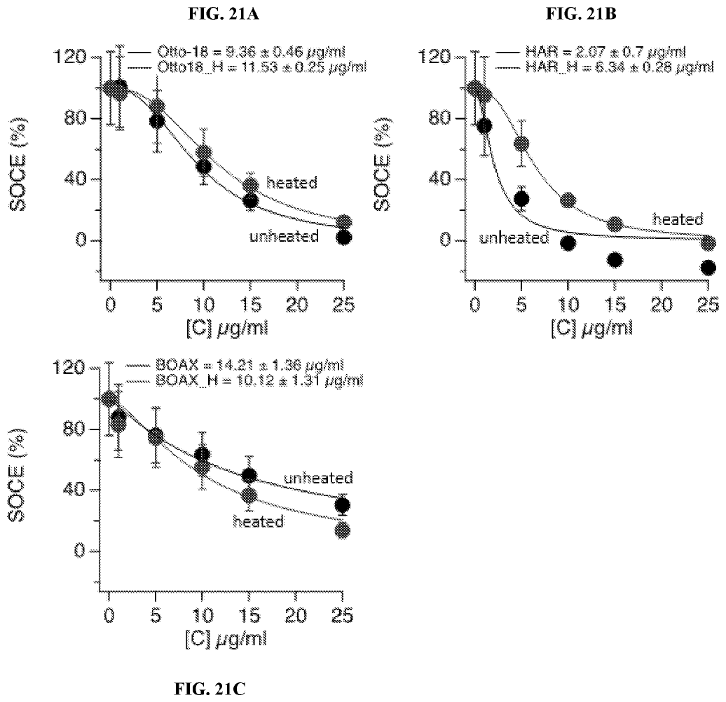

FIG. 21A-12C illustrates SOCE activity for unfractionated extracts of cannabis strains Otto-18, HAR (harlequin), and BOAX with and without heat treatment.

### Example 11

### Effects of Unheated, Heat-Treated and Combined Unheated and Heat-Treated Hemp Extracts

The general extraction method of Example 6 was carried out, and hemp extracts were subjected to heat treatment prior to testing for activity in the SOCE assay. In some instances, unheated and heated hemp extracts were combined prior to activity testing in the SOCE assay.

Table 8 describes IC₅₀ values (μg/mL) for select hemp extract samples before ((−) heat treatment), after ((+) heat treatment) heat treatment, and combined (1:1) unheated and heated hemp extract samples. In some instances, the combined unheated and heated hemp extract samples were less potent than treating with unheated or heat-treated hemp samples alone.

| | | | |
|---|---|---|---|
| | | | Combined |
| | (−) heat | (+) heat | (−)/(+) heat |
| IC₅₀ (ug/mL) | treatment | treatment | treatment |

JURKAT

| | | | |
|---|---|---|---|
| Sour Space Candy | 2.54 | 5.64 | 4.67 |
| Hawaiian Haze | 2 | 6.23 | 4.84 |
| Special Sauce | 2.52 | 6.66 | 1.32 |
| Suver Haze | 2.44 | 5.95 | 2.49 |
| White CBG | 1.4 | 7.61 | 4.71 |
| Elektra | 2.3 | 6.69 | 5 |
| Cherry Wine | 6.27 | 7.61 | 4.96 |
| Lifter | 3.48 | 6.47 | 4.88 |
| Grape Soda | 4 | 9.45 | 5.08 |

RBL2H3

| | | | |
|---|---|---|---|
| Sour Space Candy | 4.88 | 4.88 | 7.22 |
| Hawaiian Haze | 4 | 17.58 | 8.72 |
| Special Sauce | 3.96 | 14.49 | 5.48 |
| Suver Haze | 4.5 | 10.96 | 6.33 |
| White CBG | 3.31 | 14.16 | 5.36 |
| Elektra | 4.76 | 16.79 | 9.92 |
| Cherry Wine | 5.72 | 24.67 | 8.99 |
| Lifter | 5.72 | 25.73 | 9 |
| Grape Soda | 6.74 | 16.51 | 9.74 |

U937

| | | | |
|---|---|---|---|
| Sour Space Candy | 4.86 | 12.45 | 5.88 |
| Hawaiian Haze | 3.22 | 11.71 | 4.95 |
| Special Sauce | 3.72 | 13.3 | 5.94 |
| Suver Haze | 3.18 | 9.62 | 6.6 |
| White CBG | 3.7 | 13.2 | 5.65 |
| Elektra | 3.52 | 14.02 | 6.74 |
| Cherry Wine | 5.57 | 26.23 | 9.42 |
| Lifter | 5.63 | 25.85 | 9.36 |
| Grape Soda | 5.67 | 10 | 11.29 |

LUVA

| | | | |
|---|---|---|---|
| Sour Space Candy | 4.65 | 4.41 | 3.39 |
| Hawaiian Haze | 3.26 | 4.41 | 3.97 |
| Special Sauce | 6.85 | 8.18 | 5.32 |
| Suver Haze | 6.19 | 5.74 | 5.87 |
| White CBG | 4.5 | 7.85 | 4.76 |
| Elektra | 4.05 | 4.93 | 4.79 |
| Cherry Wine | 6.79 | 6.12 | 4.83 |
| Lifter | 4.28 | 5.06 | 3.69 |
| Grape Soda | 7.49 | 8.12 | 6.15 |

HEK293

| | | | |
|---|---|---|---|
| Sour Space Candy | 13.15 | 17.56 | 23.14 |
| Hawaiian Haze | 8.23 | 44.85 | 22.2 |
| Special Sauce | 8.86 | 47.03 | 18.33 |
| Suver Haze | 7.95 | 21.19 | 20.81 |
| White CBG | 7.86 | 19.76 | 12.4 |
| Elektra | 10.18 | 33.81 | 24.08 |
| Cherry Wine | 12.17 | 42.24 | 18.1 |
| Lifter | 12.66 | 41.61 | 14.92 |
| Grape Soda | 16.51 | 39.52 | 16.94 |

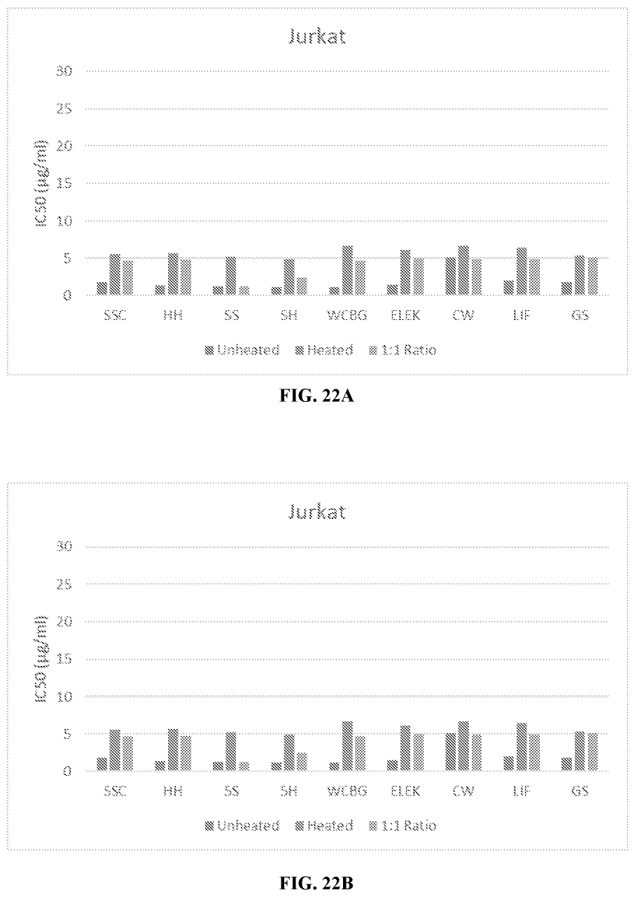

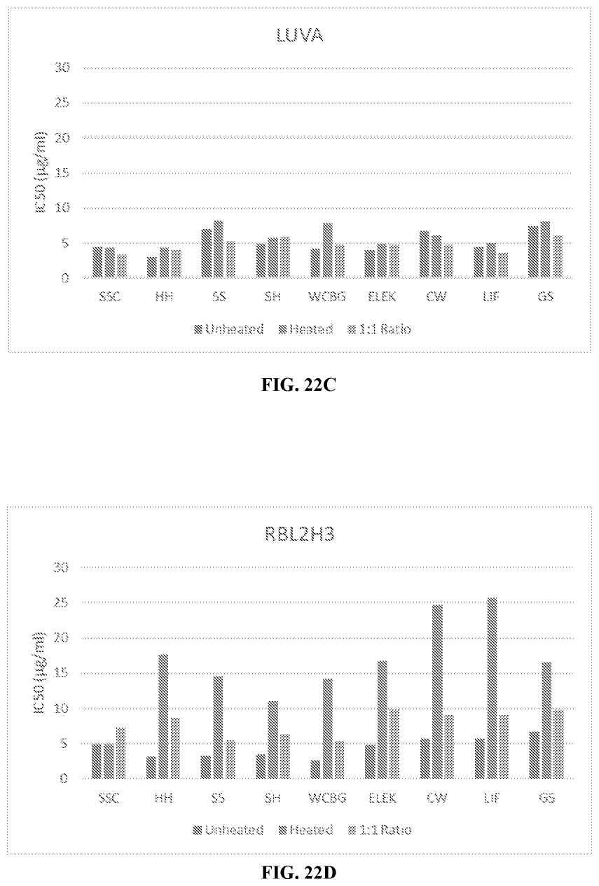

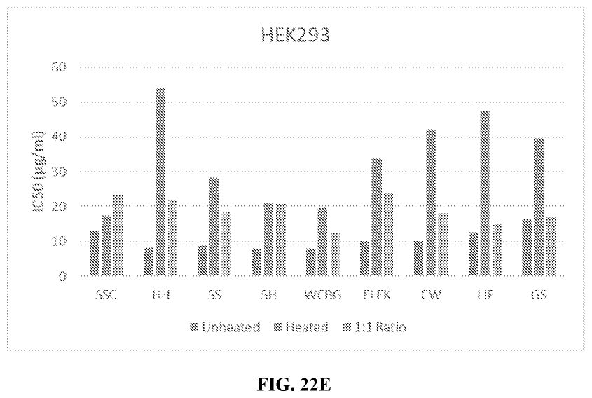

FIGS. 22A-22E illustrate SOCE activity for hemp extracts with and without heat treatment and combined (1:1) unheated and heated hemp extracts in different cell types. FIG. 22A, Jurkat cells; FIG. 22B, RBL2H3 cells; FIG. 22C, U937 cells; FIG. 22D, Luva cells; FIG. 22E, HEK293 cells. As seen, unheated hemp extracts are generally more potent in SOCE assays than heated extracts. In most cases, the heating appears to reduce potency (increasing IC₅₀). However, the mixtures show fairly complex behavior, where the mixtures are in most cases more active than the heated extracts alone, but generally less active than the unheated extracts. In some instances, the mixtures appear to have even less activity than the additive effect of the unheated and heated extracts alone, indicating a possible inhibitory effect of the extracts.

While preferred embodiments have been shown and described herein, such embodiments are provided by way of example only. Numerous variations, changes, and substitutions will occur to those skilled in the art without departing from the disclosed embodiments. It should be understood that various alternatives to the embodiments described herein may be employed in practicing the embodiments without departing from the spirit or scope of the invention.

What is claimed is:

1. A pharmaceutical composition for the treatment or amelioration of a disease in a patient in need thereof, the pharmaceutical composition comprising: at least one acidic cannabinoid compound, at least one non-acidic cannabinoid compound and a pharmaceutical excipient.

2. The pharmaceutical composition of claim 1, wherein the acidic cannabinoid compound and non-acidic cannabinoid compound are extracted from a cannabis plant.

3. The pharmaceutical composition of claim 1, wherein the acidic cannabinoid compound and non-acidic cannabinoid compound are synthesized.

4. The pharmaceutical composition of claim 1, wherein the acidic cannabinoid compound is extracted from a cannabis plant.

5. The pharmaceutical composition of claim 1, wherein the acidic cannabinoid compound is derived from an extract from a cannabis plant.

6. The pharmaceutical composition of claim 4, wherein the extraction process is chosen from the group consisting of supercritical fluid extraction, solvent extraction, ultrasound-assisted extraction, or combinations thereof.

7. The pharmaceutical composition of claim 6, wherein the solvent is an alcohol, hexane or combinations thereof.

8. The pharmaceutical composition of claim 7, wherein the alcohol is ethanol or isopropanol.

9. The pharmaceutical composition of claim 6, wherein the extraction process does not decarboxylate the acidic cannabinoid compounds in the plant extract.

10. The pharmaceutical composition of claim 6, wherein the extraction process yields an acidic cannabinoid compound of about 90% purity, about 95% purity or about 99% purity.

11. The pharmaceutical composition of claim 1, wherein the non-acidic cannabinoid compound is extracted from a cannabis plant.

12. The pharmaceutical composition of claim 1, wherein the non-acidic cannabinoid compound is derived from an extract from a cannabis plant.

13. The pharmaceutical composition of claim 11, wherein the extraction process is chosen from the group consisting of supercritical fluid extraction, solvent extraction, ultrasound-assisted extraction, microwave-assisted extracted or combinations thereof.

14. The pharmaceutical composition of claim 11, wherein the cannabis plant extract is processed to decarboxylate acidic-cannabinoid compounds in the cannabis plant extract.

15. The pharmaceutical composition of claim 14, wherein the decarboxylation process includes a heating step.

16. The pharmaceutical composition of claim 15, wherein the heating step is maintained at above 100° C. for a predetermined time period.

17. The pharmaceutical composition of claim 14, wherein the extraction and decarboxylation process yields a non-acidic cannabinoid compound of about 90% purity, about 95% purity or about 99% purity.

18. The pharmaceutical composition of claim 1, wherein the composition reduces an influx of extracellular Ca²⁺ in a cell as compared to the individual acidic cannabinoid compound and non-acidic cannabinoid compound.

19. The pharmaceutical composition of claim 1, wherein the acidic cannabinoid compound is CBDA, CBGA, THCA, CBDVA, CBGVA, CBLA, CBNA, CBCA, THCVA, or combinations thereof.

20. The pharmaceutical composition of claim 19, wherein the composition comprises at least two acidic cannabinoid compounds.

21. The pharmaceutical composition of claim 19, wherein the acidic cannabinoid compound is added in a purified form.

22. The pharmaceutical composition of claim 19, wherein the acidic cannabinoid compound is added in a non-purified form.

23. The pharmaceutical composition of claim 1, wherein the non-acidic cannabinoid compound is CBD, CBG, THC, CBDV, CBGV, CBL, CBN, CBC, THCV, or combinations thereof.

24. The pharmaceutical composition of claim 23, wherein the composition comprises at least two non-acidic cannabinoid compounds.

25. The pharmaceutical composition of claim 23, wherein the non-acidic cannabinoid compound is added in a purified form.

26. The pharmaceutical composition of claim 23, wherein the non-acidic cannabinoid compound is added in a non-purified form.

27. The pharmaceutical composition of claim 1, wherein at least one acidic cannabinoid compound and at least one non-acidic cannabinoid compound is present at a molar ratio of between 1:1 and 1:200.

28. The pharmaceutical composition of claim 1, wherein at least one non-acidic cannabinoid compound and at least one acidic cannabinoid compound is present at a molar ratio of between 1:1 and 1:200.

29. The pharmaceutical composition of claim 1, wherein at least one acidic cannabinoid compound is present in an extract of a cannabis plant, and at least one non-acidic cannabinoid compound is present in an extract of a cannabis plant.

30. The pharmaceutical composition of claim 29, wherein the extract comprising at least one acidic cannabinoid compound and the extract comprising at least one non-acidic cannabinoid compound is combined at a ratio of between 1:1 and 1:200.

31. The pharmaceutical composition of claim 29, wherein the extract comprising at least one non-acidic cannabinoid compound and the extract comprising at least one acidic cannabinoid compound is combined at a ratio of between 1:1 and 1:200.

32. The pharmaceutical composition of claim 1, wherein the composition treats or ameliorates an autoimmune disease, a neurodegenerative disease, an inflammatory condition, pain, cancer or combinations thereof.

33. The pharmaceutical composition of claim 1, wherein the acidic cannabinoid is CBGA.

34. The pharmaceutical composition of claim 1, wherein the non-acidic cannabinoid is CBG, CBD, CBDV, THC or combinations thereof.

35. The pharmaceutical composition of claim 1, wherein the pharmaceutical excipient is chosen from the group consisting of buffer, diluent, disintegrant, glidant, lubricant, coating, carrier, controlled release agent, adjuvant, vehicle, binder, emulsifying agent, or combinations thereof.

36. A pharmaceutical composition for the treatment or amelioration of an autoimmune disease comprising: at least one acidic cannabinoid compound, at least one non-acidic cannabinoid compound and a pharmaceutical excipient.

37. The pharmaceutical composition of claim 36, wherein the acidic cannabinoid compound is CBDA, CBGA, THCA, CBDVA, CBGVA, CBLA, CBNA, CBCA, THCVA, or combinations thereof.

38. The pharmaceutical composition of claim 36, wherein the composition comprises at least two acidic cannabinoid compounds.

39. The pharmaceutical composition of claim 36, wherein the acidic cannabinoid compound is added in a purified form.

40. The pharmaceutical composition of claim 36, wherein the non-acidic cannabinoid compound is CBD, CBG, THC, CBL, CBCV or combinations thereof.

41. The pharmaceutical composition of claim 36, wherein the composition comprises at least two non-acidic cannabinoid compounds.

42. The pharmaceutical composition of claim 36, wherein the non-acidic cannabinoid compound is added in a purified form.

43. The pharmaceutical composition of claim 36, wherein at least one acidic cannabinoid compound and at least one non-acidic cannabinoid compound is present at a molar ratio of between 1:1 and 1:200.

44. The pharmaceutical composition of claim 36, wherein at least one non-acidic cannabinoid compound and at least one acidic cannabinoid compound is present at a molar ratio of between 1:1 and 1:200.

45. A method of treating or ameliorating an autoimmune disease, the method comprising administering to a patient in need thereof a therapeutically effective amount of a pharmaceutical composition of any one of claims 36-44, the pharmaceutical composition comprising at least one acidic cannabinoid compound, at least one non-acidic cannabinoid compound and a pharmaceutical excipient.

46. A pharmaceutical composition for the treatment or amelioration of cancer comprising at least one acidic cannabinoid compound, at least one non-acidic cannabinoid compound and a pharmaceutical excipient.

47. The pharmaceutical composition of claim 46, wherein the acidic cannabinoid compound is CBDA, CBGA, THCA, CBDVA, CBGVA, CBLA, CBNA, CBCA, THCVA, or combinations thereof.

48. The pharmaceutical composition of claim 46, wherein the composition comprises at least two acidic cannabinoid compounds.

49. The pharmaceutical composition of claim 46, wherein the acidic cannabinoid compound is added in a purified form.

50. The pharmaceutical composition of claim 46, wherein the non-acidic cannabinoid compound is CBD, CBG, THC, CBDV, CBGV, CBL, CBN, CBC, THCV, or combinations thereof.

51. The pharmaceutical composition of claim 46, wherein the composition comprises at least two non-acidic cannabinoid compounds.

52. The pharmaceutical composition of claim 46, wherein the non-acidic cannabinoid compound is added in a purified form.

53. The pharmaceutical composition of claim 46, wherein at least one acidic cannabinoid compound and at least one non-acidic cannabinoid compound is present at a molar ratio of between 1:1 and 1:200.

54. The pharmaceutical composition of claim 46, wherein at least one non-acidic cannabinoid compound and at least one acidic cannabinoid compound is present at a molar ratio of between 1:1 and 1:200.

55. A method of treating or ameliorating cancer, the method comprising administering to a patient in need thereof a therapeutically amount of a pharmaceutical composition, the pharmaceutical composition of any one of claims 46-54 comprising at least one acidic cannabinoid compound, at least one non-acidic cannabinoid compound and a pharmaceutical excipient.

56. A pharmaceutical composition for the treatment or amelioration of an inflammatory condition comprising: at least one acidic cannabinoid compound, at least one non-acidic cannabinoid compound and a pharmaceutical excipient.

57. The pharmaceutical composition of claim 56, wherein the acidic cannabinoid compound is CBDA, CBGA, THCA, CBDVA, CBGVA, CBLA, CBNA, CBCA, THCVA, or combinations thereof.

58. The pharmaceutical composition of claim 56, wherein the composition comprises at least two acidic cannabinoid compounds.

59. The pharmaceutical composition of claim 56, wherein the acidic cannabinoid compound is added in a purified form.

60. The pharmaceutical composition of claim 56, wherein the non-acidic cannabinoid compound is CBD, CBG, THC, CBDV, CBGV, CBL, CBN, CBC, THCV, or combinations thereof.

61. The pharmaceutical composition of claim 56, wherein the composition comprises at least two non-acidic cannabinoid compounds.

62. The pharmaceutical composition of claim 56, wherein the non-acidic cannabinoid compound is added in a purified form.

63. The pharmaceutical composition of claim 56, wherein at least one acidic cannabinoid compound and at least one non-acidic cannabinoid compound is present at a molar ratio of between 1:1 and 1:200.

64. The pharmaceutical composition of claim 56, wherein at least one non-acidic cannabinoid compound and at least one acidic cannabinoid compound is present at a molar ratio of between 1:1 and 1:200.

65. A method of treating or ameliorating an inflammatory condition, the method comprising administering to a patient in need thereof a therapeutically amount of a pharmaceutical composition of any one of claims 56-65, the pharmaceutical composition comprising at least one acidic cannabinoid compound, at least one non-acidic cannabinoid compound and a pharmaceutical excipient.

66. A pharmaceutical composition for the treatment or amelioration of a neurodegenerative condition comprising: at least one acidic cannabinoid compound, at least one non-acidic cannabinoid compound and a pharmaceutical excipient.

67. The pharmaceutical composition of claim 66, wherein the acidic cannabinoid compound is CBDA, CBGA, THCA, CBDVA, CBGVA, CBLA, CBNA, CBCA, THCVA, or combinations thereof.

68. The pharmaceutical composition of claim 66, wherein the composition comprises at least two acidic cannabinoid compounds.

69. The pharmaceutical composition of claim 66, wherein the acidic cannabinoid compound is added in a purified form.

70. The pharmaceutical composition of claim 66, wherein the non-acidic cannabinoid compound is CBD, CBG, THC, CBDV, CBGV, CBL, CBN, CBC, THCV, or combinations thereof.

71. The pharmaceutical composition of claim 66, wherein the composition comprises at least two non-acidic cannabinoid compounds.

72. The pharmaceutical composition of claim 66, wherein the non-acidic cannabinoid compound is added in a purified form.

73. The pharmaceutical composition of claim 66, wherein at least one acidic cannabinoid compound and at least one non-acidic cannabinoid compound is present at a molar ratio of between 1:1 and 1:200.

74. The pharmaceutical composition of claim 66, wherein at least one non-acidic cannabinoid compound and at least one acidic cannabinoid compound is present at a molar ratio of between 1:1 and 1:200.

75. A method of treating or ameliorating a neurodegenerative disorder, the method comprising administering to a patient in need thereof a therapeutically amount of a pharmaceutical composition of any one of claims 66-74, the pharmaceutical composition comprising at least one acidic cannabinoid compound, at least one non-acidic cannabinoid compound and a pharmaceutical excipient.

76. A pharmaceutical composition for the treatment or amelioration of pain comprising: at least one acidic cannabinoid compound, at least one non-acidic cannabinoid compound and a pharmaceutical excipient.

77. The pharmaceutical composition of claim 76, wherein the acidic cannabinoid compound is CBDA, CBGA, THCA, CBDVA, CBGVA, CBLA, CBNA, CBCA, THCVA, or combinations thereof.

78. The pharmaceutical composition of claim 76, wherein the composition comprises at least two acidic cannabinoid compounds.

79. The pharmaceutical composition of claim 76, wherein the acidic cannabinoid compound is added in a purified form.

80. The pharmaceutical composition of claim 76, wherein the non-acidic cannabinoid compound is CBD, CBG, THC, CBDV, CBGV, CBL, CBN, CBC, THCV, or combinations thereof.

81. The pharmaceutical composition of claim 76, wherein the composition comprises at least two non-acidic cannabinoid compounds.

82. The pharmaceutical composition of claim 76, wherein the non-acidic cannabinoid compound is added in a purified form.

83. The pharmaceutical composition of claim 76, wherein at least one acidic cannabinoid compound and at least one non-acidic cannabinoid compound is present at a molar ratio of between 1:1 and 1:200.

84. The pharmaceutical composition of claim 76, wherein at least one non-acidic cannabinoid compound and at least one acidic cannabinoid compound is present at a molar ratio of between 1:1 and 1:200.

85. A method of treating or ameliorating pain, the method comprising administering to a patient in need thereof a therapeutically amount of a pharmaceutical composition of any one of claims 76-84, the pharmaceutical composition comprising at least one acidic cannabinoid compound, at least one non-acidic cannabinoid compound and a pharmaceutical excipient.

86. A composition produced by:
a. extracting a first feedstock from a cannabis plant to provide a first feedstock comprising an acidic cannabinoid;
b. extracting a second feedstock from the cannabis plant and decarboxylating the extracted second feedstock to provide a second feedstock comprising a non-acidic cannabinoid;
c. combining at least a fraction of the first feedstock comprising an acidic cannabinoid with at least a fraction of the second feedstock comprising a non-acidic cannabinoid thereby producing a composition comprising at least one acidic cannabinoid and at least one non-acidic cannabinoid.

87. The composition prepared in accordance with claim 86, wherein the acidic cannabinoid is CBGA.

88. The composition prepared in accordance with claim 86, wherein the non-acidic cannabinoid is CBG, CBD, CBDV or combinations thereof.

89. The composition prepared in accordance with claim 86, wherein the first feedstock is extracted using supercritical fluid, solvent, ultrasound-assisted, or combinations thereof.

90. The composition prepared in accordance with claim 86, wherein the first feedstock is extracted using supercritical carbon dioxide.

91. The composition prepared in accordance with claim 86, wherein the second feedstock is extracted using supercritical fluid, solvent, ultrasound-assisted, microwave-assisted or combinations thereof, and heated to at least 100° C. for a predetermined time period.

92. The composition prepared in accordance with claim 86, wherein the composition reduces an influx of extracellular Ca²⁺ in a cell as compared to the individual acidic cannabinoid and non-acidic cannabinoid extracts.

93. The composition prepared in accordance with claim 86, wherein the composition modulates an inflammatory response in the cell by modulating the uptake of cytosolic Ca²⁺.

94. The composition prepared in accordance with claim 86, wherein the composition modulates an inflammatory response in the cell by modulating the extrusion of cytosolic Ca²⁺.

95. The composition prepared in accordance with claim 86, wherein the composition modulates an inflammatory response in an immune cell.

96. The composition prepared in accordance with claim 86, wherein the acidic cannabinoid comprises cannabigerolic acid (CBGA) and the non-acidic cannabinoid comprises cannabidiol (CBD), wherein the molar ratio of CBGA to CBD is from about 1:1 to about 1:200.

97. The composition prepared in accordance with claim 86, wherein the non-acidic cannabinoid comprises cannabidiol acid (CBD) and the acidic cannabinoid comprises cannabigerolic acid (CBGA), wherein the molar ratio of CBD to CBGA is from about 1:1 to about 1:200.

98. The composition prepared in accordance with claim 86, wherein the acidic cannabinoid comprises cannabigerolic acid (CBGA) and the non-acidic cannabinoid comprises cannabigerol (CBG), wherein the molar ratio of CBGA to CBG is from about 1:1 to about 1:200.

99. The composition prepared in accordance with claim 86, wherein the non-acidic cannabinoid comprises cannabidivarin (CBDV) and the acidic cannabinoid comprises cannabigerolic acid (CBGA), wherein the molar ratio of CBDV to CBGA is from about 1:1 to about 1:200.

100. The composition prepared in accordance with claim 86, wherein the acidic cannabinoid comprises cannabigerolic acid (CBGA) and the non-acidic cannabinoid comprises cannabidivarin (CBDV), wherein the molar ratio of CBGA to CBDV is from about 1:1 to about 1:200.

101. The composition prepared in accordance with claim 86, wherein the composition is formulated for administration to a subject.

102. The composition prepared in accordance with claim 101, wherein the composition is packaged into a container selected from the group consisting of a tube, ajar, a vial, a bag, a tray, a drum, a bottle, a syringe, and a can.

103. The composition prepared in accordance with claim 102, wherein the container contains information describing directions for use in a subject.

104. The composition prepared in accordance with claim 103, wherein the subject is a human.
